(19) United States
(12) Patent Application Publication
(10) Pub. No.: US 2015/0087586 A1
(43) Pub. Date: Mar. 26, 2015

(54) PEGYLATED C-PEPTIDE

(71) Applicant: Cebix, Inc., San Diego, CA (US)

(72) Inventors: Sheri Barrack, La Jolla, CA (US); James Callaway, San Diego, CA (US); Michelle Mazzoni, San Diego, CA (US)

(21) Appl. No.: 14/388,502

(22) PCT Filed: Mar. 25, 2013
(86) PCT No.: PCT/US2013/033713

§ 371 Date: Sep. 26, 2014

Related U.S. Application Data
(60) Provisional application No. 61/617,311, filed on Mar. 29, 2012.

(51) Int. Cl. A61K 47/48; C07K 14/765

(52) U.S. Cl. CPC A61K 47/48284; A61K 47/48215; C07K 14/765; A61K 38/00
USPC 514/6.5; 530/363; 514/15.2; 514/6.9

(57) ABSTRACT

The present invention relates to modified forms of C-peptide, and methods for their use. In one aspect, the modified forms of C-peptide comprise conjugated C-peptide derivatives which exhibit superior pharmacokinetic and biological activity in vivo.

### CROSS REFERENCE TO RELATED APPLICATIONS

This application claims the benefit of priority of U.S. provisional application No. 61/617,311, filed Mar. 29, 2012, the disclosure of which is incorporated by reference as if written herein in its entirety.

### BACKGROUND OF THE INVENTION

### Field of the Invention

The present invention relates to modified forms of C-peptide, and methods for their use.

C-peptide is the linking peptide between the A- and B-chains in the proinsulin molecule. After cleavage and processing in the endoplasmic reticulum of pancreatic islet β-cells, insulin and C-peptide are generated. C-peptide is co-secreted with insulin in equimolar amounts from the pancreatic islet β-cells into the portal circulation. Besides its contribution to the folding of the two-chain insulin structure, further biologic activity of C-peptide was questioned for many years after its discovery.

Type 1 diabetes, or insulin-dependent diabetes mellitus, is generally characterized by insulin and C-peptide deficiency, due to an autoimmune destruction of the pancreatic islet β-cells. The patients are therefore dependent on exogenous insulin to sustain life. Several factors may be of importance for the pathogenesis of the disease, e.g., genetic background, environmental factors, and an aggressive autoimmune reaction following a temporary infection (Akerblom H K et al.: Annual Medicine 29(5): 383-385, (1997)). Currently insulin-dependent diabetics are provided with exogenous insulin which has been separated from the C-peptide, and thus do not receive exogenous C-peptide therapy. By contrast most type 2 diabetics initially still produce both insulin and C-peptide endogenously, but are generally characterized by insulin resistance in skeletal muscle and adipose tissue.

Type 1 diabetics suffer from a constellation of long-term complications of diabetes that are in many cases more severe and widespread than in type 2 diabetes. Specifically, for example microvascular complications involving the retina, kidneys, and nerves are a major cause of morbidity and mortality in patients with type 1 diabetes.

There is increasing support for the concept that C-peptide deficiency may play a role in the development of the long-term complications of insulin-dependent diabetics. Additionally, in vivo as well as in vitro studies, in diabetic animal models and in patients with type 1 diabetes, demonstrate that C-peptide possesses hormonal activity (Wahren J et al.: American Journal of Physiology 278: E759-E768, (2000); Wahren J et al.: In International textbook of diabetes mellitus Ferranninni E, Zimmet P, De Fronzo R A, Keen H, Eds. Chichester, John Wiley & Sons, (2004), p. 165-182). Thus, C-peptide used as a complement to regular insulin therapy may provide an effective approach to the management of type 1 diabetes long-term complications.

Studies to date suggest that C-peptide's therapeutic activity involves the binding of C-peptide to a G-protein-coupled membrane receptor, activation of Ca²⁺-dependent intracellular signalling pathways, and phosphorylation of the MAP-kinase system, eliciting increased activities of sodium/potassium ATPase and endothelial nitric oxide synthase (eNOS). Despite the promise of using C-peptide to treat and prevent the long-term complications of insulin-dependent diabetes, the short biological half-life and requirement to dose C-peptide multiple times per day via subcutaneous injection, or intravenous (I.V.) administration, has hindered commercial development.

The present invention is focused on the development of modified versions of C-peptide that retain the biological activity of the native C-peptide and exhibit superior pharmacokinetic properties. These improved therapeutic forms of C-peptide enable the development of more effective therapeutic regimens for the treatment of the long-term complications of diabetes, and require significantly less frequent administration.

In one aspect, these therapies are targeted to diabetic patients, and in a further aspect to insulin-dependent patients. In one aspect, the insulin-dependent patients are suffering from one or more long-term complications of diabetes.

These improved methods are based on modifications of C-peptide that result in versions of C-peptide that retain the biological activity of the native molecule, while exhibiting superior pharmacokinetic characteristics.

### SUMMARY OF THE INVENTION

In one embodiment, the present invention includes a PEGylated C-peptide comprising a PEG moiety covalently attached to the N-terminus of C-peptide. In one aspect, the PEGylated C-peptide of the invention comprises a linear polymer PEG polymer. In another aspect, the PEGylated C-peptide of the invention comprises a branched chain PEG polymer.

In another aspect of any of these PEGylated C-peptides, the PEG moiety has a molecular weight of between about 10 kDa and about 80 kDa. In another aspect, the PEG moiety has a molecular weight of between about 20 kDa and about 60 kDa. In another aspect, the PEG moiety has a molecular weight of between about 30 kDa and about 50 kDa.

In another aspect of any of these PEGylated C-peptides, the PEGylated C-peptide has a structure selected from the group consisting of structural Formula I and structural Formula II:

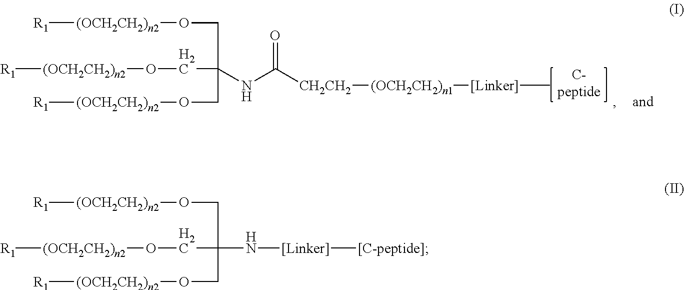

wherein:
R₁=alkyl;
n₁ is 1 to 12;
n₂ is 1 to 800;

the linker is selected from the group consisting of:
—X—CO—(CH₂)ₘ₁, —(CH₂)ₘ₁—CO—X—,
—X—(CH₂)ₘ₁—CO—X—, —X—CO—(CH₂)ₘ₁X—,
—X—CO—(CH₂)ₘ₁—CO—X—(CH₂)ₘ₁—X—CO—, —X—(CH₂)ₘ₁—X—CO—(CH₂)ₘ₂—,
—X—(CH₂)ₘ₁—CO—X—(CH₂)ₘ₂—, —X—(CH₂)ₘ₁—X—CO—(CH₂)ₘ₂—X—,
—X—(CH₂)ₘ₁—X—, —CO—(CH₂)ₘ₁—CO—,
—X—CO—(CH₂)ₘ₁—, —(CH₂)ₘ₁—CO—X—,
—X—(CH₂)ₘ₁—CO—X—, —X—CO—(CH₂)ₘ₁X—,
—X—CO—(CH₂)ₘ₁—CO—X—(CH₂)ₘ₁—X—CO—, —X—(CH₂)ₘ₁—X—CO—(CH₂)ₘ₂—,
—X—(CH₂)ₘ₁—CO—X—(CH₂)ₘ₂—, —X—(CH₂)ₘ₁—X—CO—(CH₂)ₘ₂—X—,
—X—(CH₂)ₘ₁—X—CO—(CH₂)ₘ₂—CO—,
—X—(CH₂)ₘ₁—CO—X—(CH₂)ₘ₂—X—, and
—X—(CH₂)ₘ₁—CO—X—(CH₂)ₘ₂—CO—;

wherein each X is independently selected from —O—, —S—, or NH— or is missing;
each m₁ is independently 0 to 5;
each m₂ is independently 1 to 5; and wherein the linker is attached to the N-terminal amino group of C-peptide.

In another aspect of any of these PEGylated C-peptides, the PEGylated C-peptide has structural Formula III:

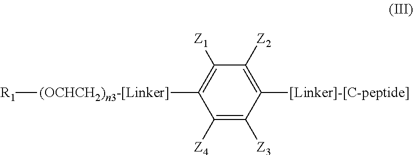

wherein:
R₁=alkyl;
n₁ is 1 to 12;
n₃ is 20 to 800;
Z₁, Z₂, Z₃, and Z₄ are independently selected from the group consisting of hydrogen and alkyl;

the linker is selected from the group consisting of:
—X—, —CO—, —(CH₂)ₘ₂,
—(CH₂)ₘ₁—CO—, —CO—(CH₂)ₘ₁—,
—CO—X—CO—, —(CH₂)ₘ₁—X—(CH₂)ₘ₁—,
—(CH₂)ₘ₁—CO—(CH₂)ₘ₁—, —X—CO—X—,
—X—(CH₂)ₘ₁—X—, —CO—(CH₂)ₘ₁—CO—,
—X—CO—(CH₂)ₘ₁—, —(CH₂)ₘ₁—CO—X—,
—X—(CH₂)ₘ₁—CO—X—, —X—CO—(CH₂)ₘ₁X—,
—X—CO—(CH₂)ₘ₁—CO—X—(CH₂)ₘ₁—X—CO—, —X—(CH₂)ₘ₁—X—CO—(CH₂)ₘ₂—,
—X—(CH₂)ₘ₁—CO—X—(CH₂)ₘ₂—, —X—(CH₂)ₘ₁—X—CO—(CH₂)ₘ₂—X—,
—X—(CH₂)ₘ₁—X—CO—(CH₂)ₘ₂—CO—,
—X—(CH₂)ₘ₁—CO—X—(CH₂)ₘ₂—X—, and
—X—(CH₂)ₘ₁—CO—X—(CH₂)ₘ₂—CO—;

wherein each X is independently selected from —O—, —S—, or NH— or is missing;
each m₁ is independently 0 to 5;
each m₂ is independently 1 to 5; and wherein the linker is attached to the N-terminal amino group of C-peptide.

In another aspect of any of these PEGylated C-peptides, the PEGylated C-peptide has structural Formula IV:

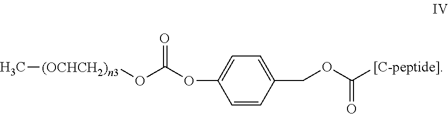

In another aspect of any of these PEGylated C-peptides, the PEGylated C-peptide has structural Formula V:

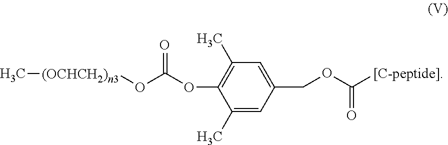

In another aspect of any of these PEGylated C-peptides, the PEGylated C-peptide has structural Formula VI:

CH₃(CH₂)₇CH═CH(CH₂)₈—O—(CH₂CH₂O)ₙ₁-[Linker]-[C-peptide] (VI)

wherein: n₁ is 20 to 800;

the linker is selected from the group consisting of:
—X—, —CO—, —(CH₂)ₘ₂—,
—(CH₂)ₘ₁—CO—, —CO—(CH₂)ₘ₁—,
—CO—X—CO—, —(CH₂)ₘ₁—X—(CH₂)ₘ₁—,
—(CH₂)ₘ₁—CO—(CH₂)ₘ₁—, —X—CO—X—,
—X—(CH₂)ₘ₁—X—, —CO—(CH₂)ₘ₁—CO—,
—X—CO—(CH₂)ₘ₁—, —(CH₂)ₘ₁—CO—X—,
—X—(CH₂)ₘ₁—CO—X—, —X—CO—(CH₂)ₘ₁X—,
—X—(CH₂)ₘ₁—CO—X—(CH₂)ₘ₂—,
—X—(CH₂)ₘ₁—X—CO—(CH₂)ₘ₂—CO—,
—X—(CH₂)ₘ₁—CO—X—(CH₂)ₘ₂—X—, and
—X—(CH₂)ₘ₁—CO—X—(CH₂)ₘ₂—CO—;

wherein each X is independently selected from —O—, —S—, or —NH— or is missing;
each m₁ is independently 0 to 5;
each m₂ is independently 1 to 5; and wherein the linker is attached to the N-terminal amino group of C-peptide.

In another aspect of any of these PEGylated C-peptides, the PEGylated C-peptide has structural Formula VII:

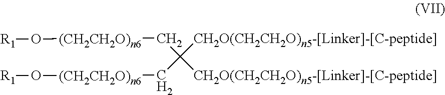

wherein:
R₁=alkyl;
n₅ is 1 to 12;
n₆ is 20 to 800;

the linker is selected from the group consisting of:
—X—, —CO—, —(CH₂)ₘ₂—,
—(CH₂)ₘ₁—CO—, —CO—(CH₂)ₘ₁—,
—CO—X—CO—, —(CH₂)ₘ₁—X—(CH₂)ₘ₁—,
—(CH₂)ₘ₁—CO—(CH₂)ₘ₁—, —X—CO—X—,
—X—(CH₂)ₘ₁—X—, —CO—(CH₂)ₘ₁—CO—,
—X—CO—(CH₂)ₘ₁—, —(CH₂)ₘ₁—CO—X—,
—X—(CH₂)ₘ₁—CO—X—, —X—CO—(CH₂)ₘ₁X—,
—X—CO—(CH₂)ₘ₁—CO—X—(CH₂)ₘ₁—X—CO—, —X—(CH₂)ₘ₁—X—CO—(CH₂)ₘ₂—,
—X—(CH₂)ₘ₁—CO—X—(CH₂)ₘ₂—, —X—(CH₂)ₘ₁—X—CO—(CH₂)ₘ₂—X—,
—X—(CH₂)ₘ₁—X—CO—(CH₂)ₘ₂—CO—,
—X—(CH₂)ₘ₁—CO—X—(CH₂)ₘ₂—X—, and
—X—(CH₂)ₘ₁—CO—X—(CH₂)ₘ₂—CO—;

wherein each X is independently selected from —O—, —S—, or NH— or is missing;
each m₁ is independently 0 to 5;
each m₂ is independently 1 to 5; and wherein the linker is attached to the N-terminal amino group of C-peptide.

In another aspect of any of these PEGylated C-peptides, the PEGylated C-peptide has structural Formula VIII:

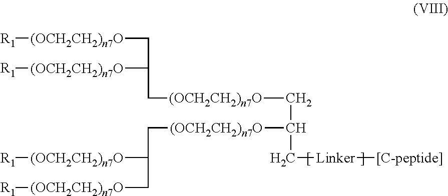

wherein:
R₁=alkyl;
n₇ is 1 to 800;

the linker is selected from the group consisting of:
—X—, —CO—, —(CH₂)ₘ₂—,
—(CH₂)ₘ₁—CO—, —CO—(CH₂)ₘ₁—,
—CO—X—CO—, —(CH₂)ₘ₁—X—(CH₂)ₘ₁—,
—(CH₂)ₘ₁—CO—(CH₂)ₘ₁—, —X—CO—X—,
—X—(CH₂)ₘ₁—X—, —CO—(CH₂)ₘ₁—CO—,
—X—CO—(CH₂)ₘ₁—, —(CH₂)ₘ₁—CO—X —,
—X—(CH₂)ₘ₁—CO—X—, —X—CO—(CH₂)ₘ₁X—,
—X—CO—(CH₂)ₘ₁—CO—X—(CH₂)ₘ₁—X—CO—, —X—(CH₂)ₘ₁—X—CO—(CH₂)ₘ₂—,
—X—(CH₂)ₘ₁—CO—X—(CH₂)ₘ₂—, —X—(CH₂)ₘ₁—X—CO—(CH₂)ₘ₂—X—,
—X—(CH₂)ₘ₁—X—CO—(CH₂)ₘ₂—CO—,
—X—(CH₂)ₘ₁—CO—X—(CH₂)ₘ₂—X—, and
—X—(CH₂)ₘ₁—CO—X—(CH₂)ₘ₂—CO—;

wherein each X is independently selected from —O—, —S—, or NH— or is missing;
each m₁ is independently 0 to 5;
each m₂ is independently 1 to 5; and wherein the linker is attached to the N-terminal amino group of C-peptide.

In another aspect of any of these PEGylated C-peptides, the PEGylated C-peptide has structural Formula IX:

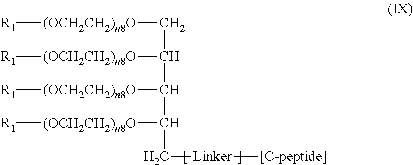

wherein:
R₁=alkyl;
n₇ is 1 to 800;

the linker is selected from the group consisting of:
—X—, —CO—, —(CH₂)ₘ₂—,
—(CH₂)ₘ₁—CO—, —CO—(CH₂)ₘ₁—,
—CO—X—CO—, —(CH₂)ₘ₁—X—(CH₂)ₘ₁—,
—(CH₂)ₘ₁—CO—(CH₂)ₘ₁—, —X—CO—X—,
—X—(CH₂)ₘ₁—X—, —CO—(CH₂)ₘ₁—CO—,
—X—CO—(CH₂)ₘ₁—, —(CH₂)ₘ₁—CO—X—,
—X—(CH₂)ₘ₁—CO—X—, —X—CO—(CH₂)ₘ₁X—,
—X—CO—(CH₂)ₘ₁—CO—X—(CH₂)ₘ₁—X—CO—, —X—(CH₂)ₘ₁—X—CO—(CH₂)ₘ₂—,
—X—(CH₂)ₘ₁—CO—X—(CH₂)ₘ₂—, —X—(CH₂)ₘ₁—X—CO—(CH₂)ₘ₂—X—,
—X—(CH₂)ₘ₁—X—CO—(CH₂)ₘ₂—CO—,
—X—(CH₂)ₘ₁—CO—X—(CH₂)ₘ₂—X—, and
—X—(CH₂)ₘ₁—CO—X—(CH₂)ₘ₂—CO—;

wherein each X is independently selected from —O—, —S—, or NH— or is missing;
each m₁ is independently 0 to 5;
each m₂ is independently 1 to 5; and wherein the linker is attached to the N-terminal amino group of C-peptide.

In another aspect of any of these PEGylated C-peptides, the PEGylated C-peptide has a structure selected from the group consisting of structural Formula X and structural Formula XI:

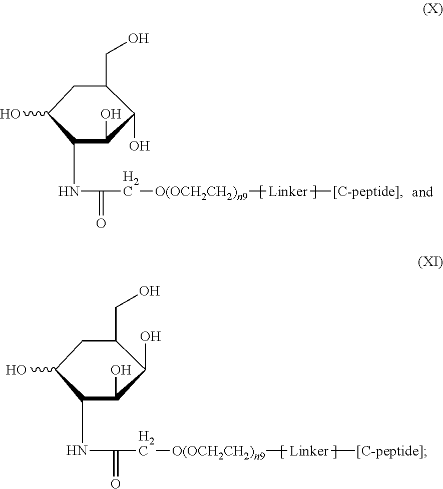

wherein: n₉ is 20 to 800;

the linker is selected from the group consisting of:
—X—, —CO—, —(CH₂)ₘ₂—,
—(CH₂)ₘ₁—CO—, —CO—(CH₂)ₘ₁—,
—CO—X—CO—, —(CH₂)ₘ₁—X—(CH₂)ₘ₁—,
—(CH₂)ₘ₁—CO—(CH₂)ₘ₁—, —X—CO—X—,
—X—(CH₂)ₘ₁—X—, —CO—(CH₂)ₘ₁—CO—,
—X—CO—(CH₂)ₘ₁—, —(CH₂)ₘ₁—CO—X—,
—X—(CH₂)ₘ₁—CO—X—, —X—CO—(CH₂)ₘ₁X—,
—X—CO—(CH₂)ₘ₁—CO—X—(CH₂)ₘ₁—X—CO—, —X—(CH₂)ₘ₁—X—CO—(CH₂)ₘ₂—,
—X—(CH₂)ₘ₁—CO—X—(CH₂)ₘ₂—, —X—(CH₂)ₘ₁—X—CO—(CH₂)ₘ₂—X—,
—X—(CH₂)ₘ₁—X—CO—(CH₂)ₘ₂—CO—,
—X—(CH₂)ₘ₁—CO—X—(CH₂)ₘ₂—X—, and
—X—(CH₂)ₘ₁—CO—X—(CH₂)ₘ₂—CO—;

wherein each X is independently selected from —O—, —S—, or NH— or is missing;
each m₁ is independently 0 to 5;
each m₂ is independently 1 to 5; and wherein the linker is attached to the N-terminal amino group of C-peptide.

In another aspect of any of these PEGylated C-peptides, the PEGylated C-peptide has structural Formula XII:

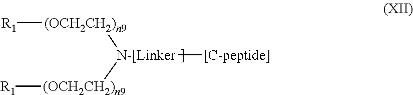

wherein:
R₁=alkyl;
n₉ is 20 to 800;

the linker is selected from the group consisting of:
—X—, —CO—, —(CH₂)ₘ₂—,
—(CH₂)ₘ₁—CO—, —CO—(CH₂)ₘ₁—,
—CO—X—CO—, —(CH₂)ₘ₁—X—(CH₂)ₘ₁—,
—(CH₂)ₘ₁—CO—(CH₂)ₘ₁—, —X—CO—X—,
—X—(CH₂)ₘ₁—X—, —CO—(CH₂)ₘ₁—CO—,
—X—CO—(CH₂)ₘ₁—, —(CH₂)ₘ₁—CO—X—,
—X—(CH₂)ₘ₁—CO—X—, —X—CO—(CH₂)ₘ₁X—,
—X—CO—(CH₂)ₘ₁—CO—X—(CH₂)ₘ₁—X—CO—, —X—(CH₂)ₘ₁—X—CO—(CH₂)ₘ₂—,
—X—(CH₂)ₘ₁—CO—X—(CH₂)ₘ₂—, —X—(OH₂)ₘ₁—X—CO—(CH₂)ₘ₂—X—,
—X—(CH₂)ₘ₁—X—CO—(CH₂)ₘ₂—CO—,
—X—(CH₂)ₘ₁—CO—X—(CH₂)ₘ₂—X—, and
—X—(CH₂)ₘ₁—CO—X—(CH₂)ₘ₂—CO—;

wherein each X is independently selected from —O—, —S—, or NH— or is missing;
each m₁ is independently 0 to 5;
each m₂ is independently 1 to 5; and wherein the linker is attached to the N-terminal amino group of C-peptide.

In another aspect of any of the modified C-peptides, the modified C-peptide has the structural Formula XIII:

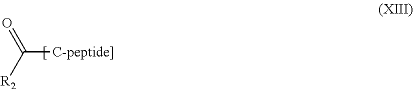

wherein:
The C-peptide is modified at the N-terminus; and
R₂ is selected from the group consisting of alkyl, haloalkyl, perhaloalkyl, heteroalkyl, hydroxyalkyl, alkoxyalkyl, aminoalkyl, alkylaminoalkyl, thioalkyl, arylalkyl, cycloalkylalkyl, heteroarylalkyl, heterocycloalkylalkyl, alkenyl, arylalkenyl, heteroarylalkenyl, heterocycloalkylalkenyl, alkynyl, arylalkynyl, heteroarylalkynyl, heterocycloalkylalkynyl, alkoxy, haloalkoxy, perhaloalkoxy, arylalkoxy, aryloxy, heteroaryloxy, alkylamino, alkylthio, arylthio, heteroarylthio, aryl, heteroaryl, cycloalkyl, and heterocycloalkyl, any of which may be optionally substituted with a substituent selected from the group consisting of hydrogen, halogen, hydroxy, cyano, nitro, alkyl, haloalkyl, perhaloalkyl, heteroalkyl, hydroxyalkyl, alkoxyalkyl, aminoalkyl, alkylaminoalkyl, thioalkyl, arylalkyl, cycloalkylalkyl, heteroarylalkyl, heterocycloalkylalkyl, alkenyl, arylalkenyl heteroarylalkenyl, heterocycloalkylalkenyl, alkynyl, arylalkynyl heteroarylalkynyl, heterocycloalkylalkynyl, alkoxy, haloalkoxy, perhaloalkoxy, acyloxy, arylalkoxy, aryloxy, heteroaryloxy, acyl, arylalkanoyl, alkylcarbonyl, alkoxycarbonyl, carboxyl, amino, alkylamino, arylamino, C-amido, N-amido, carbamate, urea, N-sulfonamido, S-sulfonamido, alkylsulfonyl, thiol, alkylthio, arylthio, heteroarylthio, aryl, heteroaryl, cycloalkyl, and heterocycloalkyl.

In another aspect of any of the modified C-peptides, R₂ is alkyl.

In another aspect of any of the modified C-peptides, R₂ is unsubstituted C₈-C₂₀ alkyl.

In another aspect of any of the modified C-peptides, R₂ is selected from the group consisting of cis-CH₃(CH₂)₃CH═CH(CH₂)₇—, cis-CH₃(CH₂)₅CH═CH(CH₂)₇—, cis-CH₃(CH₂)₇CH═CH(CH₂)₇—, cis,cis-CH₃(CH₂)₄CH═CHCH₂CH═CH(CH₂)₇—, CH₃(CH₂)₆—, CH₃(CH₂)₈—, CH₃(CH₂)₁₀—, CH₃(CH₂)₁₂—, CH₃(CH₂)₁₄—, CH₃(CH₂)₁₆—, CH₃(CH₂)₁₈—, and CH₃(CH₂)₂₀—.

In another aspect of any of the modified C-peptides, the modified C-peptide has the structural Formula XIV:

[Human Serum Albumin]-[Linke]-[C-peptide] (XIV)

wherein:
the linker is selected from the group consisting of:
-Q₁-(CH₂)ₘ₁—CO—,
—CO—X—CO—, -Q₁-(CH₂)ₘ₁—X—(CH₂)ₘ₁—,
-Q₁-(CH₂)ₘ₁—CO—(CH₂)ₘ₁—, -Q₁-X—CO—X—,
-Q₁-X—(CH₂)ₘ₁—X—, —CO—(CH₂)ₘ₁—CO—,
-Q₁-X—CO—(CH₂)ₘ₁—, -Q₁-(CH₂)ₘ₁—CO—X—,
-Q₁-X—(CH₂)ₘ₁—CO—X—, -Q₁-X—CO—(CH₂)ₘ₁X—,
-Q₁-X—CO—(CH₂)ₘ₁—CO—X—(CH₂)ₘ₁—X—CO—, -Q₁-X—(CH₂)ₘ₁—X—CO—(CH₂)ₘ₂—,
-Q₁-X—(CH₂)ₘ₁—CO—X—(CH₂)ₘ₂—, -Q₁-X—(CH₂)ₘ₁—X—CO—(CH₂)ₘ₂—X—,
-Q₁-X—(CH₂)ₘ₁—X—CO—(CH₂)ₘ₂—CO—,
-Q₁-X—(CH₂)ₘ₁—CO—X—(CH₂)ₘ₂—X—, and
-Q₁-X—(CH₂)ₘ₁—CO—X—(CH₂)ₘ₂—CO—;

wherein Q₁ is absent or selected from the group consisting of —CO— or

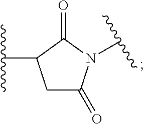 [SMILES: *C1CC(=O)N(*)C1=O]

each X is independently selected from —O—, —S—, or NH— or is missing;
each m₁ is independently 0 to 5;
each m₂ is independently 1 to 5; and
wherein the linker is attached to the N-terminal amino group of C-peptide.

In another aspect of any of the modified C-peptides, the modified C-peptide the human serum albumin is modified at the 34-cysteine.

In another aspect of any of the modified C-peptides, the modified C-peptide has the structure:

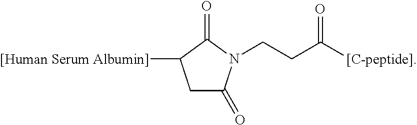

In another aspect of any of the modified C-peptides, the modified C-peptide has the structure:

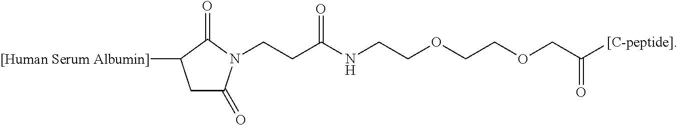

In another aspect of any of the modified C-peptides, the modified C-peptide has the structure:

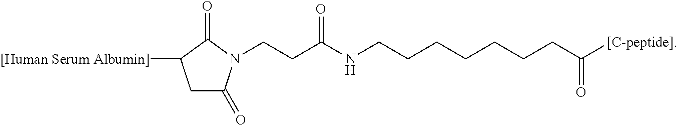

In another aspect of any of the modified C-peptides, the modified C-peptide has the structure:

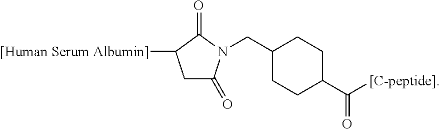

In another aspect of any of the modified C-peptides, the modified C-peptide has the structural Formula XV:

[Hydroxyethyl Starch]-[Linker]-[C-peptide] (XV)

the linker is selected from the group consisting of:
—X—, —CO—, —(CH₂)ₘ₂—,
—(CH₂)ₘ₁—CO—, —CO—(CH₂)ₘ₁—,
—CO—X—CO—, —(CH₂)ₘ₁—X—(CH₂)ₘ₁—,
—(CH₂)ₘ₁—CO—(CH₂)ₘ₁—, —X—CO—X—,
—X—(CH₂)ₘ₁—X—, —CO—(CH₂)ₘ₁—CO—,
—X—CO—(CH₂)ₘ₁—, —(CH₂)ₘ₁—CO—X—,
—X—(CH₂)ₘ₁—CO—X—, —X—CO—(CH₂)ₘ₁ X—,
—X—CO—(CH₂)ₘ₁—CO—X—(CH₂)ₘ₁—X—CO—, —X—(CH₂)ₘ₁—X—CO—(CH₂)ₘ₂—,
—X—(CH₂)ₘ₁—CO—X—(CH₂)ₘ₂—, —X—(CH₂)ₘ₁—X—CO—(CH₂)ₘ₂—X—,
—X—(CH₂)ₘ₁—X—CO—(CH₂)ₘ₂—CO—,
—X—(CH₂)ₘ₁—CO—X—(CH₂)ₘ₂—X—, and
—X—(CH₂)ₘ₁—CO—X—(CH₂)ₘ₂—CO—;

wherein each X is independently selected from —O—, —S—, or NH— or is missing;
each m₁ is independently 0 to 5;
each m₂ is independently 1 to 5; and wherein the linker is attached to the N-terminal amino group of C-peptide.

In another aspect, the modified C-peptide has the structure:

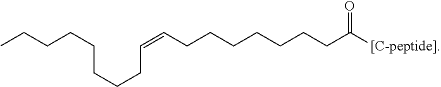

In another aspect of any of the modified C-peptides, the molecular weight of the hydroxyethyl starch is about 70 kDa.

In another aspect of any of the modified C-peptides, the molecular weight of the hydroxyethyl starch is about 70 kDa to 200 kDa.

In another aspect of any of the modified C-peptides, the molecular weight of the hydroxyethyl starch is about 130 kDa.

In another aspect of any of the modified C-peptides, the molecular weight of the hydroxyethyl starch is about 200 kDa.

In another aspect, a composition comprises hydroxyethyl starch and the modified C-peptide of formula XV, wherein the modified C-peptide comprises a molar percentage of about 0.3% to about 0.5%.

In another aspect of any of the modified C-peptides, the C-peptide comprises the pentapeptide sequence (EGSLQ) (SEQ. ID. No. 31).

In certain aspects of any of the claimed conjugated C-peptides, the Conjugated C-peptide has a plasma or sera pharmacokinetic AUC profile at least about 5-fold greater than unmodified C-peptide when subcutaneously administered to dogs.

In certain aspects of any of the claimed conjugated C-peptides, the conjugated C-peptide has a plasma or sera pharmacokinetic AUC profile at least about 6-fold greater than unmodified C-peptide when subcutaneously administered to dogs.

In certain aspects of any of the claimed conjugated C-peptides, the conjugated C-peptide has a plasma or sera pharmacokinetic AUC profile at least about 7-fold greater than unmodified C-peptide when subcutaneously administered to dogs.

In certain aspects of any of the claimed conjugated C-peptides, the conjugated C-peptide has a plasma or sera pharmacokinetic AUC profile at least about 8-fold greater than unmodified C-peptide when subcutaneously administered to dogs.

In certain aspects of any of the claimed conjugated C-peptides, the conjugated C-peptide has a plasma or sera pharmacokinetic AUC profile at least about 10-fold greater than unmodified C-peptide when subcutaneously administered to dogs.

In certain aspects of any of the claimed conjugated C-peptides, the conjugated C-peptide has a plasma or sera pharmacokinetic AUC profile at least about 15-fold greater than unmodified C-peptide when subcutaneously administered to dogs.

In certain aspects of any of the claimed conjugated C-peptides, the conjugated C-peptide has a plasma or sera pharmacokinetic AUC profile at least about 20-fold greater than unmodified C-peptide when subcutaneously administered to dogs.

In certain aspects of any of the claimed conjugated C-peptides, the conjugated C-peptide has a plasma or sera pharmacokinetic AUC profile at least about 25-fold greater than unmodified C-peptide when subcutaneously administered to dogs.

In certain aspects of any of the claimed conjugated C-peptides, the conjugated C-peptide has a plasma or sera pharmacokinetic AUC profile at least about 50-fold greater than unmodified C-peptide when subcutaneously administered to dogs.

In certain aspects of any of the claimed conjugated C-peptides, the conjugated C-peptide has a plasma or sera pharmacokinetic AUC profile at least about 75-fold greater than unmodified C-peptide when subcutaneously administered to dogs.

In certain aspects of any of the claimed conjugated C-peptides, the conjugated C-peptide has a plasma or sera pharmacokinetic AUC profile at least about 100-fold greater than unmodified C-peptide when subcutaneously administered to dogs.

In certain aspects of any of the claimed conjugated C-peptides, the conjugated C-peptide has an equi-potent biological activity with the unmodified C-peptide. In certain aspects of any of the claimed conjugated C-peptides, the conjugated C-peptide retains at least about 95% of the biological activity of the unmodified C-peptide. In certain aspects of any of the claimed conjugated C-peptides, the conjugated C-peptide retains at least about 90% of the biological activity of the unmodified C-peptide. In certain aspects of any of the claimed conjugated C-peptides, the conjugated C-peptide retains at least about 80% of the biological activity of the unmodified C-peptide. In another aspect of any of the claimed conjugated C-peptides, the conjugated C-peptide retains at least about 70% of the biological activity of the unmodified C-peptide. In another aspect of any of the claimed conjugated C-peptides, the conjugated C-peptide retains at least about 60% of the biological activity of the unmodified C-peptide. In another aspect of any of the claimed conjugated C-peptides, the conjugated C-peptide retains at least about 50% of the biological activity of the unmodified C-peptide. In another aspect of any of the claimed conjugated C-peptides, the conjugated C-peptide retains at least about 40% of the biological activity of the unmodified C-peptide. In another aspect of any of the claimed conjugated C-peptides, the conjugated C-peptide retains at least about 30% of the biological activity of the unmodified C-peptide. In another aspect of any of the claimed conjugated C-peptides, the conjugated C-peptide retains at least about 20% of the biological activity of the unmodified C-peptide. In another aspect of any of the claimed conjugated C-peptides, the conjugated C-peptide retains at least about 10% of the biological activity of the unmodified C-peptide. In another aspect of any of the claimed conjugated C-peptides, the conjugated C-peptide retains at least about 5% of the biological activity of the unmodified C-peptide.

In another embodiment, the present invention includes a dosing regimen which maintains an average steady-state concentration of conjugated C-peptide in the patient's plasma of between about 0.2 nM and about 6 nM when using a dosing interval of 3 days or longer, comprising administering to the patient a therapeutic dose of conjugated C-peptide of any of the claimed conjugated C-peptides.

In another embodiment, the present invention includes a dosing regimen which maintains an average steady-state concentration of conjugated C-peptide in the patient's plasma of between about 0.4 nM and about 6 nM when using a dosing interval of 3 days or longer, comprising administering to the patient a therapeutic dose of conjugated C-peptide of any of the claimed conjugated C-peptides.

In another embodiment, the present invention includes a dosing regimen which maintains an average steady-state concentration of conjugated C-peptide in the patient's plasma of between about 0.6 nM and about 8 nM when using a dosing interval of 3 days or longer, comprising administering to the patient a therapeutic dose of conjugated C-peptide of any of the claimed conjugated C-peptides.

In another embodiment, the present invention includes a dosing regimen which maintains an average steady-state concentration of conjugated C-peptide in the patient's plasma of between about 0.8 nM and about 10 nM when using a dosing interval of 3 days or longer, comprising administering to the patient a therapeutic dose of conjugated C-peptide of any of the claimed conjugated C-peptides.

In another embodiment, the present invention includes a method for maintaining C-peptide levels above the minimum effective therapeutic level in a patient in need thereof, comprising administering to the patient a therapeutic dose of any of the claimed conjugated C-peptides.

In another aspect of any of the claimed conjugated C-peptides, the conjugated C-peptide is substantially free of adverse side effects when subcutaneously administered to a mammal at an effective therapeutic dose.

In another embodiment, the present invention includes a method for treating one or more long-term complications of diabetes in a patient in need thereof, comprising administering to the patient a therapeutic dose of any of the claimed conjugated C-peptides.

In another embodiment, the present invention includes a method for treating a patient with diabetes comprising administering to the patient a therapeutic dose of conjugated C-peptide of any of the claimed conjugated C-peptides in combination with insulin.

In one aspect of any of these methods, the conjugated C-peptide is administered with a dosing interval of about 3 days or longer. In one aspect of any of these methods, the conjugated C-peptide is administered with a dosing interval of about 4 days or longer. In one aspect of any of these methods, the conjugated C-peptide is administered with a dosing interval of about 5 days or longer. In one aspect of any of these methods, the conjugated C-peptide is administered with a dosing interval of about 6 days or longer. In one aspect of any of these methods, the conjugated C-peptide is administered with a dosing interval of about 7 days or longer.

In certain embodiments, treatment results in an improvement of at least 10% in nerve conduction velocity compared to nerve conduction velocity prior to starting conjugated C-peptide therapy.

In another aspect of any of these methods, the plasma concentration of conjugated C-peptide is maintained above about 0.1 nM. In another aspect of any of these methods, the plasma concentration of conjugated C-peptide is maintained above about 0.2 nM. In another aspect of any of these methods, the plasma concentration of conjugated C-peptide is maintained above about 0.3 nM. In another aspect of any of these methods, the plasma concentration of conjugated C-peptide is maintained above about 0.4 nM.

In another aspect of any of these methods, the therapeutic dose of conjugated C-peptide is administered subcutaneously. In another aspect of any of these methods, the therapeutic dose of conjugated C-peptide is administered orally.

In another embodiment, the present invention includes the use of any of the claimed conjugated C-peptides as a C-peptide replacement therapy in a patient in need thereof.

In another embodiment, the present invention includes the use of any of the claimed conjugated C-peptides for treating one or more long-term complications of diabetes in a patient in need thereof. In certain embodiments, the long-term complications of diabetes are selected from the group consisting of retinopathy, peripheral neuropathy, autonomic neuropathy, nephropathy and erectile dysfunction. In certain embodiments, the long-term complication of diabetes is peripheral neuropathy. In certain embodiments, the peripheral neuropathy is established peripheral neuropathy. In certain embodiments, treatment results in an improvement of at least 10% in nerve conduction velocity compared to nerve conduction velocity prior to starting conjugated C-peptide therapy.

In another embodiment, the present invention includes a pharmaceutical composition comprising any of the claimed conjugated C-peptides and a pharmaceutically acceptable carrier or excipient. In certain embodiments, the pharmaceutically acceptable carrier or excipient is sorbitol. In certain embodiments, the sorbitol is present at a concentration of about 2% to about 8% wt/wt. In certain embodiments, the sorbitol is present at a concentration of about 4.7%. In certain embodiments, the pharmaceutical composition is buffered to a pH within the range of about pH 5.5 to about pH 6.5. In certain embodiments, the pharmaceutical composition is buffered to a pH of about 6.0. In certain embodiments, the pharmaceutical composition is buffered with a phosphate buffer at a concentration of about 5 mM to about 25 mM. In certain embodiments, the pharmaceutical composition is buffered with a phosphate buffer at a concentration of about 10 mM. In one aspect of any of these embodiments, the pharmaceutical composition is characterized by improved stability of any of the claimed conjugated C-peptides compared to a pharmaceutical composition comprising the same conjugated C-peptide and 0.9% saline at pH 7.0, wherein the stability is determined after incubation for a predetermined time at 40° C. In different embodiments, the pre-determined time is about one week, about 2 weeks, about three weeks, about four weeks, or about five weeks, or about six weeks.

In another embodiment, the present invention includes a pharmaceutical composition comprising any of the claimed conjugated C-peptides and insulin.

Certain embodiments include the use of any of the disclosed conjugated C-peptides to reduce the risk of hypoglycemia in a human patient with insulin dependent diabetes, in a regimen which additionally comprises the administration of insulin, comprising; a) administering insulin to the patient; b) administering a therapeutic dose of the conjugated C-peptide in a different site as that used for the patient's insulin administration; c) adjusting the dosage amount, type, or frequency of insulin administered based on the patient's altered insulin requirements resulting from the therapeutic dose of the conjugated C-peptide.

In some embodiments, the patient has at least one long term complications of diabetes.

Certain embodiments include a method for treating an insulin-dependent human patient, comprising the steps of; a) administering insulin to the patient, wherein the patient has neuropathy; b) administering subcutaneously to the patient a therapeutic dose of any of the disclosed conjugated C-peptides in a different site as that used for the patient's insulin administration; c) adjusting the dosage amount, type, or frequency of insulin administered based on monitoring the patient's altered insulin requirements resulting from the therapeutic dose of conjugated C-peptide, wherein the adjusted dose of insulin reduces the risk, incidence, or severity of hypoglycemia, wherein the adjusted dose of insulin is at least 10% less than the patient's insulin dose prior to starting conjugated C-peptide treatment.

Certain embodiments include a method of reducing insulin usage in an insulin-dependent human patient, comprising the steps of; a) administering insulin to the patient; b) administering subcutaneously to the patient a therapeutic dose any of the disclosed conjugated C-peptides in a different site as that used for the patient's insulin administration; c) adjusting the dosage amount, type, or frequency of insulin administered based on monitoring the patient's altered insulin requirements resulting from the therapeutic dose of conjugated C-peptide, wherein the adjusted dose of insulin does not induce hypoglycemia, wherein the adjusted dose of insulin is at least 10% less than the patient's insulin dose prior to starting the conjugated C-peptide treatment.

### DETAILED DESCRIPTION OF THE INVENTION

### Definitions

The term “active” or “activated” when used in conjunction with a particular functional group refers to a reactive functional group that reacts readily with an electrophile or a nucleophile on another molecule. This is in contrast to those groups that require strong catalysts or highly impractical reaction conditions in order to react (i.e., a “non-reactive” or “inert” group). As used herein, the term “functional group” or any synonym thereof is meant to encompass protected forms thereof as well as unprotected forms.

The term “alkoxy” refers to an —O—R group, wherein R is alkyl or substituted alkyl, preferably C1-6 alkoxy (e.g., methoxy, ethoxy, propyloxy, and so forth).

The term “alkyl” refers to a hydrocarbon, typically ranging from about 1 to 12 atoms in length. Hydrocarbons may be branched or linear and are preferably, but not necessarily saturated. Exemplary alkyl groups include methyl, ethyl, propyl, butyl, pentyl, 2-methylbutyl, 2-ethylpropyl, etc. As used herein “alkyl” includes cycloalkyl as well as cycloalkylene alkyls. The term “lower alkyl” refers to an alkyl group containing from 1 to 6 carbon atoms, and may be straight chain or branched.

The term “Cₘₐₓ” as used herein is the maximum serum or plasma concentration of drug which occurs during the period of release which is monitored.

The term “Cₘᵢₙ” as used herein is the minimum serum or plasma concentration of drug which occurs during the period of release during the treatment period.

The term “Cₐᵥₑ” as used herein is the average serum or plasma concentration of drug derived by dividing the area under the curve (AUC) of the release profile by the duration of the release.

The term “Cₛₛ₋ₐᵥₑ” as used herein is the average steady-state concentration of drug obtained during a multiple dosing regimen after dosing for at least five elimination half-lives. It will be appreciated that drug concentrations are fluctuating within dosing intervals even once an average steady-state concentration of drug has been obtained.

The term “tₘₐₓ” as used herein is the time post-dose at which Cₘₐₓ is observed.

The term “AUC” as used herein means “area under curve” for the serum or plasma concentration-time curve, as calculated by the trapezoidal rule over the complete sample collection interval.

The term “bioavailability” refers to the amount of drug that reaches the circulation system expressed in percent of that administered. The amount of bioavailable material can be defined as the calculated AUC for the release profile of the drug during the time period starting at post-administration and ending at a predetermined time point. As is understood in the art, a release profile is generated by graphing the serum levels of a biologically active agent in a subject (Y-axis) at predetermined time points (X-axis). Bioavailability is often referred to in terms of % bioavailability, which is the bioavailability achieved for a drug (such as C-peptide) following administration of a sustained release composition of that drug divided by the bioavailability achieved for the drug following intravenous administration of the same equivalent dose of the drug, multiplied by 100.

The phrase “conservative amino acid substitution” or “conservative mutation” refers to the replacement of one amino acid by another amino acid with a common property. A functional way to define common properties between individual amino acids is to analyze the normalized frequencies of amino acid changes between corresponding proteins of homologous organisms (Schulz G E and R H Schirmer, Principles of Protein Structure, Springer-Verlag (1979)). According to such analyses, groups of amino acids can be defined where amino acids within a group exchange preferentially with each other, and therefore resemble each other most in their impact on the overall protein structure (Schulz G E and R H Schirmer, Principles of Protein Structure, Springer-Verlag (1979)).

Examples of amino acid groups defined in this manner include: a “charged/polar group,” consisting of Glu, Asp, Asn, Gln, Lys, Arg, and His; an “aromatic or cyclic group,” consisting of Pro, Phe, Tyr, and Trp; and an “aliphatic group,” consisting of Gly, Ala, Val, Leu, Ile, Met, Ser, Thr, and Cys.

Within each group, subgroups can also be identified, e.g., the group of charged/polar amino acids can be sub-divided into the subgroups consisting of the “positively-charged subgroup,” consisting of Lys, Arg, and His; the “negatively-charged subgroup,” consisting of Glu and Asp, and the “polar subgroup” consisting of Asn and Gln. The aromatic or cyclic group can be sub-divided into the subgroups consisting of the “nitrogen ring subgroup,” consisting of Pro, His, and Trp; and the “phenyl subgroup” consisting of Phe and Tyr. The aliphatic group can be sub-divided into the subgroups consisting of the “large aliphatic non-polar subgroup,” consisting of Val, Leu, and Ile; the “aliphatic slightly-polar subgroup,” consisting of Met, Ser, Thr, and Cys; and the “small-residue sub-group,” consisting of Gly and Ala.

Examples of conservative mutations include amino acid substitutions of amino acids within the subgroups above, e.g., Lys for Arg and vice versa such that a positive charge can be maintained; Glu for Asp and vice versa such that a negative charge can be maintained; Ser for Thr such that a free —OH can be maintained; and Gln for Asn such that a free —NH₂ can be maintained. “Semi-conservative mutations” include amino acid substitutions of amino acids with the same groups listed above, which do not share the same subgroup. For example, the mutation of Asp for Asn, or Asn for Lys, all involve amino acids within the same group, but different subgroups. “Non-conservative mutations” involve amino acid substitutions between different groups, e.g., Lys for Leu, Phe for Ser.

The terms “Dalton”, “Da”, or “D” refers to an arbitrary unit of mass, being 1/12 the mass of the nuclide of carbon-12, equivalent to 1.657×10⁻²⁴ g. The term “kDa” is for kilodalton (i.e., 1000 Daltons).

The terms “diabetes”, “diabetes mellitus”, or “diabetic condition”, unless specifically designated otherwise, encompass all forms of diabetes. The term “type 1 diabetic” or “type 1 diabetes” refers to a patient with a fasting plasma glucose concentration of greater than about 7.0 mmoUL and a fasting C-peptide level of about, or less than about 0.2 nmoL/L. The term “type 1.5 diabetic” or “type 1.5 diabetes” refers to a patient with a fasting plasma glucose concentration of greater than about 7.0 mmoL/L and a fasting C-peptide level of about, or less than about 0.4 nmoL/L. The term “type 2 diabetic” or “type 2 diabetes” generally refers to a patient with a fasting plasma glucose concentration of greater than about 7.0 mmoL/L and fasting C-peptide level that is within or higher than the normal physiological range of C-peptide levels (about 0.47 to 2.5 nmoL/L). It will be appreciated that a patient initially diagnosed as a type 2 diabetic may subsequently develop insulin-dependent diabetes, and may remain diagnosed as a type 2 patient, even though their C-peptide levels drop to those of a type 1.5 or type 1 diabetic patient (<0.2 nmol/L).

The terms “insulin-dependent patient” or “insulin-dependent diabetes” encompass all forms of diabetics/diabetes who/that require insulin administration to adequately maintain normal glucose levels unless specified otherwise.

Diabetes is frequently diagnosed by measuring fasting blood glucose, insulin, or glycated hemoglobin levels (which are typically referred to as hemoglobin A1c, Hb_{1c}, Hb_{A1c}, or A1C). Normal adult glucose levels are 60-126 mg/dL. Normal insulin levels are 30-60 pmoL/L. Normal HbA1c levels are generally less than 6%. The World Health Organization defines the diagnostic value of fasting plasma glucose concentration to 7.0 mmoL/L (126 mg/dL) and above for diabetes mellitus (whole blood 6.1 mmoL/L or 110 mg/dL), or 2-hour glucose level greater than or equal to 11.1 mmoL/L (greater than or equal to 200 mg/dL). Other values suggestive of or indicating high risk for diabetes mellitus include elevated arterial pressure greater than or equal to 140/90 mm Hg; elevated plasma triglycerides (greater than or equal to 1.7 mmoL/L [150 mg/dL]) and/or low HDL-cholesterol (less than 0.9 mmoL/L [35 mg/dL] for men; and less than 1.0 mmoL/L [39 mg/dL] for women); central obesity (BMI exceeding 30 kg/m²); microalbuminuria, where the urinary albumin excretion rate is greater than or equal to 20 μg/min or the albumin creatinine ratio is greater than or equal to 30 mg/g.

The term “delivery agent” refers to carrier compounds or carrier molecules that are effective in the oral delivery of therapeutic agents, and may be used interchangeably with “carrier”.

The term “homology” describes a mathematically-based comparison of sequence similarities which is used to identify genes or proteins with similar functions or motifs. The nucleic acid and protein sequences of the present invention can be used as a “query sequence” to perform a search against public databases to, e.g., identify other family members, related sequences, or homologs. Such searches can be performed using the NBLAST and XBLAST programs (version 2.0) of Altschul et al.: J. Mol. Biol. 215: 403-410, (1990). BLAST nucleotide searches can be performed with the NBLAST program, score=100, wordlength=12 to obtain nucleotide sequences homologous to nucleic acid molecules of the invention. BLAST protein searches can be performed with the XBLAST program, score=50, wordlength=3 to obtain amino acid sequences homologous to protein molecules of the invention. To obtain gapped alignments for comparison purposes, Gapped BLAST can be utilized as described in Altschul et al.: Nucleic Acids Res. 25(17): 3389-3402, (1997). When utilizing BLAST and Gapped BLAST programs, the default parameters of the respective programs (e.g., XBLAST and BLAST) can be used (see www.ncbi.nlm.nih.gov).

The term “homologous” refers to the relationship between two proteins that possess a “common evolutionary origin”, including proteins from superfamilies (e.g., the immunoglobulin superfamily) in the same species of animal, as well as homologous proteins from different species of animal (e.g., myosin light chain polypeptide; see Reeck et al.: Cell 50: 667, (1987)). Such proteins (and their encoding nucleic acids) have sequence homology, as reflected by their sequence similarity, whether in terms of percent identity or by the presence of specific residues or motifs and conserved positions. In specific embodiments, two nucleic acid sequences are “substantially homologous” or “substantially similar” when at least about 85%, and more preferably at least about 90% or at least about 95% of the nucleotides match over a defined length of the nucleic acid sequences, as determined by a sequence comparison algorithm known such as BLAST, FASTA, DNA Strider, CLUSTAL, etc. An example of such a sequence is an allelic or species variant of the specific genes of the present invention. Sequences that are substantially homologous may also be identified by hybridization, e.g., in a Southern hybridization experiment under, e.g., stringent conditions as defined for that particular system.

Similarly, in particular embodiments of the invention, two amino acid sequences are “substantially homologous” or “substantially similar” when greater than 80% of the amino acid residues are identical, or when greater than about 90% of the amino acid residues are similar (i.e., are functionally identical). Preferably the similar or homologous polypeptide sequences are identified by alignment using, e.g., the GCG (Genetics Computer Group, version 7, Madison, Wis.) pileup program, or using any of the programs and algorithms described above. The program may use the local homology algorithm of Smith and Waterman with the default values: gap creation penalty=−(1+⅓k), k being the gap extension number, average match=1, average mismatch=−0.333.

As used herein, “identity” means the percentage of identical nucleotide or amino acid residues at corresponding positions in two or more sequences when the sequences are aligned to maximize sequence matching, i.e., taking into account gaps and insertions. Identity can be readily calculated by known methods, including but not limited to those described in Computational Molecular Biology, Lesk A M, Ed., Oxford University Press, New York, (1988); Biocomputing: Informatics and Genome Projects, Smith D W, Ed., Academic Press, New York, (1993); Computer Analysis of Sequence Data, Part I, Griffin A M and Griffin H G, Eds., Humana Press, New Jersey, (1994); Sequence Analysis in Molecular Biology, von Heinje G, Academic Press, (1987); and Sequence Analysis Primer, Gribskov M and Devereux J, Eds., M Stockton Press, New York, (1991); and Carillo H and Lipman D, SIAM J. Applied Math., 48: 1073 (1988). Methods to determine identity are designed to give the largest match between the sequences tested. Moreover, methods to determine identity are codified in publicly available computer programs. Computer program methods to determine identity between two sequences include, but are not limited to, the GCG program package (Devereux J et al.: Nucleic Acids Res. 12(1): 387, (1984)), BLASTP, BLASTN, and FASTA (Altschul S F et al.: J. Molec. Biol. 215: 403-410, (1990) and Altschul S F et al.: Nucleic Acids Res. 25: 3389-3402, (1997)). The BLAST X program is publicly available from NCBI and other sources (BLAST Manual, Altschul S F et al., NCBI NLM NIH Bethesda, Md. 20894; Altschul S F et al., J. Mol. Biol. 215: 403-410, (1990)). The well-known Smith Waterman algorithm (Smith T F, Waterman M S: J. Mol. Biol. 147(1): 195-197, (1981)) can also be used to determine similarity between sequences.

The term “insulin” includes all forms of insulin including, without limitation, rapid-acting forms, such as Insulin Lispro rDNA origin: HUMALOG (1.5 mL, 10 mL, Eli Lilly and Company, Indianapolis, Ind.), Insulin Injection (Regular Insulin) from beef and pork (regular ILETIN I, Eli Lilly), human: rDNA: HUMULIN R (Eli Lilly), NOVOLIN R (Novo Nordisk, New York, N.Y.), Semi synthetic: VELOSULIN Human (Novo Nordisk), rDNA Human, Buffered: VELOSULIN BR, pork: regular Insulin (Novo Nordisk), purified pork: Pork Regular ILETIN II (Eli Lilly), Regular Purified Pork Insulin (Novo Nordisk), and Regular (Concentrated) ILETIN II U-500 (500 units/mL, Eli Lilly); intermediate-acting forms such as Insulin Zinc Suspension, beef and pork: LENTE ILETIN G I (Eli Lilly), Human, rDNA: HUMULIN L (Eli Lilly), NOVOLIN L (Novo Nordisk), purified pork: LENTE ILETIN II (Eli Lilly), Isophane Insulin Suspension (NPH): beef and pork: NPH ILETIN I (Eli Lilly), Human, rDNA: HUMULIN N (Eli Lilly), Novolin N (Novo Nordisk), purified pork: Pork NPH Eetin II (Eli Lilly), NPH-N (Novo Nordisk); and long-acting forms such as Insulin zinc suspension, extended (ULTRALENTE, Eli Lilly), human, rDNA: HUMULIN U (Eli Lilly).

The terms “measuring” or “measurement” mean assessing the presence, absence, quantity, or amount (which can be an effective amount) of either a given substance within a clinical- or patient-derived sample, including the derivation of qualitative or quantitative concentration levels of such substances, or otherwise evaluating the values or categorization of a patient's clinical parameters.

The term “meal” as used herein means a standard and/or a mixed meal.

The term “mean”, when preceding a pharmacokinetic value (e.g., mean tₘₐₓ), represents the arithmetic mean value of the pharmacokinetic value unless otherwise specified.

The term “mean baseline level” as used herein means the measurement, calculation, or level of a certain value that is used as a basis for comparison, which is the mean value over a statistically significant number of subjects, e.g., across a single clinical study or a combination of more than one clinical study.

The term “multiple dose” means that the patient has received at least two doses of the drug composition in accordance with the dosing interval for that composition.

The term “neuropathy” in the context of a “patient with neuropathy” or a patient that “has neuropathy”, means that the patient meets at least one of the four criteria outlined in the San Antonio Conference on diabetic neuropathy (report and recommendations of the San Antonio Conference on diabetic neuropathy. Ann. Neurol. 24 99-104 (1988)), which in brief include 1) clinical signs of polyneuropathy, 2) symptoms of nerve dysfunction, 3) nerve conduction deficits in at least two nerves, or 4) quantitative sensory deficits. The term “established neuropathy” means that the patient meets at least two of the four criteria outlined in the San Antonio Conference on diabetic neuropathy. The term “incipient neuropathy” refers to a patient that exhibits only nerve conduction deficits, and no other symptoms of neuropathy.

The term “normal glucose levels” is used interchangeably with the term “normoglycemic” and “normal” and refers to a fasting venous plasma glucose concentration of less than about 6.1 mmoL/L (110 mg/dL). Sustained glucose levels above normoglycemic are considered a pre-diabetic condition.

As used herein, the term “patient” in the context of the present invention is preferably a mammal. The mammal can be a human, non-human primate, mouse, rat, dog, cat, horse, or cow, but are not limited to these examples. Mammals other than humans can be advantageously used as patients that represent animal models of insulin-dependent diabetes mellitus, or diabetic conditions. A patient can be male or female. A patient can be one who has been previously diagnosed or identified as having insulin-dependent diabetes, or a diabetic condition, and optionally has already undergone, or is undergoing, a therapeutic intervention for the diabetes. A patient can also be one who is suffering from a long-term complication of diabetes. Preferably the patient is human.

The terms “PEG”, “polyethylene glycol”, or “poly(ethylene glycol)” as used herein refers to any water soluble poly(ethylene oxide), and includes molecules comprising the structure —(CH₂CH₂O)ₙ— where n is an integer from 2 to about 800. A commonly used PEG is end-capped PEG, wherein one end of the PEG is capped with a relatively inactive group such as an alkoxy while the other end is a hydroxyl group that may be further modified. An often-used capping group is methoxy and the corresponding end-capped PEG is often denoted mPEG. The notion PEG is often used instead of mPEG. Specific PEG forms of the invention are branched, linear, forked PEGs, and the like and the PEG groups are typically polydisperse, possessing a low polydispersity index of less than about 1.05. The PEG moieties of the invention will for a given molecular weight will typically consist of a range of ethylene glycol (or ethyleneoxide) monomers. For example, A PEG moiety of molecular weight 2000 Da will typically consist of 43±10 monomers, the average being around 43 monomers. The term “PEGylated” refers to the covalent attachment of PEG to another molecule, such as C-peptide.

The term “replacement dose” in the context of a replacement therapy for C-peptide refers to a dose of C-peptide or conjugated C-peptide that maintains C-peptide or conjugated C-peptide levels in the blood within a desirable range, particularly at a level which is at or above the minimum effective therapeutic level. In certain aspects, the replacement dose maintains the average steady-state concentration C-peptide or conjugated C-peptide levels above a minimum level of about 0.1 nM between dosing intervals. In certain aspects, the replacement dose maintains the average steady-state concentration C-peptide or conjugated C-peptide levels above a minimum level of about 0.2 nM between dosing intervals. In certain aspects, the replacement dose maintains the average steady-state concentration C-peptide or conjugated C-peptide levels above a minimum level of about 0.4 nM between dosing intervals.

The terms “subcutaneous” or “subcutaneously” or “S.C.” in reference to a mode of administration of insulin or conjugated C-peptide, refers to a drug that is administered as a bolus injection, or via an implantable device into the area in, or below the subcutis, the layer of skin directly below the dermis and epidermis, collectively referred to as the cutis. Preferred sites for subcutaneous administration and/or implantation include the outer area of the upper arm, just above and below the waist, except the area right around the navel (a 2-inch circle). The upper area of the buttock, just behind the hipbone. The front of the thigh, midway to the outer side, 4 inches below the top of the thigh to 4 inches above the knee.

The term “single dose” means that the patient has received a single dose of the drug composition or that the repeated single doses have been administered with washout periods in between. Unless specifically designated as “single dose” or at “steady-state” the pharmacokinetic parameters disclosed and claimed herein encompass both single-dose and multiple-dose conditions.

The term “sequence similarity” refers to the degree of identity or correspondence between nucleic acid or amino acid sequences that may or may not share a common evolutionary origin (see Reeck et al., supra). However, in common usage and in the present application, the term “homologous”, when modified with an adverb such as “highly”, may refer to sequence similarity and may or may not relate to a common evolutionary origin.

By “statistically significant”, it is meant that the result was unlikely to have occurred by chance. Statistical significance can be determined by any method known in the art. Commonly used measures of significance include the p-value, which is the frequency or probability with which the observed event would occur, if the null hypothesis were true. If the obtained p-value is smaller than the significance level, then the null hypothesis is rejected. In simple cases, the significance level is defined at a p-value of 0.05 or less.

As defined herein, the terms “sustained release”, “extended release”, or “depot formulation” refers to the release of a drug such as conjugated C-peptide from the sustained release composition or sustained release device which occurs over a period which is longer than that period during which the drug would be available following direct I.V. or S.C. administration of a single dose of drug. In one aspect, sustained release will be a release that occurs over a period of at least about one to two weeks, about two to four weeks, about one to two months, about two to three months, or about three to six months. In certain aspects, sustained release will be a release that occurs over a period of about six months to about one year. The continuity of release and level of release can be affected by the type of sustained release device (e.g., programmable pump or osmotically-driven pump) or sustained release composition, and type of conjugated C-peptides used (e.g., monomer ratios, molecular weight, block composition, and varying combinations of polymers), polypeptide loading, and/or selection of excipients to produce the desired effect, as more fully described herein.

Various sustained release profiles can be provided in accordance with any of the methods of the present invention. “Sustained release profile” means a release profile in which less than 50% of the total release of drug that occurs over the course of implantation/insertion or other method of administering the drug in the body occurs within the first 24 hours of administration. In a preferred embodiment of the present invention, the extended release profile is selected from the group consisting of; a) the 50% release point occurring at a time that is between 48 and 72 hours after implantation/insertion or other method of administration; b) the 50% release point occurring at a time that is between 72 and 96 hours after implantation/insertion or other method of administration; c) the 50% release point occurring at a time that is between 96 and 110 hours after implantation/insertion or other method of administration; d) the 50% release point occurring at a time that is between 1 and 2 weeks after implantation/insertion or other method of administration; e) the 50% release point occurring at a time that is between 2 and 4 weeks after implantation/insertion or other method of administration; f) the 50% release point occurring at a time that is between 4 and 8 weeks after implantation/insertion or other method of administration; g) the 50% release point occurring at a time that is between 8 and 16 weeks after implantation/insertion or other method of administration; h) the 50% release point occurring at a time that is between 16 and 52 weeks (1 year) after implantation/insertion or other method of administration; and i) the 50% release point occurring at a time that is between 52 and 104 weeks after implantation/insertion or other method of administration.

Additionally, use of a sustained release composition can reduce the “degree of fluctuation” (“DFL”) of the drugs plasma concentration. DFL is a measurement of how much the plasma levels of a drug vary over the course of a dosing interval (Cₘₐₓ−Cₘ₁ₙ/Cₘᵢₙ). For simple cases, such as I.V. administration, fluctuation is determined by the relationship between the elimination half-life (T_{1/2}) and dosing interval. If the dosing interval is equal to the half-life then the trough concentration is exactly half of the peak concentration, and the degree of fluctuation is 100%. Thus a sustained release composition with a reduced DFL (for the same dosing interval) signifies that the difference in peak and trough plasma levels has been reduced. Preferably, the patients receiving a sustained release composition of conjugated C-peptide have a DFL approximately 50%, 40%, or 30% of the DFL in patients receiving a non-extended release composition with the same dosing interval.

The terms “treating” or “treatment” means to relieve, alleviate, delay, reduce, reverse, improve, manage, or prevent at least one symptom of a condition in a patient. The term “treating” may also mean to arrest, delay the onset (i.e., the period prior to clinical manifestation of a disease), and/or reduce the risk of developing or worsening a condition.

As used herein, the terms “therapeutically effective amount”, “prophylactically effective amount”, or “diagnostically effective amount” is the amount of the drug, e.g., insulin or conjugated C-peptide, needed to elicit the desired biological response following administration.

The term “unit-dose forms” refers to physically discrete units suitable for human and animal patients and packaged individually as is known in the art. It is contemplated for purposes of the present invention that dosage forms of the present invention comprising therapeutically effective amounts of drug may include one or more unit doses (e.g., tablets, capsules, powders, semisolids [e.g., gelcaps or films], liquids for oral administration, ampoules or vials for injection, loaded syringes) to achieve the therapeutic effect. It is further contemplated for the purposes of the present invention that a preferred embodiment of the dosage form is a subcutaneously injectable dosage form.

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviations, per practice in the art. Alternatively, “about” with respect to the compositions can mean plus or minus a range of up to 20%, preferably up to 10%, more preferably up to 5%.

As used herein and in the appended claims, the singular forms “a”, “an”, and “the” include plural referents unless the context clearly indicates otherwise. Thus, for example, reference to “a molecule” includes one or more of such molecules, “a reagent” includes one or more of such different reagents, reference to “an antibody” includes one or more of such different antibodies, and reference to “the method” includes reference to equivalent steps and methods known to those of ordinary skill in the art that could be modified or substituted for the methods and pharmaceutical compositions described herein.

Unless defined otherwise, all technical and scientific terms used herein have the same meaning as commonly understood by one of ordinary skill in the art to which the invention belongs. The following abbreviations listed in Table A are used in certain sections of the disclosure:

TABLE A
LIST OF ABBREVIATIONS

| | |
|---|---|
| ADA | Anti-drug antibody |
| AUC | Area under the curve |
| AUC₍₀₋₇₎ | Area under the plasma concentration-time curve from |
| | time zero to Day 7 |
| AUC₍₀₋₁₄₎ | Area under the plasma concentration-time curve from |
| | time zero to Day 14 |
| AUC₍₀₋ₜ₎/ | Area under the plasma concentration-time curve from |
| AUCₜₐᵤ | time zero to the time of the last quantifiable concentration |
| AUC_{(0-inf)}/ | Area under the plasma concentration-time curve from |
| AUC_{inf} | time zero to infinity |
| Conc. | Concentration |
| Cₛₛ | Concentration at steady state |
| CL/F | Apparent clearance uncorrected for bioavailability (F) |
| CLₛₛ/F | Apparent clearance uncorrected for bioavailability (F) at |
| | steady state |
| Cₘₐₓ | Maximum observed concentration |
| ELISA | Enzyme-linked immunosorbent assay |
| F | Bioavailability or female |
| Fᵣₑₗ | Relative bioavailability |
| GLP | Good Laboratory Practice |
| h | Hours |
| i.v. | Intravenous |
| kg | Kilogram |
| L | Liter |
| M | Male |
| mg | Milligram |
| mL | Milliliter |
| min | Minutes |
| MTD | maximum tolerated dose |
| ND | Not determined |
| ng | Nanogram |
| NOEL | no observed effect level. |
| nM/ | Nanomolar |
| nmol/L | |
| nnol | Nanomole |
| QC | Quality control |
| PEG | Polyethylene glycol |
| RIA | Radioimmunoassay |
| s.c./S.C. | Subcutaneous |
| SD | Standard deviation |
| T_{1/2} | Terminal elimination half-life |
| Tₘₐₓ | Time to reach Cₘₐₓ |
| Vd/F | Apparent volume of distribution following subcutaneous |
| | administration, uncorrected for bioavailability (F) |
| Vdₛₛ/F | Apparent volume of distribution following subcutaneous |
| | administration, uncorrected for bioavailability (F) at |
| | steady state |
| wk | Week |

Although any methods, compositions, reagents, cells, similar or equivalent to those described herein can be used in the practice or testing of the invention, the preferred methods and materials are described herein.

All publications and references, including but not limited to patents and patent applications, cited in this specification are herein incorporated by reference in their entirety as if each individual publication or reference were specifically and individually indicated to be incorporated by reference herein as being fully set forth. Any patent application to which this application claims priority is also incorporated by reference herein in its entirety in the manner described above for publications and references.

The practice of the present invention will employ, unless otherwise indicated, conventional techniques of chemistry, molecular biology, microbiology, recombinant DNA and immunology, which are within the capabilities of a person of ordinary skill in the art. Such techniques are explained in the literature. See, for example, J. Sambrook, E. F. Fritsch, and T. Maniatis, 1989, Molecular Cloning: A Laboratory Manual, Second Edition, Books 1-3, Cold Spring Harbor Laboratory Press; Ausubel, F. M. et al. (1995 and periodic supplements; Current Protocols in Molecular Biology, ch. 9, 13, and 16, John Wiley & Sons, New York, N.Y.); B. Roe, J. Crabtree, and A. Kahn, 1996, DNA Isolation and Sequencing: Essential Techniques, John Wiley & Sons; J. M. Polak and James O′D. McGee, 1990, In Situ Hybridization: Principles and Practice; Oxford University Press; M. J. Gait (Editor), 1984, Oligonucleotide Synthesis: A Practical Approach, Irl Press; D. M. J. Lilley and J. E. Dahlberg, 1992, Methods of Enzymology: DNA Structure Part A: Synthesis and Physical Analysis of DNA Methods in Enzymology, Academic Press; Handbook of Drug Screening, edited by Ramakrishna Seethala, Prabhavathi B. Fernandes (2001, New York, N.Y., Marcel Dekker, ISBN O-8247-0562-9); Lab Ref: A Handbook of Recipes, Reagents, and Other Reference Tools for Use at the Bench, edited by Jane Roskams and Linda Rodgers, 2002, Cold Spring Harbor Laboratory, ISBN O-87969-630-3; Harris, J M, and Zalipsky, S, eds, Poly(ethylene glycol), Chemistry and Biological Applications, ACS, Washington, 1997; Veronese, F., and J. M. Harris, Eds., Peptide and protein PEGylation, Advanced Drug Delivery Reviews, 54(4) 453-609 (2002); Zalipsky, S., et al., “Use of functionalized Poly(Ethylene Glycols) for modification of polypeptides” in Polyethylene Glycol Chemistry: Biotechnical and Biomedical Applications. Each of these general texts is herein incorporated by reference.

The publications discussed above are provided solely for their disclosure before the filing date of the present application. Nothing herein is to be construed as an admission that the invention is not entitled to antedate such disclosure by virtue of prior invention.

### I. Polyethylene Glycol (PEG)

PEG is a well-known polymer with good solubility in many aqueous and organic solvents, which exhibits low toxicity, lack of immunogenicity, and is clear, colorless, odorless, and stable. For these reasons and others, PEG has been selected as the preferred polymer for attachment, but it has been employed solely for purposes of illustration and not limitation. Similar products may be obtained with other water-soluble polymers, including without limitation; polyvinyl alcohol, other poly(alkylene oxides) such as poly(propylene glycol) and the like, poly(oxyethylated polyols) such as poly(oxyethylated glycerol) and the like, carboxymethylcellulose, dextran, polyvinyl alcohol, polyvinyl purrolidone, poly-1,3-dioxolane, poly-I,3,6-trioxane, ethylene/maleic anhydride, and polyaminoacids. One skilled in the art will be able to select the desired polymer based on the desired dosage, circulation time, resistance to proteolysis, and other considerations.

Representative polymeric reagents and methods for conjugating such polymers to an active moiety are described in Harris, J. M. and Zalipsky, S., Eds, Poly(ethylene glycol), Chemistry and Biological Applications, ACS, Washington, 1997; Veronese, F., and J. M. Harris, Eds., Peptide and Protein PEGylation, Advanced Drug Delivery Reviews, 54(4); 453-609 (2002); Zalipsky, S., et al., “Use of Functionalized Poly Ethylene Glycols) for Modification of Polypeptides” in Polyethylene Glycol Chemistry: Biotechnical and Biomedical Applications, J. M. Harris, ed., Plenus Press, New York (1992); Zalipsky (1995) Advanced Drug Reviews 16:157-182; and in Roberts et al., Adv. Drug Delivery Reviews, 54, 459-476 (2002).

A wide variety of PEG derivatives are both commercially available and suitable for use in the preparation of the PEG-conjugates of the invention. For example, NOF Corp.'s SUNBRIGHT® Series (www.peg-drug.com) provides numerous PEG derivatives, including methoxypolyethylene glycols and activated PEG derivatives such as succinimidyl ester, methoxy-PEG amines, maleimides, and carboxylic acids, for coupling by various methods to C-peptide and Nektar Therapeutics' Advanced PEGylation also offers diverse PEG-coupling technologies to improve the safety and efficacy of therapeutics. Additional PEGs for use in forming a C-peptide conjugate of the invention include those available from Polypure (Norway), from QuantaBioDesign LTD (Ohio) and Sunbio, Inc (South Korea). Further PEG reagents suitable for use in forming a conjugate of the invention, and methods of conjugation are described in the Pasut. G., et al., Expert Opin. Ther. Patents (2004), 14(6) 859-893.

A search of patents, published patent applications, and related publications will also provide those skilled in the art reading this disclosure with significant possible PEG-coupling technologies and PEG-derivatives. For example, U.S. Pat. Nos. 7,026,440; 6,858,736; 6,828,401; 6,602,498; 6,495,659; 6,448,369, 6,436,386; 5,990,237; 5,932,462; 5,900,461; 5,824,784; 5,739,208; 5,672,662; 5,650,234; 5,629,384; 5,252,714; and 4,904,584; the contents of which are incorporated by reference in their entirety, describe such technologies and derivatives, and methods for their manufacture.

The PEGylated C-peptides according to the invention have PEG moieties with a molecular weight varying within a range of about 4,000 Da to 80,000 Da. The molecular weight ranges will typically be from about 4000 Da to about 10,000 Da, from about 10,000 Da to about 20,000 Da, from about 20,000 Da to about 30,000 Da, from about 30,000 Da to about 40,000 Da, from about 40,000 Da to about 50,000 Da, from about 50,000 Da to about 60,000 Da, from about 60,000 Da to about 70,000 Da, and from about 70,000 Da to about 80,000 Da. Non-limiting examples of average molecular weights of the PEG moieties are about 10,000 Da, about 20,000 Da, about 30,000 Da, about 40,000 Da, about 50,000 Da, about 60,000 Da, about 70,000 Da, and about 80,000 Da.

Because virtually all PEG polymers exist as mixtures of diverse high molecular mass, PEG molecular weight (MW) is typically reported as number average (Mₙ), weight average (M_{w}), or z-average (M_{z}) molecular weights. The weight average is probably the most useful of the three, because it fairly accounts for the contributions of different sized chains to the overall behavior of the polymer, and correlates best with most of the physical properties of interest.

$$Number average Mw ( Mn ) = \frac{Σ ( MiNi )}{ΣNi}$$

$$Weight average M W ( Mw ) = \\ Z average M W ( Mz ) = \frac{Σ ( {Mi}^{3} Ni )}{Σ ( {Mi}^{2} Ni )}$$

where “Ni” is the mole-fraction (or the number-fraction) of molecules with molecular weight “Mi” in the polymer mixture. The ratio of Mw to Mn is known as the polydispersity index (PDI), and provides a rough indication of the breadth of the distribution. The PDI approaches 1.0 (the lower limit) for special polymers with very narrow MW distributions.

The PEG groups of the invention will for a given molecular weight typically consist of a range of ethylene glycol (or ethyleneoxide; OCH₂CH₂) monomers. For example, a PEG group of molecular weight 2000 Da will typically consist of 43±10 monomers, the average being around 43-44 monomers.

The PEG groups of the present invention will typically comprise a number of subunits, e.g., each n, n₁ or n₂ or n₃ in any of the claimed compounds may each independently be from about 1 to about 1000, from about 1 to about 800, from about 1 to about 600, from about 1 to about 400, from about 1 to about 300, from about 1 to about 200. Well-suited PEG groups are such wherein the number of subunits (i.e. n₁, n₂, and n₃) are independently selected from the group consisting of from about 800 to about 1000; from about 800 to about 950; from about 600 to about 850; from about 400 to about 650; from about 200 to about 450, from about 180 to about 350; from about 100 to about 150; from about 35 to about 55; from about 42 to about 62; from about 12 to about 25 subunits, from about 1 to 10 subunits. In certain embodiments the PEGylated C-peptide will have a molecular weight of about 40 kDa, and thus n₁ and n₂ for each PEG chain in the branch chain PEGs will be within the range of about 440 to about 550, or about 450 to about 520.

Branched versions of the PEG polymer (e.g., a branched 40,000 Da PEG polymer comprised of two or more 10,000 Da to 20,000 Da PEG polymers or the like) having a total molecular weight of any of the foregoing can also be used.

Representative branched polymers described therein include those having the following generalized structure: (PEG)y-[Core]-[Linker];

where “[Core]” is a central or core molecule from which extends 2 or more PEG arms, the variable “y” represents the number of PEG arms, and “[Linker]” represents an optional linking moiety (as further defined below) that typically couples the [Core] to the C-peptide. In one alternative embodiment of the branched chain PEGs, at least one polymer arm possesses a terminal functional group suitable (e.g. NHS moiety) for reaction with C-peptide. Typically the branched chain polymers of the invention are coupled to the N-terminal amino group of the C-peptide.

In yet further embodiments the linker moiety can represent either a hydrolytically stable, or alternatively, a degradable linker, meaning that the linkage can be hydrolyzed under physiological conditions, e.g., the linkage comprises an ester, hydrolysable carbamate, carbonate, or other such group. Hydrolytically degradable linkages, useful not only as a degradable linkage within a polymer backbone, but also, in the case of certain embodiments of the invention, for covalently attaching a water-soluble polymer to a C-peptide, include: carbonate; imine resulting, for example, from reaction of an amine and an aldehyde (see, e.g., Ouchi et al. (1997) Polymer Preprints 38(I):582-3); phosphate ester, formed, for example, by reacting an alcohol with a phosphate group; hydrazone, e.g., formed by reaction of a hydrazide and an aldehyde; acetal, e.g., formed by reaction of an aldehyde and an alcohol; orthoester, formed, for example, by reaction between a formate and an alcohol; and esters, and certain urethane (carbamate) linkages. Illustrative PEG reagents for use in preparing a releasable C-peptide conjugate in accordance with the invention are described in U.S. Pat. Nos. 6,348,558, 5,612,460, 5,840,900, 5,880,131, and 6,376,470. Typically releasable linkers may be attached to any residue in C-peptide, and are not restricted to the N-terminal amino acid.

Branched PEGs such as those represented generally by the formula, (PEG)y-[Core]-[Linker], above can possess 2 polymer arms to about 8 polymer arms (i.e., “y” ranges from 2 to about 8). Preferably, such branched PEGs typically possess from 2 to about 4 polymer arms, Multi-armed polymers include those having 2, 3, 4, 5, 6, 7 or 8 PEG arms.

Core molecules in branched PEGs as described above include polyols, which are then further functionalized. Such polyols include aliphatic polyols having from 1 to 10 carbon atoms and from 1 to 10 hydroxyl groups, including ethylene glycol, alkane diols, alkyl glycols, alkylidene alkyl diols, alkyl cycloalkane diols, 1,5-decalindiol, 4,8-bis(hydroxymethyl)tricyclodecane, cycloalkylidene diols, dihydroxyalkanes, trihydroxyalkanes, and the like. Cycloaliphatic polyols may also be employed, including straight chained or closed-ring sugars and sugar alcohols, such as mannitol, sorbitol, inositol, xylitol, quebrachitol, threitol, arabitol, erythritol, adonitol, ducitol, facose, ribose, arabinose, xylose, lyxose, rhamnose, galactose, glucose, fructose, sorbose, mannose, pyranose, altrose, talose, tagitose, pyranosides, sucrose, lactose, maltose, and the like. Additional aliphatic polyols include derivatives of glyceraldehyde, glucose, ribose, mannose, galactose, and related stereoisomers. Other core polyols that may be used include crown ether, cyclodextrins, dextrins and other carbohydrates such as starches and amylose. Typical polyols include glycerol, pentaerythritol, sorbitol, and trimethylolpropane. Other suitable cores include lysine, and other polyamines, and PEG moieties comprising multiple functional terminal end groups.

Illustrative multi-armed PEGs having 2 arms, 3 arms, 4 arms, and 8 arms are known in the art, and are available commercially and/or can be prepared following techniques known to those skilled in the art. (See generally Pasut et al., (2004) Protein, peptide and non-peptide drug PEGylation for therapeutic application Expert Opinin. Ther. Patents 14(6) 859-894). Additional branched-PEGs for use in forming a C-peptide conjugate of the present invention include those described in U.S. Patent Application Publication Nos. 20050009988, 20060194940, 20090234070, 20070031371, U.S. Pat. Nos. 6,664,331; 6,362,254; 6,437,025; 6,541,543; 6,664,331; 6,730,334; 6,774,180; 6,838,528; 7,030,278; 7,026,440; 7,053,150; 7,157,546; 7,223,803; 7,265,186; 7,419,600; 7,432,330; 7,432,331; 7,511,094; 7,528,202; 7,589,157; and PCT publication numbers WO2005000360, WO2005108463, WO2005107815, WO2005028539 and WO200605108463.

Those of ordinary skill in the art will recognize that the foregoing discussion describing linear and branched chain PEGs for use in forming a C-peptide conjugate is by no means exhaustive and is merely illustrative, and that all polymeric materials, and branched PEG structures having the qualities described herein are contemplated. Moreover, based on the instant invention, one of ordinary skill in the art can readily determine the appropriate size and optimal structure of alternative PEGylated C-peptides using routine experimentation, for example, by obtaining the clearance profile for each conjugate by administering the conjugate to a patient and taking periodic blood and/or urine samples, as described herein. Once a series of clearance profiles has been obtained for each tested conjugate, a conjugate or mixture of conjugates, having the desired clearance profile(s) can be determined.

### II. PEG Linker Moieties

The particular linkage between the C-peptide and the water-soluble polymer depends on a number of factors, including the desired stability of the linkage, its hydrophobicity, the particular linkage chemistry employed, and impact on the aqueous solubility, and aggregation state of the PEGylated C-peptide. Exemplary linkages are hydrolytically stable, and water soluble, representative suitable linker can comprise any combination of amide, a urethane (also known as carbamate), amine, thioether (also known as sulfide), or urea (also known as carbamide) groups.

There are many commercially available examples of suitable water-soluble linker moieties and/or these can be prepared following techniques known to those skilled in the art.

In one embodiment of the PEGylated C-peptides of general formula (I)-(XIV) the PEGylated C-peptide comprises one or more linkers independently selected from;
—X—, —CO—, —(CH₂)ₘ₂—, —(CH₂)ₘ₁—CO—, —CO—(CH₂)ₘ₁—, —CO—X—CO—,
—(CH₂)ₘ₁—X—(CH₂)ₘ₁—, —(CH₂)ₘ₁—CO—(CH₂)ₘ₁—, —X—CO—X—, —X—(CH₂)ₘ₁—X—,
—CO—(CH₂)ₘ₁—CO—, —X—CO—(CH₂)ₘ₁—, —(CH₂)ₘ₁—CO—X—, —X—(CH₂)ₘ₁—CO—X—,
—X—CO—(CH₂)ₘ₁X—, —X—CO—(CH₂)ₘ₁—CO—X—(CH₂)ₘ₁—X—CO—,
—X—(CH₂)ₘ₁—X—CO—(CH₂)ₘ₂—,
—X—(CH₂)ₘ₁—CO—X—(CH₂)ₘ₂—,
—X—(CH₂)ₘ₁—X—CO—(CH₂)ₘ₂—X—,
—X—(CH₂)ₘ₁—X—CO—(CH₂)ₘ₂—CO—,
—X—(CH₂)ₘ₁—CO—X—(CH₂)ₘ₂—X—, and
—X—(CH₂)ₘ₁—CO—X—(CH₂)ₘ₂—CO—;

wherein;
each X is independently selected from —O—, —S—, or NH— or is missing;
each m₁ is independently 0 to 5; and
each m₂ is independently 1 to 5.

In another embodiment of the PEGylated C-peptides of formula (I) (II), (III), or (IV) the PEGylated C-peptide comprises one or more linkers independently selected from;
—X₁—(CH₂)ₘ₄—CO—;
—X₁—CO—;
—X₁—CO—(CH₂)ₘ₄—CO—;
—X₁—CO—X₂—(CH₂)ₘ₃—CO—; and
—X₁ (CH2)ₘ₂—X₂—CO—(CH₂)ₘ₄—CO—;

wherein;
X₁ is —O—, or missing;
X₂ is NH—;
m₂ is 1 to 5
m₃ is 2; and
m₄ is 1 to 5.

In another embodiment of the PEGylated C-peptides of formula (II), (III), or (IV) the PEGylated C-peptide comprises one or more linkers independently selected from;
—X₁—CO—X₂—(CH₂)ₘ₅—X₁—(CH₂—CH₂—O)ₙ₃—X—,
X₁—CO—X₂—(CH₂)ₘ₅—X₁—(CH₂—CH₂—O)ₙ₃—(CH₂)ₘ₅—CO—,
—X₁—CO—X₂—(CH₂)ₘ₅—X₁—(CH₂—CH₂—O)ₙ₃—CO—, and
—X₁—CO—X₂—(CH₂)ₘ₅—X₁—(CH₂—CH₂—O)ₙ₃—CO—(CH₂)ₘ₅—CO—;

wherein;
X is independently selected from —O—, —S—, or NH— or is missing;
X₁ is —O—, or missing;
X₂ is NH—;
each m₅ is independently selected from 1 to 5; and
each n₃ is independently selected from 1 to 400.

In another embodiment of the PEGylated C-peptides of formula (II), (III) or (IV), the PEGylated C-peptide comprises one or more linkers independently selected from;
—X₁CO—X₂—(CH₂)ₘ₅—X₁—(CH₂—CH₂—O)ₙ₃—(CH₂)ₘ₆—CO—,
—X₁—CO—X₂—(CH₂)ₘ₅—X₁—(CH₂—CH₂—O)ₙ₃—CO—, and
—X₁—CO—X₂—(CH₂)ₘ₅—X₁—(CH₂—CH₂—O)ₙ₃—CO—(CH₂)ₘ₇—CO—;

wherein;
X₁ is —O—, or is missing;
X₂ is NH—;
m₅ is 3;
m₆ is independently 2 or 5;
m₇ is 3; and
n₃ is 1 to 400.

In another embodiment of the PEGylated C-peptides of formula (IV), the PEGylated C-peptide comprises a linker independently selected from;
—X—, —CO—, —(CH₂)ₘ₂—, and —X₁—C(O)—X₂—;

wherein;
X is —O—, or —S—, or —NH— or is missing;
X₁ and X2 are independently selected from NH—; or —O—, or is
missing; and m₂ is independently 1 to 5.

Those of ordinary skill in the art will recognize that the foregoing discussion describing linker moieties for use in forming a C-peptide conjugate is by no means exhaustive and is merely illustrative, and that all linkers having the qualities described herein are contemplated.

Moreover, based on the teachings described herein, one of ordinary skill in the art can readily determine the appropriate size and optimal structure of the linker using routine experimentation. For example by testing a number of different commercially available PEG derivatives with different linker moieties and characterizing the biological activity, solubility and stability of the resulting PEGylated C-peptide.

### III. Activated Functional Groups and Reaction Conditions

The only natural free amino group in human C-peptide is the N-terminal amino group, and thus the selective conjugation of a polymeric PEG group to the N-terminal amino group of C-peptide can be readily accomplished using a variety of commercially available activated PEGs and standard coupling approaches.

In one approach, a C-peptide is conjugated to the PEG reagent via an activated functional group, such as an active ester such as a succinimidyl derivative (e.g., an N-hydroxysuccinimide ester (NHS)). In this approach, the PEG bearing the reactive ester is reacted with the C-peptide in aqueous media under appropriate pH conditions, at room temperature or 4° C., for a few hours to overnight. Typically the polymeric reagent is coupled to the activated functional group via a linker as described herein.

N-terminal PEGylation, with a PEG reagent bearing an N-hydroxysuccinimide ester (NHS group), is typically carried out at room temperature, or 4° C., in a polar aprotic solvent such as dimethylformamide (DMF) or acetonitrile, or a combination thereof (with small amounts of water to solubilize the peptide) under slightly basic pH conditions, e.g., from pHs ranging from about 7.5 to about 8. Reaction times are typically in the range of 1 to 24 hours, depending upon the pH and temperature of the reaction.

N-terminal PEGylation, with a PEG reagent bearing an aldehyde group, is typically conducted under mild conditions, in the presence of sodium cyanoborohydride (10 equiv.), 4° C., at pHs from about 5 to 10, for about 20 to 36 hours. N-terminal pegylation may be conducted, for example, in 100 mM sodium acetate or 100 mM sodium biphosphate buffer at pH 5.0˜6.0. The buffer may additionally contain 20 mM sodium cyanoborahydride. The molar ratio of compound to mPEG-aldehyde may be 1:5˜1:10. The pegylation is then stirred overnight at ambient or refrigeration temperature.

N-terminal PEGylation, with a PEG reagent bearing p-Nitrophenyloxycarbonyl group, is typically conducted with borate or phosphate buffer at pHs from about 8 to 8.3, at room temperature overnight.

For all the coupling reactions, varying ratios of polymeric reagent to C-peptide may be employed, e.g., from an equimolar ratio up to a 10-fold molar excess of polymer reagent. Typically, up to a 2-fold molar excess of polymer reagent will suffice. Exemplary activated PEGs include, e.g., those listed in Table 1. In the following list, selected PEGylation reagents are listed. Other active groups and linkers may be employed, and are known to those skilled in the art.

TABLE 1
Exemplary Activated PEGs

| Used to Prepare Representative Compounds | Abbreviation & Molecular |
|---|---|
| of Structural Formula (I)-(XIV) Through | Weight Range |
| Reaction With C-peptide | (in Da) |
| Structural Formula (I)-(II) | Thermo Scientific Pierce |
| | TMS(PEG)₁₂ MW = 2,400 |
| Structural Formula (III)-(V) | SUNBRIGHT ME-BE200CH- |
| | TS MW = 20,000 |
| | SUNBRIGHT ME-BE200CM- |
| | TS MW = 20,000 |
| | SUNBRIGHT ME-BE200EH1- |
| | TS MW = 20,000 |
| | SUNBRIGHT ME-BE200EH5- |
| | TS MW = 20,000 |
| Structural Formula (VI) | SUNBRIGHT OE-020CS |
| | MW = 2,000 |
| | SUNBRIGHT OE-040CS |
| | MW = 4,000 |
| | SUNBRIGHT OE-080CS |
| | MW = 8,000 |
| Structural Formula (VII) | SUNBRIGHT PTE2-200GS3 |
| | MW = 20,000 |
| | SUNBRIGHT PTE2-400GS3 |
| | MW = 40,000 |
| Structural Formula (VIII) | SUNBRIGHT XY4-200NP |
| | MW = 20,000 |
| | SUNBRIGHT XY4-400NP |
| | MW = 40,000 |
| | SUNBRIGHT XY4-200TS |
| | MW = 20,000 |
| | SUNBRIGHT XY4-400TS |
| | MW = 40,000 |
| | SUNBRIGHT XY4-200GS2 |
| | MW = 20,000 |
| | SUNBRIGHT XY4-400GS2 |
| | MW = 40,000 |
| | SUNBRIGHT XY4-200AL |
| | MW = 20,000 |
| | SUNBRIGHT XY4-400AL |
| | MW = 40,000 |
| Structural Formula (IX) | SUNBRIGHT GL4-400NP |
| | MW = 40,000 |
| | SUNBRIGHT GL4-600NP |
| | MW = 60,000 |
| | SUNBRIGHT GL4-800NP |
| | MW = 80,000 |
| | SUNBRIGHT GL4-400TS |
| | MW = 40,000 |
| | SUNBRIGHT GL4-600 TS |
| | MW = 60,000 |
| | SUNBRIGHT GL4-800 TS |
| | MW = 80,000 |
| | SUNBRIGHT GL4-400GS2 |
| | MW = 40,000 |
| | SUNBRIGHT GL4-600 GS2 |
| | MW = 60,000 |
| | SUNBRIGHT GL4-800 GS2 |
| | MW = 80,000 |
| | SUNBRIGHT GL4-400AL3 |
| | MW = 40,000 |
| | SUNBRIGHT GL4-600 AL3 |
| | MW = 60,000 |
| | SUNBRIGHT GL4-800 AL3 |
| | MW = 80,000 |
| Structural Formula (X)-(XI) | JenKem GLUC-NHS-35K |
| | MW = 35,000 |
| | JenKem GALA-NHS-35K |
| | MW = 35,000 |
| Structural Formula (XII) | JenKem Y-NHS-40K |
| | MW = 40,000 |
| | JenKem Y-MAL-40K |
| | MW = 40,000 |
| | JenKem Y-AALD-40K |
| | MW = 40,000 |
| | JenKem Y-PALD-40K |
| | MW = 40,000 |

The PEGylated C-peptide can be purified after neutralization of the reaction buffer, by any convenient approach, e.g., by precipitation with isopropyl-ether followed by reverse phase HPLC or ion exchange chromatography.

### IV. Acylated C-Peptides

The only natural free amino group in human C-peptide is the N-terminal amino group, and thus the selective conjugation of an acylating agent to the N-terminal amino group of C-peptide can be readily accomplished using a variety of activated carboxylic acids and standard coupling approaches to give compounds of structural Formula XIII.

In one approach, a C-peptide is conjugated to the acylating reagent via an activated functional group, such as an active ester such as a succinimidyl derivative (e.g., an N-hydroxysuccinimide ester (NHS)). In this approach, the acylating reagent bearing the reactive ester is reacted with the C-peptide in aqueous media under appropriate pH conditions, at room temperature or 4° C., for a few hours to overnight.

In one approach, a C-peptide is conjugated to the acylating reagent via an activated functional group preformed or formed in situ through the reaction of a carboxylic acid with a peptide coupling reagent. Exemplary peptide coupling agents include carbodiimides such as dicyclohexylcarbodiimide (DCC), diisopropylcarbodiimide (DIC), and ethyl-(N′,N′-dimethylamino)propylcarbodiimide hydrochloride (EDC); phosphonium-based reagents such as (benzotriazol-1-yloxy)tris(dimethylamino)phosphonium hexafluorophosphate (BOP), (benzotriazol-1-yloxy)tripyrrolidinophosphonium hexafluorophosphate, and Bromotripyrrolidinophosphonium hexafluorophosphate; aminium-based reagents such as O-(benzotriazol-1-yl)-N,N,N′,N′-tetramethyluronium hexafluorophosphate (HBTU), O-(benzotriazol-1-yl)-N,N,N′,N′-tetramethyluronium tetrafluoroborate (TBTU), and O-(7-azabenzotriazol-1-yl)-N,N,N′,N′-tetramethyluronium hexafluorophosphate (HATU); O-(6-chlorobenzotriazol-1-yl)-N,N,N′,N′-tetramethyluronium hexafluorophosphate (HCTU), and O-(3,4-dihydro-4-oxo-1,2,3-benzotriazine-3-yl)-N,N,N′,N′-tetramethyluronium tetrafluoroborate (TDBTU). In some cases, the carboxylic acid is reacted with the peptide coupling reagent in the presence of additives that allow the formation of an intermediate activated ester, such as N-hydroxysuccinimide, 1-hydroxy-benzotriazole (HOBt), 1-hydroxy-7-aza-benzotriazole (HOAt), and ethyl 2-cyano-2-(hydroxyimino)acetate. In this approach, the acylating reagent formed by reaction of the carboxylic acid bearing the activated functional group is reacted with the C-peptide in aqueous media, with an optional organic co-solvent, under appropriate pH conditions, at room temperature or 4° C., for a few hours to overnight.

N-terminal acylation, with an acylating reagent bearing an N-hydroxysuccinimide ester (NHS group), is typically carried out at room temperature, or 4° C., in a polar aprotic solvent such as dimethylformamide (DMF) or acetonitrile, or a combination thereof (with small amounts of water to solubilize the peptide) under slightly basic pH conditions, e.g., from pHs ranging from about 7.5 to about 8. Reaction times are typically in the range of 1 to 24 hours, depending upon the pH and temperature of the reaction.

N-terminal acylation, with an acylating reagent bearing a p-nitrophenyloxycarbonyl group, is typically conducted with borate or phosphate buffer at pHs from about 8 to 8.3, at room temperature overnight.

For all the coupling reactions, varying ratios of acylating reagent to C-peptide may be employed, e.g., from an equimolar ratio up to a 10-fold molar excess of polymer reagent. Typically, up to a 2-fold molar excess of polymer reagent will suffice.

### V. Human Serum Albumin-Conjugated C-Peptides

Human serum albumin is a water-soluble monomeric 67 kDa protein which is the most abundant protein in human plasma, constituting approximately half of the blood serum protein. Human serum albumin transports hormones, fatty acids, and other compounds, buffers pH, and maintains osmotic pressure, among other functions. Human serum albumin conjugates of therapeutic proteins have been investigated as a method of increasing half-life in plasma due to stabilization from enzymatic degradation or reduced elimination through the kidney. Conjugation occurs when a strategically placed reactive group on a therapeutic peptide reacts with a nucleophilic moeity of human serum albumin. The foremost human serum albumin nucleophile is the unpaired Cys34 thiol, which reacts with Michael acceptors such as a maleimido derivative, leading to new bioactive protein constructs.

Examples or protein-human serum albumin conjugates and representative methods of synthesis can be found in Thibaudeau et al., Bioconjugate Chem., 2005, 16, 1000-1008; Leger et al., Bioorganic & Medicinal Chemistry Letters, 2004, 14, 4395-4398; Leger et al., Bioorganic & Medicinal Chemistry Letters, 2004, 14, 841-845; Leger et al., Bioorganic & Medicinal Chemistry Letters, 2003, 13, 3571-3575; and Jette et al., Endocrinology, 2005, 146(7), 3052-3058, each of which are hereby incorporated by reference in their entireties.

C-Peptide can be conjugated to human serum albumin using various bifunctional linkers that attach to C-peptide at the N-terminal amino acid at one end and human serum albumin at the other. In certain embodiments, human serum albumin is conjugated through a linkage to the 34-cysteine. In further embodiments, the 34-cysteine of human serum albumin is attached to a linker containing a maleimide group.

In some cases, C-peptide is functionalized by acylation at the N-terminus using a bifunctional linker. Acylation of C-peptide can be readily accomplished using a variety of bifunctional carboxylic acids, such as 3-maleimidopropionic acid or 8-(3-maleimidopropionamido)octanoic acid, and standard peptide coupling or reductive amination approaches as described above to give a C-peptide modified with a maleimide group-containing linker. In an alternate approach, C-peptide is first modified by reacting it with a first linker such as N-Fmoc-2-[2-(2-aminoethoxyl)ethoxy]acetic acid, which is then deprotected and coupled to an additional bifunctional carboxylic acid such as 3-maleimidopropionic acid. In a further approach, C-peptide is reacted with sulfosuccinimidyl-4-(N-maleimidomethyl)cyclohexane-1-carboxylate (Sulfo-SMCC) and then the resulting functionalized C-peptide is reacted with human serum albumin in aqueous solution at pH 79.

Following functionalization of C-peptide with a linker, the functionalized C-peptide is then reacted with human serum albumin. In examples where a maleimide group-containing linker is used, the reaction with human serum albumin takes place in aqueous solution, optionally in the presence of a polar aprotic solvent such as dimethylformamide (DMF) or acetonitrile, or a combination thereof. The C-peptide-human serum albumin conjugate can then be purified through any technique known in the art, such as gel filtration chromatography.

Alternatively, human serum albumin conjugates of C-peptide can be expressed as a fusion protein, in which Contiguous cDNA for target protein/peptide with DNA encoding albumin produces a single protein.

### VI. Hydroxyethyl Starch-Conjugated C-Peptides

Hydroxyethyl starch is a water-soluble nonionic starch derivative. Intravenous solutions of hydroxyethyl starch are used to prevent shock following severe blood loss caused by trauma, surgery, or other causes, whereby the hydroxyethyl starch solution acts to increase the blood volume, allowing red blood cells to continue to deliver oxygen to the body.

Examples or protein-hydroxyethyl starch conjugates and representative methods of synthesis can be found in US 20110200555 and Besheer et al., Bioconjugation Protocols: Strategies and Methods, Methods in Molecular Biology, vol. 751, chapter 2, 17-27, each of which are hereby incorporated by reference in their entireties.

Hydroxyethylstarch can be modified through several techniques. First, terminal aldehyde groups can be modified by reaction with amino-oxy, hydrazine, or amine groups to form Schiff bases, which can optionally be reduced with reagent such as sodium cyanoborohydride. Alternatively, hydroxyethyl starch can be reacted with an oxidizing agent, such as periodate, to give a functionalized hydroxyethyl starch containing carboxylate groups, which can be coupled to a therapeutic peptide or a bifunctional linker using peptide coupling techniques known in the art. In a further alternative method, hydroxyethyl starch can be reached with a hydroxyl activating agent such as p-toluenesulfonyl chloride and then reacted with a nucleophillic linker. In some cases, the nucleophillic linker is a homobifunctional linker such as hexamethylenediamine. Once functionalised, the hydroxyethyl starch can be reacted with an appropriate linker and coupled to C-peptide using conjugation techniques described herein.

### VII. Therapeutic Forms of C-Peptide

The terms “C-peptide” or “proinsulin C-peptide” as used herein includes all naturally-occurring and synthetic forms of C-peptide that retain C-peptide activity. Such C-peptides include the human peptide, as well as peptides derived from other animal species and genera, preferably mammals. Preferably, “C-peptide” refers to human C-peptide having the amino acid sequence EAEDLQVGQVELGGGPGAGSLQPLALEGSLQ (SEQ. ID. No. 1 in Table 2).

In certain embodiments, “C-peptide” refers to the C-terminal pentapeptide sequence (EGSLQ) (SEQ. ID. No. 31), or other variants which retain the C-terminal pentapeptide sequence (EGSLQ) (SEQ. ID. No. 31) and retain substantially the same biological activity of naturally occuring human C-peptide.

C-peptides from a number of different species have been sequenced, and are known in the art to be at least partially functionally interchangeable. It would thus be a routine matter to select a variant being a C-peptide from a species or genus other than human. Several such variants of C-peptide (i.e., representative C-peptides from other species) are shown in Table 2 (see SEQ. ID. Nos. 1-29).

TABLE 2
C-peptide Variants

| | | |
|---|---|---|
| human M- | Human | gb\|AAA72531.1\| |
| proinsulin | EAEDLQVGQVELGGGPGAGSLQPLALEGS | dbj\|BAH59081.1\| |
| | LQ | |
| | (SEQ. ID. No. 1) | |
| Pan | (SEQ. ID. No. 1) | NP_001008996.1\| |
| troglodytes | EAEDLQVGQVELGGGPGAGSLQPLALE | emb\|CAA43403.1\| |
| | GSLQ | GENE ID: |
| | Alignment | 449570_INS |
| | EAEDLQVGQVELGGGPGAGSLQPLALEGS | |
| | LQ | |
| | (SEQ. ID. No. 2) | |
| | EAEDLQVGQVELGGGPGAGSLQPLALE | |
| | GSLQ | |
| | Identities = 31/31 (100%), | |
| | Positives = 31/31 (100%), | |
| | Gaps = 0/31 (0%) | |
| Gorilla | (SEQ. ID. No. 1) | gb\|AAN06935.1\| |
| gorilla | EAEDLQVGQVELGGGPGAGSLQPLALEGS | |
| | LQ | |
| | Alignment | |
| | EAEDLQVGQVELGGGPGAGSLQPLALEGS | |
| | LQ | |
| | (SEQ. ID. No. 3) | |
| | EAEDLQVGQVELGGGPGAGSLQPLALEGS | |
| | LQ | |
| | Identities = 31/31 (100%), | |
| | Positives = 31/31 | |
| | (100%), Gaps = 0/31 (0%) | |
| Pongo | (SEQ. ID. No. 1) | gb\|AAN06937.1\| |
| pygmaeus | EAEDLQVGQVELGGGPGAGSLQPLALEGS | |
| (Bornean | LQ | |
| orangutan) | EAEDLQVGQVELGGGPGAGSLQPLALEGS | |
| | LQ | |
| | (SEQ. ID. No. 4) | |
| | EAEDLQVGQVELGGGPGAGSLQPLALEGS | |
| | LQ | |
| | Identities = 31/31 (100%), | |
| | Positives = 31/31 | |
| | (100%), Gaps = 0/31 (0%) | |
| Chlorocebus | (SEQ. ID. No. 1) | emb\|CAA43405.1\| |
| aethiops | EAEDLQVGQVELGGGPGAGSLQPLALEGS | |
| (Monkey) | LQ | |
| | EAED | |
| | QVGQVELGGGPGAGSLQPLALEGSLQ | |
| | (SEQ. ID. No. 5) | |
| | EAEDPQVGQVELGGGPGAGSLQPLALEGS | |
| | LQ | |
| | Identities = 30/31 (96%), | |
| | Positives = 30/31 | |
| | (96%), Gaps = 0/31 (0%) | |
| Canis lupus | (SEQ. ID. No. 1) | ref\|NP_0011235 |
| familiaris | EAEDLQVGQVELGGGPGAGSLQPLALEGS | 65.1\|sp\|P01321. |
| (Dog) | LQ | 1\|INS_CANFAe |
| | E EDLQV VEL G PG G | mb\|CAA23475.1\| |
| | LQPLALEG + LQ | GENE ID: |
| | (SEQ. ID. No. 6) | 483665_INS |
| | EVEDLQVRDVELAGAPGEGGLQPLALEGA | |
| | LQ | |
| | Identities = 23/31 (74%), | |
| | Positives = 24/31 | |
| | (77%), Gaps = 0/31 (0%) | |
| Oryctolagus | (SEQ. ID. No. 1) | gb\|ACK44319.1\| |
| cuniculus | EAEDLQVGQVELGGGPGAGSLQPLALEGS | |
| (Rabbit) | LQ | |
| | E E + LQVGQ ELGGGP AG LQP | |
| | ALE + LQ | |
| | (SEQ. ID. No. 7) | |
| | EVEELQVGQAELGGGPDAGGLQPSALELA | |
| | LQ | |
| | Identities = 23/31 (74%), | |
| | Positives = 25/31 | |
| | (80%), Gaps = 0/31 (0%) | |
| Rattus | (SEQ. ID. No. 1) | ref\|NP_062003.1\| |
| norvegicus | EAEDLQVGQVELGGGPGAGSLQPLALEGS | sp\|P01323.1\|INS2_RAT |
| | LQ | emb\|CAA24560.1\| |
| | E ED QV Q + ELGGGPGAG LQ | GENE ID: 24506 |
| | LALE +Q | Ins2 |
| | (SEQ. ID. No. 8) | |
| | EVEDPQVAQLELGGGPGAGDLQTLALEVA | |
| | RQ | |
| | Identities = 22/31 (70%), | |
| | Positives = 24/31 | |
| | (77%), Gaps = 0/31 (0%) | |
| Apodemus | (SEQ. ID. No. 1) | gb\|ABB89748.1\| |
| semotus | EAEDLQVGQVELGGGPGAGSLQPLALEGS | |
| (Taiwan field | LQ | |
| mouse) | E ED QV Q + ELGGGPGAG LQ | |
| | LALE +Q | |
| | (SEQ. ID. No. 9) | |
| | EVEDPQVAQLELGGGPGAGDLQTLALEVA | |
| | RQ | |
| | Identities = 22/31 (70%), | |
| | Positives = 24/31 | |
| | (77%), Gaps = 0/31 (0%) | |
| Geodia | (SEQ. ID. No. 1) | pir\|\|S09278 |
| cydonium | EAEDLQVGQVELGGGPGAGSLQPLALEGS | |
| sponge | LQ | |
| | E ED QVGQVELG GPGAGS Q | |
| | LALE + Q | |
| | (SEQ. ID. No. 10) | |
| | EVEDPQVGQVELGAGPGAGSEQTLALEVA | |
| | RQ | |
| | Identities = 23/31 (74%), | |
| | Positives = 24/31 | |
| | (77%), Gaps = 0/31 (0%) | |
| Mus musculus | (SEQ. ID. No. 1) | ref\|NF_032413.1\| |
| | EAEDLQVGQVELGGGPGAGSLQPLALE | sp\|P01326.1\|INS |
| | E ED QV Q + ELGGGPGAG LQ | 2_MOUSEemb\|C |
| | LALE | AA28433.1\| |
| | (SEQ. ID. No. 11) | Ins2 |
| | EVEDPQVAQLELGGGPGAGDLQTLALE | |
| | GENE ID: 16334 | |
| | Identities = 21/27 (77%), | |
| | Positives = 22/27 | |
| | (81%), Gaps = 0/27 (0%) | |
| Mus caroli | (SEQ. ID. No. 1) | gb\|ABB89749.1\| |
| (Ryukyu | EAEDLQVGQVELGGGPGAGSLQPLALE | |
| mouse) | E ED QV Q + ELGGGPGAG LQ | |
| | LALE | |
| | (SEQ. ID. No. 12) | |
| | EVEDPQVAQLELGGGPGAGDLQTLALE | |
| | Identities = 21/27 (77%), | |
| | Positives = 22/27 | |
| | (81%), Gaps = 0/27 (0%) | |
| Rattus | (SEQ. ID. No. 1) | prf\|\|720460B |
| norvegicus | EAEDLQVGQVELGGGPGAGSLQPLALEGS | |
| | LQ | |
| | E ED QV Q + ELGGGPGAG LQ | |
| | LALE +Q | |
| | (SEQ. ID. No. 13) | |
| | EVEDPQVPQLELGGGPGAGDLQTLALEVA | |
| | RQ | |
| | Identities = 22/31 (70%), | |
| | Positives = 24/31 | |
| | (77%), Gaps = 0/31 (0%) | |
| Rattus losea | (SEQ. ID. No. 1) | gb\|ABB89747.1\| |
| | EAEDLQVGQVELGGGPGAGSLQPLALEGS | |
| | LQ | |
| | E ED QV Q ELGGGPGAG LQ | |
| | LALE +Q | |
| | (SEQ. ID. No. 14) | |
| | EVEDPQVAQQELGGGPGAGDLQTLALEVA | |
| | RQ | |
| | Identities = 22/31 (70%), | |
| | Positives = 23/31 | |
| | (74%), Gaps = 0/31 (0%) | |
| Niviventer | (SEQ. ID. No. 1) | gb\|ABB89750.1\| |
| coxingi | EAEDLQVGQVELGGGPGAGSLQPLALEGS | |
| (Coxing's | LQ | |
| white-bellied | E ED QV Q + ELGGGPG G LQ | |
| rat) | LALE + Q | |
| | (SEQ. ID. No. 15) | |
| | EVEDPQVPQLELGGGPGTGDLQTLALEVA | |
| | RQ | |
| | Identities = 21/31 (67%), | |
| | Positives = 23/31 | |
| | (74%), Gaps = 0/31 (0%) | |
| Microtus | (SEQ. ID. No. 1) | gb\|ABB89752.1\| |
| kikuchii | AEDLQVGQVELGGGPGAGSLQPLALE | |
| (Taiwan vole) | ED QV Q + ELGGGPGAG LQ | |
| | LALE | |
| | (SEQ. ID. No. 16) | |
| | VEDPQVAQLELGGGPGAGDLQTLALE | |
| | Identities = 20/26 (76%), | |
| | Positives = 21/26 | |
| | (80%), Gaps = 0/26 (0%) | |
| Rattus | (SEQ. ID. No. 1) | ref\|NP_062002.1\| |
| norvegicus | EAEDLQVGQVELGGGPGAGSLQPLALEGS | gb\|AAA41439.1\| |
| | LQ | gb\|AAA41442.1\| |
| | E ED QV Q + ELGGGP AG LQ | |
| insulin 1 | LALE +Q | emb\|CAA24559.1\| |
| precursor | (SEQ. ID. No. 17) | gb\|EDL94407.1\| |
| | EVEDPQVPQLELGGGPEAGDLQTLALEVA | GENE ID: 24505 |
| | RQ | Ins1 |
| | Identities = 21/31 (67%), | |
| | Positives = 23/31 | |
| | (74%), Gaps = 0/31 (0%) | |
| Felis catus | (SEQ. ID. No. 1) | ref\|NP_0010092 |
| (Domestic cat) | EAEDLQVGQVELGGGPGAGSLQPLALEGS | 72.1\| |
| | LQ | sp\|P06306.2\|INS_ |
| | EAEDLQ ELG PGAG LQP ALE | FELCA |
| | LQ | dbj\|BAB84110.1\| |
| | (SEQ. ID. No. 18) | GENE ID: |
| | EAEDLQGKDAELGEAPGAGGLQPSALEAP | 493804 INS |
| | LQ | |
| | Identities = 21/31 (67%), | |
| | Positives = 21/31 | |
| | (67%), Gaps = 0/31 (0%) | |
| Golden | (SEQ. ID. No. 1) | sp\|P01313.2\|INS_ |
| hamster | AEDLQVGQVELGGGPGAGSLQPLALE | CRILO |
| | ED QV Q + ELGGGPGA LQ | pir\|\|I48166 |
| | LALE | gb\|AAA37089.1\| |
| | (SEQ. ID. No. 19) | |
| | VEDPQVAQLELGGGPGADDLQTLALE | |
| | Identities = 19/26 (73%), | |
| | Positives = 20/26 | |
| | (76%), Gaps = 0/26 (0%) | |
| Niviventer | (SEQ. ID. No. 1) | gb\|ABB89746.1\| |
| coxingi | EAEDLQVGQVELGGGPGAGSLQPLALEGS | |
| (Coxing's | LQ | |
| white-bellied | E ED QV Q + ELG GP AG LQ | |
| rat) | LALE +Q | |
| | (SEQ. ID. No. 20) | |
| | EVEDPQVAQLELGEGPEAGDLQTLALEVA | |
| | RQ | |
| | Identities = 20/31 (64%), | |
| | Positives = 22/31 | |
| | (70%), Gaps = 0/31 (0%) | |
| Apodemus | (SEQ. ID. No. 1) | gbIABB89744.11 |
| semotus | EAEDLQVGQVELGGGPGAGSLQPLALEGS | |
| (Taiwan field | LQ | |
| mouse) | E ED QV Q + ELGG PG G L + | |
| | LALE +Q | |
| | (SEQ. ID. No. 21) | |
| | EVEDPQVEQLELGGAPGTGDLETLALEVA | |
| | RQ | |
| | Identities = 19/31 (61%), | |
| | Positives = 22/31 | |
| | (70%), Gaps = 0/31 (0%) | |
| Rattus losea | (SEQ. ID. No. 1) | gb\|ABB89743.1\| |
| | EAEDLQVGQVELGGGPGAGSLQPLALEGS | |
| | LQ | |
| | E ED QV Q + ELGG P AG LQ | |
| | LALE + Q | |
| | (SEQ. ID. No. 22) | |
| | EVEDPQVPQLELGGSPEAGDLQTLALEVA | |
| | RQ | |
| | Identities = 20/31 (64%), | |
| | Positives = 22/31 | |
| | (70%), Gaps = 0/31 (0%) | |
| Meriones | (SEQ. ID. No. 1) | gb\|ABB89751.1\| |
| unguiculatus | AEDLQVGQVELGGGPGAGSLQPLALEGSL | |
| (Mongolian | Q | |
| gerbil) | ED Q + Q + ELGG PGAG LQ | |
| | LALE + Q | |
| | (SEQ. ID. No. 23) | |
| | VEDPQMPQLELGGSPGAGDLQALALEVAR | |
| | Q | |
| | Identities = 19/30 (63%), | |
| | Positives = 22/30 | |
| | (73%), Gaps = 0/30 (0%) | |
| Psammomys | (SEQ. ID. No. 1) | sp\|Q62587.1\|INS_ |
| obesus | AEDLQVGQVELGGGPGAGSLQPLALEGSL | PSAOB |
| (Fat sand rat) | Q | emb\|CAA66897.1\| |
| | + D Q + Q + ELGG PGAG L + | |
| | LALE + Q | |
| | (SEQ. ID. No. 24) | |
| | VDDPQMPQLELGGSPGAGDLRALALEVAR | |
| | Q | |
| | Identities = 17/30 (56%), | |
| | Positives = 22/30 | |
| | (73%), Gaps = 0/30 (0%) | |
| Sus scrofa | (SEQ. ID. No. 1) | ref\|NP_0011032 |
| (Pig) | EAEDLQVGQVELGGGPGAGSLQPLALEG | 42.1\| |
| | EAE + Q G VELGG G G LQ | |
| | LALEG | |
| | (SEQ. ID. No. 25) EAENPQAGAVELGG-- | |
| | GLGGLQALALEG | |
| | Identities = 19/28 (67%), | |
| | Positives = 20/28 | |
| | (71%), Gaps = 2/28 (7%) | |
| Rhinolophus | (SEQ. ID. No. 26) | gb\|ACC68945.1\| |
| ferrumequinum | EVEDPQAGQVELGGGPGTGGLQSLALEG | |
| | PPQ | |
| Equus | (SEQ. ID. No. 27) | GENE ID: |
| przewalskii | EAEDPQVGEVELGGGPGLGGLQPLALAGP | 100060077 |
| (Horse) | QQ | LOC100060077 |
| | | gb\|AAB25818.1\| |
| Bos Taurus | (SEQ. ID. No. 28) | gb\|AAI42035.1\| |
| (Bovine) | EVEGPQVGALELAGGPGAGGLEGPPQ | |
| Otolemur | (SEQ. ID. No. 29) | gb\|ACH53103.1\| |
| garnettii | DTEDPQVGQVGLGGSPITGDLQSLALDVP | |
| (Small-eared | PQ | |
| galago) | | |

Thus all such homologues, orthologs, and naturally-occurring isoforms of C-peptide from human as well as other species (SEQ. ID Nos. 1-29) are included in any of the methods and pharmaceutical compositions of the invention, as long as they retain detectable C-peptide activity.

The C-peptides may be in their native form, i.e., as different variants as they appear in nature in different species which may be viewed as functionally equivalent variants of human C-peptide, or they may be functionally equivalent natural derivatives thereof, which may differ in their amino acid sequence, e.g., by truncation (e.g., from the N- or C-terminus or both) or other amino acid deletions, additions, insertions, substitutions, or post-translational modifications. Naturally-occurring chemical derivatives, including post-translational modifications and degradation products of C-peptide, are also specifically included in any of the methods and pharmaceutical compositions of the invention including, e.g., pyroglutamyl, iso-aspartyl, proteolytic, phosphorylated, glycosylated, oxidatized, isomerized, and deaminated variants of C-peptide.

It is known in the art to synthetically modify the sequences of proteins or peptides, while retaining their useful activity, and this may be achieved using techniques which are standard in the art and widely described in the literature, e.g., random or site-directed mutagenesis, cleavage, and ligation of nucleic acids, or via the chemical synthesis or modification of amino acids or polypeptide chains. Similarly it is within the skill in the art to address and/or mitigate immunogenicity concerns if they arise using C-peptide variants, e.g., by the use of automated computer recognition programs to identify potential T cell epitopes, and directed evolution approaches to identify less immunogenic forms.

Any such modifications, or combinations thereof, may be made and used in any of the methods and pharmaceutical compositions of the invention, as long as activity is retained. The C-terminal end of the molecule is known to be important for activity. Preferably, therefore, the C-terminal end of the C-peptide should be preserved in any such C-peptide variants or derivatives, more preferably the C-terminal pentapeptide of C-peptide (EGSLQ) (SEQ. ID. No. 31) should be preserved or sufficient (see Henriksson M et al.: Cell Mol. Life Sci. 62: 1772-1778, (2005)). As mentioned above, modification of an amino acid sequence may be by amino acid substitution, e.g., an amino acid may be replaced by another that preserves the physicochemical character of the peptide (e.g., A may be replaced by G or vice versa, V by A or L; E by D or vice versa; and Q by N). Generally, the substituting amino acid has similar properties, e.g., hydrophobicity, hydrophilicity, electronegativity, bulky side chains, etc., to the amino acid being replaced.

Modifications to the mid-part of the C-peptide sequence (e.g., to residues 13 to 25 of human C-peptide) allow the production of functional derivatives or variants of C-peptide. Thus, C-peptides which may be used in any of the methods or pharmaceutical compositions of the invention may have amino acid sequences which are substantially homologous, or substantially similar to the native C-peptide amino acid sequences, e.g., to the human C-peptide sequence of SEQ. ID. No. 1 or any of the other native C-peptide sequences shown in Table 2. Alternatively, the C-peptide may have an amino acid sequence having at least 30% preferably at least 40, 50, 60, 70, 75, 80, 85, 90, 95, 98, or 99% identity with the amino acid sequence of any one of SEQ. ID. Nos. 1-29 as shown in Table D3, preferably with the native human sequence of SEQ. ID. No. 1. In a preferred embodiment, the C-peptide for use in any of the methods or pharmaceutical compositions of the present invention is at least 80% identical to a sequence selected from Table 2. In another aspect, the C-peptide for use in any of the methods or pharmaceutical compositions of the invention is at least 80% identical to human C-peptide (SEQ. ID. No. 1). Although any amino acid of C-peptide may be altered as described above, it is preferred that one or more of the glutamic acid residues at positions 3, 11, and 27 of human C-peptide (SEQ. ID. No. 1) or corresponding or equivalent positions in C-peptide of other species, are conserved. Preferably, all of the glutamic acid residues at positions 3, 11, and 27 (or corresponding Glu residues) of SEQ. ID. No. 1 are conserved. Alternatively, it is preferred that Glu27 of human C-peptide (or a corresponding Glu residue of a non-human C-peptide) is conserved. An exemplary functional equivalent form of C-peptide which may be used in any of the methods or pharmaceutical compositions of the invention includes the amino acid sequences:

(SEQ. ID. No. 30)
EXEXXQXXXXELXXXXXXXXXXXXALBXXXQ.
(SEQ. ID. No. 33)
GXEXXQXXXXELXXXXXXXXXXXXALBXXXQ.

As used herein, X is any amino acid. The N-terminal residue may be either Glu or Gly (SEQ. ID. No. 30 or SEQ. ID. No. 33, respectively). Functionally equivalent derivatives or variants of native C-peptide sequences may readily be prepared according to techniques well-known in the art, and include peptide sequences having a functional, e.g., a biological activity of a native C-peptide.

Fragments of native or synthetic C-peptide sequences may also have the desirable functional properties of the peptide from which they were derived and may be used in any of the methods or pharmaceutical compositions of the invention. The term “fragment” as used herein thus includes fragments of a C-peptide provided that the fragment retains the biological or therapeutically beneficial activity of the whole molecule. The fragment may also include a C-terminal fragment of C-peptide. Preferred fragments comprise residues 15-31 of native C-peptide, more especially residues 20-31. Peptides comprising the pentapeptide EGSLQ (SEQ. ID. No. 31) (residues 27-31 of native human C-peptide) are also preferred. The fragment may thus vary in size from, e.g., 4 to 30 amino acids or 5 to 20 residues. Suitable fragments are disclosed in WO 98/13384 the contents of which are incorporated herein by reference.

The fragment may also include an N-terminal fragment of C-peptide, typically having the sequence EAEDLQVGQVEL (SEQ. ID. No. 32), or a fragment thereof which comprises 2 acidic amino acid residues, capable of adopting a conformation where said two acidic amino acid residues are spatially separated by a distance of 9-14 A between the alpha-carbons thereof. Also included are fragments having N- and/or C-terminal extensions or flanking sequences. The length of such extended peptides may vary, but typically are not more than 50, 30, 25, or 20 amino acids in length. Representative suitable fragments are described in U.S. Pat. No. 6,610,649, which is hereby incorporated by reference in its entirety.

In such a case it will be appreciated that the extension or flanking sequence will be a sequence of amino acids which is not native to a naturally-occurring or native C-peptide, and in particular a C-peptide from which the fragment is derived. Such a N- and/or C-terminal extension or flanking sequence may comprise, e.g., from 1 to 10, 1 to 6, 1 to 5, 1 to 4, or 1 to 3 amino acids.

The term “derivative” as used herein thus refers to C-peptide sequences or fragments thereof, which have modifications as compared to the native sequence. Such modifications may be one or more amino acid deletions, additions, insertions, and/or substitutions. These may be contiguous or non-contiguous. Representative variants may include those having 1 to 6, or more preferably 1 to 4, 1 to 3, or 1 or 2 amino acid substitutions, insertions, and/or deletions as compared to any of SEQ. ID. Nos. 1-33. The substituted amino acid may be any amino acid, particularly one of the well-known 20 conventional amino acids (Ala (A); Cys (C); Asp (D); Glu (E); Phe (F); Gly (G); His (H); Ile (I); Lys (K); Leu (L); Met (M); Asn (N); Pro (P); Gin (Q); Arg (R); Ser (5); Thr (T); Val (V); Trp (W); and Tyr (Y)). Any such variant or derivative of C-peptide may be used in any of the methods or pharmaceutical compositions of the invention.

Isomers of the native L-amino acids, e.g., D-amino acids may be incorporated in any of the above forms of C-peptide, and used in any of the methods or pharmaceutical compositions of the invention. Additional variants may include amino and/or carboxyl terminal fusions as well as intrasequence insertions of single or multiple amino acids. Longer peptides may comprise multiple copies of one or more of the C-peptide sequences, such as any of SEQ. ID. Nos. 1-33. Insertional amino acid sequence variants are those in which one or more amino acid residues are introduced at a site in the protein. Deletional variants are characterized by the removal of one or more amino acids from the sequence. Variants may include, e.g., different allelic variants as they appear in nature, e.g., in other species or due to geographical variation. All such variants, derivatives, fusion proteins, or fragments of C-peptide are included, may be used in any of the methods claims or pharmaceutical compositions disclosed herein, and are subsumed under the term “C-peptide”.

The conjugated forms of C-peptide, C-peptide variants, derivatives, and fragments thereof are functionally equivalent in that they have detectable C-peptide activity. More particularly, they exhibit at least about 1%, at least about 5%, at least about 10%, at least about 15%, at least about 20%, at least about 30%, at least about 40%, at least about 50%, at least about 60%, at least about 70%, at least about 80%, at least about 90%, at least about 100%, or higher than 100% of the activity of native proinsulin C-peptide, particularly human C-peptide. Thus, they are capable of functioning as proinsulin C-peptide, i.e., can substitute for C-peptide itself. Such activity means any activity exhibited by a native C-peptide, whether a physiological response exhibited in an in vivo or in vitro test system, or any biological activity or reaction mediated by a native C-peptide, e.g., in an enzyme assay or in binding to test tissues, membranes, or metal ions. Thus, it is known that C-peptide causes an influx of calcium and initiates a range of intracellular signalling cascades such as phosphorylation of the MAP-kinase pathway including phosphorylation of ERK 1 and 2, CREB, PKC, GSK3, PI3K, NF-kappaB, and PPARgamma, resulting in an increased expression of eNOS, Na+K+ATPase and a wide range of transcription factors. An assay for C-peptide activity can thus be made by assaying for the activation or up-regulation of any of these pathways upon addition or administration of the peptide (e.g., fragment or derivative) in question to cells from relevant target tissues including endothelial, kidney, fibroblast and immune cells. Such assays are described in, e.g., Ohtomo Y et al. (Diabetologia 39: 199-205, (1996)), Kunt T et al. (Diabetologia 42(4): 465-471, (1999)), Shafqat J et al. (Cell Mol. Life Sci. 59: 1185-1189, (2002)). Kitamura T et al. (Biochem. J. 355: 123-129, (2001)), Hills and Brunskill (Exp Diab Res 2008), as described in WO 98/13384 or in Ohtomo Y et al. (supra) or Ohtomo Y et al. (Diabetologia 41: 287-291, (1998)). An assay for C-peptide activity based on endothelial nitric oxide synthase (eNOS) activity is also described in Kunt T et al. (supra) using bovine aortic cells and a reporter cell assay. Binding to particular cells may also be used to assess or assay for C-peptide activity, e.g., to cell membranes from human renal tubular cells, skin fibroblasts, and saphenous vein endothelial cells using fluorescence correlation spectroscopy, as described, e.g., in Rigler R et al. (PNAS USA 96: 13318-13323, (1999)), Henriksson M et al. (Cell Mol. Life Sci. 57: 337-342, (2000)) and Pramanik A et al. (Biochem Biophys. Res. Commun. 284: 94-98, (2001)).

In another aspect of any of the claimed conjugated C-peptides, the conjugated C-peptide has a plasma or sera pharmacokinetic AUC profile at least about 5-fold greater than unmodified C-peptide when subcutaneously administered to a mammal.

In another aspect of any of the claimed conjugated C-peptides, the conjugated C-peptide has a plasma or sera pharmacokinetic AUC profile at least about 6-fold greater than unmodified C-peptide when subcutaneously administered to a mammal.

In another aspect of any of the claimed conjugated C-peptides, the conjugated C-peptide has a plasma or sera pharmacokinetic AUC profile at least about 7-fold greater than unmodified C-peptide when subcutaneously administered to a mammal.

In another aspect of any of the claimed conjugated C-peptides, the conjugated C-peptide has a plasma or sera pharmacokinetic AUC profile at least about 8-fold greater than unmodified C-peptide when subcutaneously administered to a mammal.

In another aspect of any of the claimed conjugated C-peptides, the conjugated C-peptide has a plasma or sera pharmacokinetic AUC profile at least about 10-fold greater than unmodified C-peptide when subcutaneously administered to a mammal.

In another aspect of any of the claimed conjugated C-peptides, the conjugated C-peptide has a plasma or sera pharmacokinetic AUC profile at least about 15-fold greater than unmodified C-peptide when subcutaneously administered to a mammal.

In another aspect of any of the claimed conjugated C-peptides, the conjugated C-peptide has a plasma or sera pharmacokinetic AUC profile at least about 20-fold greater than unmodified C-peptide when subcutaneously administered to a mammal.

In another aspect of any of the claimed conjugated C-peptides, the conjugated C-peptide has a plasma or sera pharmacokinetic AUC profile at least about 25-fold greater than unmodified C-peptide when subcutaneously administered to a mammal.

In another aspect of any of the claimed conjugated C-peptides, the conjugated C-peptide has a plasma or sera pharmacokinetic AUC profile at least about 50-fold greater than unmodified C-peptide when subcutaneously administered to a mammal.

In another aspect of any of the claimed conjugated C-peptides, the conjugated C-peptide has a plasma or sera pharmacokinetic AUC profile at least about 75-fold greater than unmodified C-peptide when subcutaneously administered to a mammal.

In another aspect of any of the claimed conjugated C-peptides, the conjugated C-peptide has a plasma or sera pharmacokinetic AUC profile at least about 100-fold greater than unmodified C-peptide when subcutaneously administered to a mammal.

In one aspect the mammal is a dog. In one aspect the mammal is a rat. In one aspect the mammal is a human.

### VIII. C-Peptide and Conjugated C-Peptide Production

C-peptide may be produced synthetically using standard solid-phase peptide synthesis, or by recombinant technology, e.g., as a by-product in the production of human insulin from human proinsulin, or using genetically modified host (see generally WO 1999007735; Jonasson P, et al., J Biotechnol. (2000) 76(2-3):215-26; Jonasson P, et al., Gene (1998);210(2):203-10; Li S X, Tian et al., Sheng Wu Hua Xue Yu Sheng Wu Wu Li Xue Bao (Shanghai) (2003) 35(11):986-92; Nilsson J, et al., J Biotechnol. (1996) 48(3):241-50; Huang Y B, et al., Acta Biochim Biophys Sin (Shanghai) (2006) 38(8):586-92).

In an alternative approach to direct coupling to the N-terminus, the modification reagent, or a lysine residue, may be incorporated at a desired position of the C-peptide during peptide synthesis. In this way, site-selective introduction of one or more PEGs can be achieved. See, e.g., International Patent Publication No. WO 95/00162, which describes the site selective synthesis of conjugated peptides.

C-peptide can be produced by expressing a DNA sequence encoding the C-peptide in question in a suitable host cell by well known techniques used for insulin biosynthesis as disclosed in, e.g., U.S. Pat. No. 6,500,645. The C-peptide may be expressed directly, or as a multimerized construct to increase the yield of product as disclosed in U.S. Pat. No. 6,558,924. The multimerized product is cleaved in vitro after isolation from the culture broth.

The polynucleotide sequence coding for the C-peptide may be prepared synthetically by established standard methods, e.g., the phosphoamidite method described by Beaucage et al. (1981) Tetrahedron Letters 22:1859-1869, or the method described by Matthes et al. (1984) EMBO Journal 3:801-805. According to the phosphoramidite method, oligonucleotides are synthesized, e.g., in an automatic DNA synthesizer, purified, duplexed and ligated to form the synthetic DNA construct. A currently preferred way of preparing the DNA construct is by polymerase chain reaction (PCR).

The polynucleotide sequences may also be of mixed genomic, cDNA, and synthetic origin. For example, a genomic or cDNA sequence encoding a leader peptide may be joined to a genomic or cDNA sequence encoding the A and B chains, after which the DNA sequence may be modified at a site by inserting synthetic oligonucleotides encoding the desired amino acid sequence for homologous recombination in accordance with well-known procedures or preferably generating the desired sequence by PCR using suitable oligonucleotides.

The recombinant method will typically make use of a vector which is capable of replicating in the selected microorganism or host cell and which carries a polynucleotide sequence encoding the parent single-chain insulin of the invention. The recombinant vector may be an autonomously replicating vector, i.e., a vector which exists as an extra-chromosomal entity, the replication of which is independent of chromosomal replication, e.g., a plasmid, an extra-chromosomal element, a mini-chromosome, or an artificial chromosome.

The vector may contain any means for assuring self-replication. Alternatively, the vector may be one which, when introduced into the host cell, is integrated into the genome and replicated together with the chromosome(s) into which it has been integrated. Furthermore, a single vector or plasmid or two or more vectors or plasmids which together contain the total DNA to be introduced into the genome of the host cell, or a transposon may be used. The vector may be linear or closed circular plasmids and will preferably contain an element(s) that permits stable integration of the vector into the host cell's genome or autonomous replication of the vector in the cell independent of the genome.

The recombinant expression vector is capable of replicating in yeast. Examples of sequences which enable the vector to replicate in yeast are the yeast plasmid 2 pm replication genes REP 1-3 and origin of replication. The vector may contain one or more selectable markers which permit easy selection of transformed cells. A selectable marker is a gene the product of which provides for biocide or viral resistance, resistance to heavy metals, prototroph to auxotroph, and the like. Examples of bacterial selectable markers are the dal genes from Bacillus subtilis or Bacillus licheniformis, or markers which confer antibiotic resistance such as ampicillin, kanamycin, chloramphenicol or tetracycline resistance. Selectable markers for use in a filamentous fungal host cell include amdS (acetamidase), argB (ornithine carbamoyltransferase), pyrG (orotidine-5′-phosphate decarboxylase) and trpC (anthranilate synthase. Suitable markers for yeast host cells are ADE2, H153, LEU2, LYS2, MET3, TRP1, and URA3. A well-suited selectable marker for yeast is the Schizosaccharomyces pompe TPI gene (Russell (1985) Gene 40:125-130).

In the vector, the polynucleotide sequence is operably connected to a suitable promoter sequence. The promoter may be any nucleic acid sequence which shows transcriptional activity in the host cell of choice including mutant, truncated, and hybrid promoters, and may be obtained from genes encoding extra-cellular or intracellular polypeptides either homologous or heterologous to the host cell.

Examples of suitable promoters for directing the transcription in a bacterial host cell are the promoters obtained from the E. coli lac operon, Streptomyces coelicolor agarase gene (dagA), Bacillus subtilis levansucrase gene (sacB), Bacillus licheniformis alpha-amylase gene (amyL), Bacillus stearothermophilus maltogenic amylase gene (amyM), Bacillus amyloliquefaciens alpha-amylase gene (amyQ), and Bacillus licheniformis penicillinase gene (penP). Examples of suitable promoters for directing the transcription in a filamentous fungal host cell are promoters obtained from the genes for Aspergillus oryzae TAKA amylase, Rhizomucor miehei aspartic proteinase, Aspergillus niger neutral alpha-amylase, and Aspergillus niger acid stable alpha-amylase. In a yeast host, useful promoters are the Saccharomyces cerevisiae Mal, TPI, ADH, or PGK promoters. The polynucleotide sequence encoding the C-peptide of the invention will also typically be operably connected to a suitable terminator. In yeast a suitable terminator is the TPI terminator (Alber et al. (1982) J. Mol. Appl. Genet. 1:419-434).

The procedures used to ligate the polynucleotide sequence encoding the parent single-chain insulin of the invention, the promoter and the terminator, respectively, and to insert them into a suitable vector containing the information necessary for replication in the selected host, are well known to persons skilled in the art. It will be understood that the vector may be constructed either by first preparing a DNA construct containing the entire DNA sequence encoding the single-chain insulins of the invention, and subsequently inserting this fragment into a suitable expression vector, or by sequentially inserting DNA fragments encoding genetic information for the individual elements followed by ligation.

The vector comprising the polynucleotide sequence encoding the C-peptide of the invention is introduced into a host cell so that the vector is maintained as a chromosomal integrant or as a self-replicating extra-chromosomal vector. The term “host cell” encompasses any progeny of a parent cell that is not identical to the parent cell due to mutations that occur during replication. The host cell may be a unicellular microorganism, e.g., a prokaryote, or a non-unicellular microorganism, e.g., a eukaryote. Useful unicellular cells are bacterial cells such as gram positive bacteria including, but not limited to, a Bacillus cell, Streptomyces cell, or gram negative bacteria such as E. coli and Pseudomonas sp. Eukaryote cells may be mammalian, insect, plant, or fungal cells. In one embodiment, the host cell is a yeast cell. The yeast organism may be any suitable yeast organism which, on cultivation, produces large amounts of the single chain insulin of the invention. Examples of suitable yeast organisms are strains selected from the yeast species Saccharomyces cerevisiae, Saccharomyces kluyveri, Schizosaccharomyces pombe, Sacchoromyces uvarum, Kluyvero-myces lactis, Hansenula polymorpha, Pichia pastoris, Pichia methanolica, Pichia kluyveri, Yarrowia ilpolytica, Candida sp., Candida utilis, Candida cacaoi, Geotrichum sp., and Geotrichum fermentans.

The transformation of the yeast cells may for instance be effected by protoplast formation followed by transformation in a manner known per se. The medium used to cultivate the cells may be any conventional medium suitable for growing yeast organisms. The secreted single-chain insulin, a significant proportion of which will be present in the medium in correctly processed form, may be recovered from the medium by conventional procedures including separating the yeast cells from the medium by centrifugation, filtration or catching the insulin precursor by an ion exchange matrix or by a reverse phase absorption matrix, precipitating the proteinaceous components of the supernatant or filtrate by means of a salt, e.g., ammonium sulphate, followed by purification by a variety of chromatographic procedures, e.g., ion exchange chromatography, affinity chromatography, or the like.

### IX. Methods of Use

In one aspect, the present invention includes a method for maintaining C-peptide levels above the minimum effective therapeutic level in a patient in need thereof, comprising administering to the patient a therapeutic dose of any of the claimed conjugated C-peptides.

In another aspect, the present invention includes a method for treating one or more long-term complications of diabetes in a patient in need thereof, comprising administering to the patient a therapeutic dose of any of the claimed conjugated C-peptides.

In another aspect, the present invention includes a method for treating a patient with diabetes comprising administering to the patient a therapeutic dose of any of the claimed conjugated C-peptides in combination with insulin.

In another aspect, the present invention includes any of the claimed conjugated C-peptides for use as a C-peptide replacement therapy or dose in a patient in need thereof.

In broad terms, diabetes refers to the situation where the body either fails to properly respond to its own insulin, does not make enough insulin, or both. The primary result of impaired insulin production is the accumulation of glucose in the blood, and a C-peptide deficiency leading to various short- and long-term complications. Three principal forms of diabetes exist:

Type 1: Results from the body's failure to produce insulin and C-peptide. It is estimated that 5-10% of Americans who are diagnosed with diabetes have type 1 diabetes. Presently almost all persons with type 1 diabetes must take insulin injections. The term “type 1 diabetes” has replaced several former terms, including childhood-onset diabetes, juvenile diabetes, and insulin-dependent diabetes mellitus (IDDM). For patients with type 1 diabetes, basal levels of C-peptide are typically less than about 0.20 nM (Ludvigsson et al.: New Engl. J. Med. 359: 1909-1920, (2008)).

Type 2: Results from tissue insulin resistance, a condition in which cells fail to respond properly to insulin, sometimes combined with relative insulin deficiency. The term “type 2 diabetes” has replaced several former terms, including adult-onset diabetes, obesity-related diabetes, and non-insulin-dependent diabetes mellitus (NIDDM). For type 2 patients in the basal state, C-peptide levels of about 0.8 nM (range 0.64 to 1.56 nM), and glucose stimulated levels of about 5.7 nM (range 3.7 to 7.7 nM) have been reported. (Retnakaran R et al.: Diabetes Obes. Metab. (2009) DOI 10.11 111/j.1463-1326.2009.01129.x; Zander et al.: Lancet 359: 824-830, (2002)).

In addition to type 1 and type 2 diabetics, there is increasing recognition of a subclass of diabetes referred to as latent autoimmune diabetes in the adult (LADA) or late-onset autoimmune diabetes of adulthood, or “slow onset type 1” diabetes, and sometimes also “type 1.5” or “type one-and-a-half” diabetes. In this disorder, diabetes onset generally occurs in ages 35 and older, and antibodies against components of the insulin-producing cells are always present, demonstrating that autoimmune activity is an important feature of LADA. It is primarily antibodies against glutamic acid decarboxylase (GAD) that are found. Some LADA patients show a phenotype similar to that of type 2 patients with increased body mass index (BMI) or obesity, insulin resistance, and abnormal blood lipids. Genetic features of LADA are similar to those for both type 1 and type 2 diabetes. During the first 6-12 months after debut the patients may not require insulin administration and they are able to maintain relative normoglycemia via dietary modification and/or oral anti-diabetic medication. However, eventually all patients become insulin dependent, probably as a consequence of progressive autoimmune activity leading to gradual destruction of the pancreatic islet β-cells. At this stage the LADA patients show low or absent levels of endogenous insulin and C-peptide, and they are prone to develop long-term complications of diabetes involving the peripheral nerves, the kidneys, or the eyes similar to type 1 diabetes patients and thus become candidates for C-peptide therapy (Palmer et al.: Diabetes 54(suppl 2): S62-67, (2005); Desai et al.: Diabetic Medicine 25(suppl 2): 30-34, (2008); Fourlanos et al.: Diabetologia 48: 2206-2212, (2005)).

Gestational diabetes: Pregnant women who have never had diabetes before but who have high blood sugar (glucose) levels during pregnancy are said to have gestational diabetes. Gestational diabetes affects about 4% of all pregnant women. It may precede development of type 2 (or rarely type 1) diabetes.

Several other forms of diabetes mellitus are categorized separately from these. Examples include congenital diabetes due to genetic defects of insulin secretion, cystic fibrosis-related diabetes, steroid diabetes induced by high doses of glucocorticoids, and several forms of monogenic diabetes.

Accordingly in any of these methods, the term “patient” refers to an individual who has one of more of the symptoms of any of diabetes. In one aspect of any of these methods, the term “patient” refers to an individual who has one of more of the symptoms of any of insulin-dependent diabetes. In one aspect of any of these methods, the term “patient” refers to an individual who has one of more of the symptoms of any of type 2 diabetes. In one aspect of any of these methods, the term “patient” refers to an individual who has one of more of the symptoms of LADA. In one aspect of any of these methods, the term “patient” refers to an individual who has one of more of the symptoms of gestational diabetes. Accordingly in one aspect of any of these methods, the term “patient” refers to an individual who has a fasting C-peptide level of less than about 0.4 nM. In another aspect of any of these methods, the term “patient” refers to an individual who has a fasting C-peptide level of less than about 0.2 nM.

Acute complications of diabetes include hypoglycemia, diabetic ketoacidosis, or nonketotic hyperosmolar coma that may occur if the disease is not adequately controlled. Serious long-term complications can also occur, and are discussed in more detail below.

In another aspect, the present invention includes a method for treating one or more long-term complications of diabetes in a patient in need thereof, comprising administering to the patient a therapeutic dose of any of the claimed conjugated C-peptides.

In another aspect, the present invention includes a method for treating a patient with diabetes comprising administering to the patient a therapeutic dose of any of the claimed conjugated C-peptides in combination with insulin.

In this context “in combination” means: 1) part of the same unitary dosage form; 2) administration separately, but as part of the same therapeutic treatment program or regimen, typically but not necessarily, on the same day. In one aspect, any of the claimed conjugated C-peptides may be administered at a fixed daily dosage, and the insulin taken on an as needed basis.

In another aspect, the present invention includes any of the claimed conjugated C-peptides for use for treating one or more long-term complications of diabetes in a patient in need thereof.

In any of these methods, the terms “long-term complication of type 1 diabetes”, or “long-term complications of diabetes” refers to the long-term complications of impaired glycemic control, and C-peptide deficiency associated with insulin-dependent diabetes. Typically long-term complications of type 1 diabetes are associated with type 1 diabetics. However the term can also refer to long-term complications of diabetes that arise in type 1.5 and type 2 diabetic patients who develop a C-peptide deficiency as a consequence of losing pancreatic islet β-cells and therefore also become insulin dependent. In broad terms, many such complications arise from the primary damage of blood vessels (angiopathy), resulting in subsequent problems that can be grouped under “microvascular disease” (due to damage to small blood vessels) and “macrovascular disease” (due to damage to the arteries).

Specific diseases and disorders included within the term long-term complications of diabetes include, without limitation; retinopathy including early stage retinopathy with microaneurysms, proliferative retinopathy, and macular edema; peripheral neuropathy including sensorimotor polyneuropathy, painful sensory neuropathy, acute motor neuropathy, cranial focal and multifocal polyneuropathies, thoracolumbar radiculoneuropathies, proximal diabetic neuropathies, and focal limb neuropathies including entrapment and compression neuropathies; autonomic neuropathy involving the cardiovascular system, the gastrointestinal tract, the respiratory system, the urigenital system, sudomotor function and papillary function; and nephropathy including disorders with microalbuminuria, overt proteinuria, and end-stage renal disease.

Impaired microcirculatory perfusion appears to be crucial to the pathogenesis of both neuropathy and retinopathy in diabetics. This in turn reflects a hyperglycemia-mediated perturbation of vascular endothelial function that results in: over-activation of protein kinase C, reduced availability of nitric oxide (NO), increased production of superoxide and endothelin-1 (ET-1), impaired insulin function, diminished synthesis of prostacyclin/PGE1, and increased activation and endothelial adherence of leukocytes. This is ultimately a catastrophic group of clinical events.

Accordingly in some embodiments, the term “patient” refers to an individual who has one of more of the symptoms of the long-term complications of diabetes.

Diabetic retinopathy is an ocular manifestation of the systemic damage to small blood vessels leading to microangiopathy. In retinopathy, growth of friable and poor-quality new blood vessels in the retina as well as macular edema (swelling of the macula) can lead to severe vision loss or blindness. As new blood vessels form at the back of the eye as a part of proliferative diabetic retinopathy (PDR), they can bleed (hemorrhage) and blur vision. It affects up to 80% of all patients who have had diabetes for 10 years or more.

The symptoms of diabetic retinopathy are often slow to develop and subtle and include blurred version and progressive loss of sight. Macular edema, which may cause vision loss more rapidly, may not have any warning signs for some time. In general, however, a person with macular edema is likely to have blurred vision, making it hard to do things like read or drive. In some cases, the vision will get better or worse during the day.

Accordingly in some embodiments, the term “patient” refers to an individual who has one of more of the symptoms of diabetic retinopathy.

Diabetic neuropathies are neuropathic disorders that are associated with diabetic microvascular injury involving small blood vessels that supply nerves (vasa nervorum). Relatively common conditions which may be associated with diabetic neuropathy include third nerve palsy; mononeuropathy; mononeuropathy multiplex; diabetic amyotrophy; a painful polyneuropathy; autonomic neuropathy; and thoracoabdominal neuropathy.

Diabetic neuropathy affects all peripheral nerves: pain fibers, motor neurons, autonomic nerves. It therefore necessarily can affect all organs and systems since all are innervated. There are several distinct syndromes based on the organ systems and members affected, but these are by no means exclusive. A patient can have sensorimotor and autonomic neuropathy or any other combination. Symptoms vary depending on the nerve(s) affected and may include symptoms other than those listed. Symptoms usually develop gradually over years.

Symptoms of diabetic neuropathy may include: numbness and tingling of extremities, dysesthesia (decreased or loss of sensation to a body part), diarrhea, erectile dysfunction, urinary incontinence (loss of bladder control), impotence, facial, mouth and eyelid drooping, vision changes, dizziness, muscle weakness, difficulty swallowing, speech impairment, fasciculation (muscle contractions), anorgasmia, and burning or electric pain.

Additionally, different nerves are affected in different ways by neuropathy. Sensorimotor polyneuropathy, in which longer nerve fibers are affected to a greater degree than shorter ones, because nerve conduction velocity is slowed in proportion to a nerve's length. In this syndrome, decreased sensation and loss of reflexes occurs first in the toes on each foot, then extends upward. It is usually described as glove-stocking distribution of numbness, sensory loss, dysesthesia, and night-time pain. The pain can feel like burning, pricking sensation, achy, or dull. Pins and needles sensation is common. Loss of proprioception, the sense of where a limb is in space, is affected early. These patients cannot feel when they are stepping on a foreign body, like a splinter, or when they are developing a callous from an ill-fitting shoe. Consequently, they are at risk for developing ulcers and infections on the feet and legs, which can lead to amputation. Similarly, these patients can get multiple fractures of the knee, ankle, or foot, and develop a Charcot joint. Loss of motor function results in dorsiflexion, contractures of the toes, loss of the interosseous muscle function, and leads to contraction of the digits, so called hammer toes. These contractures occur not only in the foot, but also in the hand where the loss of the musculature makes the hand appear gaunt and skeletal. The loss of muscular function is progressive.

Autonomic neuropathy impacts the autonomic nervous system serving the heart, gastrointestinal system, and genitourinary system. The most commonly recognized autonomic dysfunction in diabetics is orthostatic hypotension, or fainting when standing up. In the case of diabetic autonomic neuropathy, it is due to the failure of the heart and arteries to appropriately adjust heart rate and vascular tone to keep blood continually and fully flowing to the brain. This symptom is usually accompanied by a loss of the usual change in heart rate seen with normal breathing. These two findings suggest autonomic neuropathy.

Gastrointestinal system symptoms include delayed gastric emptying, gastroparesis, nausea, bloating, and diarrhea. Because many diabetics take oral medication for their diabetes, absorption of these medicines is greatly affected by the delayed gastric emptying. This can lead to hypoglycemia when an oral diabetic agent is taken before a meal and does not get absorbed until hours, or sometimes days later, when there is normal or low blood sugar already. Sluggish movement of the small intestine can cause bacterial overgrowth, made worse by the presence of hyperglycemia. This leads to bloating, gas, and diarrhea.

Genitourinary system symptoms associated with autonomic neuropathy include urinary frequency, urgency, incontinence, retention, impotence, and erectile dysfunction. Urinary retention can lead to bladder diverticula, stones, reflux nephropathy, and frequent urinary tract infections. Administration of C-peptide has been shown to improve erectile function in insulin-requiring diabetic patients (Wahren et al.: Diabetes 60, Suppl 1: A285, (2011)). Accordingly in any of these methods, the term “patient” refers to an individual who has one of more of the symptoms of autonomic neuropathy. In certain methods, the term “patient” refers to an individual who has one or more symptoms of erectile dysfunction or impotence.

Accordingly in some embodiments, the term “patient” refers to an individual who has one of more of the symptoms of diabetic neuropathy. In another aspect of any of these methods, the patient has “established peripheral neuropathy” which is characterized by reduced sensory nerve conduction velocity (SCV) in the sural nerves (less than −1.5 SD from a body height-corrected reference value for a matched normal individual). In certain embodiments, the term “patient” refers to an individual who has one of more of the symptoms of incipient neuropathy.

Accordingly in certain embodiments, the current invention includes a method of treating or preventing a decrease in a subject's, or patient's, height-adjusted sensory or motor nerve conduction velocity. In one aspect of this method, the motor nerve conduction velocity is initial nerve conduction velocity. In another embodiment, the motor nerve conduction velocity is the peak nerve conduction velocity.

In certain embodiments the subject is a patient with diabetes. In certain embodiments, the subject has at least one long term complication of diabetes. In one aspect, the patient exhibits a peak nerve conduction velocity that is at least about 2 standard deviations from the mean peak nerve conduction velocity for a similar height-matched subject group. In one aspect, the patients have a peak nerve conduction velocity of greater than about 35 m/s. In one aspect of any of the claimed methods, the patients have a peak nerve conduction velocity of greater than about 40 m/s. In one aspect, the patients have a peak nerve conduction velocity of greater than about 45 m/s. In one aspect, the patients have a peak nerve conduction velocity of greater than about 50 m/s.

In one aspect of any of the claimed methods, treatment results in an improvement in nerve conduction velocity of at least about 1.5 m/s. In another aspect of these methods, treatment results in an improvement in nerve conduction velocity of at least about 2.0 m/s. In another aspect of these methods, treatment results in an improvement in nerve conduction velocity of at least about 2.5 m/s. In another aspect of these methods, treatment results in an improvement in nerve conduction velocity of at least about 3.0 m/s. In another aspect of these methods, treatment results in an improvement in nerve conduction velocity of at least about 3.5 m/s. In another aspect of these methods, treatment results in an improvement in nerve conduction velocity of at least about 4.0 m/s. In another aspect of these methods, treatment results in an improvement in nerve conduction velocity of at least about 4.5 m/s. In another aspect of these methods, treatment results in an improvement in nerve conduction velocity of at least about 5.0 m/s. In another aspect of these methods, treatment results in an improvement in nerve conduction velocity of at least about 5.5 m/s. In another aspect of these methods, treatment results in an improvement in nerve conduction velocity of at least about 6.0 m/s. In another aspect of these methods, treatment results in an improvement in nerve conduction velocity of at least about 7.0 m/s. In another aspect of these methods, treatment results in an improvement in nerve conduction velocity of at least about 8.0 m/s. In another aspect of these methods, treatment results in an improvement in nerve conduction velocity of at least about 9.0 m/s. In another aspect of these methods, treatment results in an improvement in nerve conduction velocity of at least about 10.0 m/s. In another aspect of these methods, treatment results in an improvement in nerve conduction velocity of at least about 15.0 m/s. In another aspect of these methods, treatment results in an improvement in nerve conduction velocity of at least about 20.0 m/s.

In certain embodiments, of any of these methods, treatment results in an improvement of at least 10% in peak nerve conduction velocity compared to peak nerve conduction velocity prior to starting conjugated C-peptide therapy. In certain embodiments, of any of these methods, treatment results in an improvement of at least 15% in peak nerve conduction velocity compared to peak nerve conduction velocity prior to starting conjugated C-peptide therapy. In certain embodiments, of any of these methods, treatment results in an improvement of at least 20% in peak nerve conduction velocity compared to peak nerve conduction velocity prior to starting conjugated C-peptide therapy. In certain embodiments, of any of these methods, treatment results in an improvement of at least 25% in peak nerve conduction velocity compared to peak nerve conduction velocity prior to starting conjugated C-peptide therapy. In certain embodiments, of any of these methods, treatment results in an improvement of at least 30% in peak nerve conduction velocity compared to peak nerve conduction velocity prior to starting conjugated C-peptide therapy. In certain embodiments, of any of these methods, treatment results in an improvement of at least 40% in peak nerve conduction velocity compared to peak nerve conduction velocity prior to starting conjugated C-peptide therapy. In certain embodiments, of any of these methods, treatment results in an improvement of at least 50% in peak nerve conduction velocity compared to peak nerve conduction velocity prior to starting conjugated C-peptide therapy.

Diabetic nephropathy is a progressive kidney disease caused by angiopathy of capillaries in the kidney glomeruli. It is characterized by nephrotic syndrome and diffuse glomerulosclerosis. It is due to long-standing diabetes mellitus, and is a prime cause for dialysis in many Western countries.

The symptoms of diabetic nephropathy can be seen in patients with chronic diabetes (15 years or more after onset). The disease is progressive and is more frequent in men. Diabetic nephropathy is the most common cause of chronic kidney failure and end-stage kidney disease in the United States. People with both type 1 and type 2 diabetes are at risk. The risk is higher if blood-glucose levels are poorly controlled. Further, once nephropathy develops, the greatest rate of progression is seen in patients with poor control of their blood pressure. Also people with high cholesterol level in their blood have much more risk than others.

The earliest detectable change in the course of diabetic nephropathy is an abnormality of the glomerular filtration barrier. At this stage, the kidney may start allowing more serum albumin than normal in the urine (albuminuria), and this can be detected by sensitive medical tests for albumin. This stage is called “microalbuminuria”. As diabetic nephropathy progresses, increasing numbers of glomeruli are destroyed by nodular glomerulosclerosis. Now the amounts of albumin being excreted in the urine increases, and may be detected by ordinary urinalysis techniques. At this stage, a kidney biopsy clearly shows diabetic nephropathy.

Kidney failure provoked by glomerulosclerosis leads to fluid filtration deficits and other disorders of kidney function. There is an increase in blood pressure (hypertension) and fluid retention in the body plus a reduced plasma oncotic pressure causes edema. Other complications may be arteriosclerosis of the renal artery and proteinuria.

Throughout its early course, diabetic nephropathy has no symptoms. They develop in late stages and may be a result of excretion of high amounts of protein in the urine or due to renal failure. Symptoms include, edema; swelling, usually around the eyes in the mornings; later, general body swelling may result, such as swelling of the legs, foamy appearance or excessive frothing of the urine (caused by the proteinura), unintentional weight gain (from fluid accumulation), anorexia (poor appetite), nausea and vomiting, malaise (general ill feeling), fatigue, headache, frequent hiccups, and generalized itching.

Accordingly in some embodiments, the term “patient” refers to an individual who has one of more of the symptoms of diabetic nephropathy.

Diabetic cardiomyopathy (DCM), damage to the heart, leading to diastolic dysfunction and eventually heart failure. Aside from large vessel disease and accelerated atherosclerosis, which is very common in diabetes, DCM is a clinical condition diagnosed when ventricular dysfunction develops in patients with diabetes in the absence of coronary atherosclerosis and hypertension. DCM may be characterized functionally by ventricular dilation, myocyte hypertrophy, prominent interstitial fibrosis, and decreased or preserved systolic function in the presence of a diastolic dysfunction.

One particularity of DCM is the long latent phase, during which the disease progresses but is completely asymptomatic. In most cases, DCM is detected with concomitant hypertension or coronary artery disease. One of the earliest signs is mild left ventricular diastolic dysfunction with little effect on ventricular filling. Also, the diabetic patient may show subtle signs of DCM related to decreased left ventricular compliance or left ventricular hypertrophy or a combination of both. A prominent “a” wave can also be noted in the jugular venous pulse, and the cardiac apical impulse may be overactive or sustained throughout systole. After the development of systolic dysfunction, left ventricular dilation and symptomatic heart failure, the jugular venous pressure may become elevated and the apical impulse would be displaced downward and to the left. Systolic mitral murmur is not uncommon in these cases. These changes are accompanied by a variety of electrocardiographic changes that may be associated with DCM in 60% of patients without structural heart disease, although usually not in the early asymptomatic phase. Later in the progression, a prolonged QT interval may be indicative of fibrosis. Given that the definition of DCM excludes concomitant atherosclerosis or hypertension, there are no changes in perfusion or in atrial natriuretic peptide levels up until the very late stages of the disease, when the hypertrophy and fibrosis become very pronounced.

In certain embodiments, the term “patient” refers to an individual who has one of more of the symptoms of diabetic cardiomyopathy.

Macrovascular diseases of diabetes include coronary artery disease, leading to angina or myocardial infarction (“heart attack”), stroke (mainly the ischemic type), peripheral vascular disease, which contributes to intermittent claudication (exertion-related leg and foot pain), as well as diabetic foot and diabetic myonecrosis (“muscle wasting”).

In certain embodiments, the term “patient” refers to an individual who has one of more of the symptoms of a macrovascular disease of diabetes.

### Methods for Preventing Hypoglycemia.

In certain embodiments, the present invention includes the use of any of the disclosed conjugated C-peptides to reduce the risk of hypoglycemia in a human patient with insulin dependent diabetes, in a regimen which additionally comprises the administration of insulin, comprising; a) administering insulin to said patient; b) administering a therapeutic dose of conjugated C-peptide in a different site as that used for said patient's insulin administration; c) adjusting the dosage amount, type, or frequency of insulin administered based on said patient's altered insulin requirements resulting from said therapeutic dose of conjugated C-peptide.

In another aspect, the present invention includes a method of reducing insulin usage in an insulin-dependent human patient, comprising the steps of; a) administering insulin to said patient; b) administering subcutaneously to said patient a therapeutic dose of any of the disclosed conjugated C-peptides in a different site as that used for said patient's insulin administration; c) adjusting the dosage amount, type, or frequency of insulin administered based on monitoring said patient's altered insulin requirements resulting from said therapeutic dose of conjugated C-peptide, wherein said adjusted dose of insulin does not induce hyperglycemia, wherein said adjusted dose of insulin is at least 10% less than said patient's insulin dose prior to starting conjugated C-peptide. (See for example U.S. Pat. No. 7,855,177, which is herein incorporated by reference).

In any of these methods, the term “hypoglycemia” or “hypoglycemic events” refers to all episodes of abnormally low plasma glucose concentration that exposes the patient to potential harm. The American Diabetes Association Workgroup has recommended that people with insulin-dependent diabetes become concerned about the possibility of developing hypoglycemia at a plasma glucose concentration of less than 70 mg/dL (3.9 mmoL/L). Accordingly in one aspect of any of the claimed methods, the terms hypoglycemia or hypoglycemic event refers to the situation where the plasma glucose concentration of the patient drops to less than about 70 mg/dL (3.9 mmoL/L).

Hypoglycemia is a serious medical complication in the treatment of diabetes, and causes recurrent morbidity in most people with type 1 diabetes and many with advanced type 2 diabetes and is sometimes fatal. In addition, hypoglycemia compromises physiological and behavioral defenses against subsequent falling plasma glucose concentrations and thus causes a vicious cycle of recurrent hypoglycemia. Accordingly the prevention of hypoglycemia is of significant importance in the treatment of diabetes, as well as the treatment of the long-term complications of diabetes.

Unfortunately hypoglycemia is a fact of life for most people with type 1 diabetes (Cryer P E et al.: Diabetes 57: 3169-3176, (2008)). The average patient has untold numbers of episodes of asymptomatic hypoglycemia and suffers two episodes of symptomatic hypoglycemia per week, with thousands of such episodes over a lifetime of diabetes. He or she suffers one or more episodes of severe, temporarily disabling hypoglycemia often with seizure or coma, per year.

Overall, hypoglycemia is less frequent in type 2 diabetes; however, the risk of hypoglycemia becomes progressively more frequent and limiting to glycemic control later in the course of type 2 diabetes. The prospective, population-based data of Donnelly et al. (Diabetes Med. 22: 749-755, (2005)) indicate that the overall incidence of hypoglycemia in insulin-treated type 2 diabetes is approximately one third of that in type 1 diabetes. The incidence of any hypoglycemia and of severe hypoglycemia was 4,300 and 115 episodes per 100 patient years, respectively, in type 1 diabetes and 1600 and 35 episodes per 100 patient years, respectively, in insulin-treated type 2 diabetes.

Hypoglycemia may be classified based on the severity of the hypoglycemic event. For example, the American Diabetes Association Workgroup has suggested the following classification of hypoglycemia in diabetes: 1) severe hypoglycemia (i.e., hypoglycemic coma requiring assistance of another person); 2) documented symptomatic hypoglycemia (with symptoms and a plasma glucose concentration of less than 70 mg/dL); 3) asymptomatic hypoglycemia (with a plasma glucose concentration of less than 70 mg/dL without symptoms); 4) probable symptomatic hypoglycemia (with symptoms attributed to hypoglycemia, but without a plasma glucose measurement); and 5) relative hypoglycemia (with a plasma glucose concentration of greater than 70 mg/dL but falling towards that level).

Thus in another aspect of any of the methods disclosed herein, the term “hypoglycemia” refers to severe hypoglycemia, and/or hypoglycemic coma. In another aspect of any of these methods, the term “hypoglycemia” refers to symptomatic hypoglycemia. In another aspect of any of these methods, the term “hypoglycemia” refers to probable symptomatic hypoglycemia. In another aspect of any of these methods, the term “hypoglycemia” refers to asymptomatic hypoglycemia. In another aspect of any of these methods, the term “hypoglycemia” refers to relative hypoglycemia.

### Insulin Types and Administration Forms

There are over 180 individual insulin preparations available worldwide which have been developed to provide different lengths of activity (activity profiles). Approximately 25% of these are soluble insulin (unmodified form); about 35% are long- or intermediate-acting basal insulins (mixed with NPH [neutral protamine Hagedorn] insulin or Lente insulin [insulin zinc suspension], or forms that are modified to have an increased isoelectric point [insulin glargine], or acylation [insulin detemir]; these forms have reduced solubility, slow subcutaneous absorption, and long duration of action relative to soluble insulins); about 2% are rapid-acting insulins (e.g., which are engineered by amino acid change, and have reduced self-association and increased subcutaneous absorption); and about 38% are pre-mixed insulins (e.g., mixtures of short-, intermediate-, and long-acting insulins; these preparations have the benefit of a reduced number of daily injections).

Short-acting insulin preparations that are commercially available in the US include regular insulin and rapid-acting insulins. Regular insulin has an onset of action of 30-60 minutes, peak time of effect of 1.5 to 2 hours, and duration of activity of 5 to 12 hours. Rapid-acting insulins, such as Aspart (Novo Rapid), Lispro (Humalog), and Glulisine (Apidra), have an onset of action of 10-30 minutes, peak time of effect of around 30 minutes, and a duration of activity of 3 to 5 hours.

Intermediate-acting insulins, such as NPH and Lente insulins, have an onset of action of 1 to 2 hours, peak time of effect of 4 to 8 hours, and a duration of activity of 10 to 20 hours.

Long-acting insulins, such as Ultralente insulin, have an onset of action of 2 to 4 hours, peak time of effect of 8 to 20 hours, and a duration of activity of 16 to 24 hours. Other examples of long-acting insulins include Glargine and Determir. Glargine insulin has an onset of action of 1 to 2 hours, and a duration of action of 24 hours, but with no peak effect.

In many cases, regimens that use insulin in the management of diabetes combine long-acting and short-acting insulin. For example, Lantus, from Aventis Pharmaceuticals Inc., is a recombinant human insulin analog that is a long-acting, parenteral blood-glucose-lowering agent whose longer duration of action (up to 24 hours) is directly related to its slower rate of absorption. Lantus is administered subcutaneously once a day, preferably at bedtime, and is said to provide a continuous level of insulin, similar to the slow, steady (basal) secretion of insulin provided by the normal pancreas. The activity of such a long-acting insulin results in a relatively constant concentration/time profile over 24 hours with no pronounced peak, thus allowing it to be administered once a day as a patient's basal insulin. Such long-acting insulin has a long-acting effect by virtue of its chemical composition, rather than by virtue of an addition to insulin when administered.

More recently automated wireless controlled systems for continuous infusion of insulin, such as the system sold under the trademark OMNIPOD™ Insulin Management System (Insulet Corporation, Bedford, Mass.) have been developed. These systems provide continuous subcutaneous insulin delivery with blood glucose monitoring technology in a discreet two-part system. This system eliminates the need for daily insulin injections, and does not require a conventional insulin pump which is connected via tubing.

OMNIPOD™ is a small lightweight device that is worn on the skin like an infusion set. It delivers insulin according to pre-programmed instructions transmitted wirelessly from the Personal Diabetes Manager (PDM). The PDM is a wireless, hand-held device that is used to program the OMNIPOD™ Insulin Management System with customized insulin delivery instructions, monitor the operation of the system, and check blood glucose levels using blood glucose test strips sold under the trademark FREESTYLE™. There is no tubing connecting the device to the PDM. OMNIPOD™ Insulin Management System is worn beneath the clothing, and the PDM can be carried separately in a backpack, briefcase, or purse. Similar to currently available insulin pumps, the OMNIPOD™ Insulin Management System features fully programmable continuous subcutaneous insulin delivery with multiple basal rates and bolus options, suggested bolus calculations, safety checks, and alarm features.

The aim of insulin treatment of diabetics is typically to administer enough insulin such that the patient will have blood glucose levels within the physiological range and normal carbohydrate metabolism throughout the day. Because the pancreas of a diabetic individual does not secrete sufficient insulin throughout the day, in order to effectively control diabetes through insulin therapy, a long-lasting insulin treatment, known as basal insulin, must be administered to provide the slow and steady release of insulin that is needed to control blood glucose concentrations and to keep cells supplied with energy when no food is being digested. Basal insulin is necessary to suppress glucose production between meals and overnight and preferably mimics the patient's normal pancreatic basal insulin secretion over a 24-hour period. Thus, a diabetic patient may administer a single dose of a long-acting insulin each day subcutaneously, with an action lasting about 24 hours.

Furthermore, in order to effectively control diabetes through insulin therapy by dealing with postprandial rises in glucose levels, a bolus, fast-acting treatment must also be administered. The bolus insulin, which is generally administered subcutaneously, provides a rise in plasma insulin levels at approximately 1 hour after administration, thereby limiting hyperglycemia after meals. Thus, these additional quantities of regular insulin, with a duration of action of, e.g., 5 to 6 hours, may be subcutaneously administered at those times of the day when the patient's blood glucose level tends to rise too high, such as at meal times. As an alternative to administering basal insulin in combination with bolus insulin, repeated and regular lower doses of bolus insulin may be administered in place of the long-acting basal insulin, and bolus insulin may be administered postprandially as needed.

Currently, regular subcutaneously injected insulin is recommended to be dosed at 30 to 45 minutes prior to mealtime. As a result, diabetic patients and other insulin users must engage in considerable planning of their meals and of their insulin administrations relative to their meals. Unfortunately, intervening events that may take place between administration of insulin and ingestion of the meal may affect the anticipated glucose excursions.

Furthermore, there is also the potential for hypoglycemia if the administered insulin provides a therapeutic effect over too great a time, e.g., after the rise in glucose levels that occur as a result of ingestion of the meal has already been lowered. As outlined in the Examples, this risk of hypoglycemia is increased in patients who have been treated with C-peptide due to a reduced requirement for insulin.

Accordingly, in one aspect of any of the methods disclosed herein, the present invention includes a method for reducing the risk of the patient developing hypoglycemia by reducing the average daily dose of insulin administered to the patient by about 5% to about 50% after starting conjugated C-peptide therapy. In another aspect, the dose of insulin administered is reduced by about 5% to about 45% compared to the patient's insulin dose prior to starting conjugated C-peptide treatment. In another aspect, the dose of insulin administered is reduced by about 5% to about 40% compared to the patient's insulin dose prior to starting conjugated C-peptide treatment. In another aspect, the dose of insulin administered is reduced by about 5% to about 35% compared to the patient's insulin dose prior to starting conjugated C-peptide treatment. In another aspect, the dose of insulin administered is reduced by about 5% to about 30% compared to the patient's insulin dose prior to starting conjugated C-peptide treatment. In another aspect, the dose of insulin administered is reduced by about 5% to about 25% compared to the patient's insulin dose prior to starting conjugated C-peptide treatment. In another aspect, the dose of insulin administered is reduced by about 5% to about 20% compared to the patient's insulin dose prior to starting conjugated C-peptide treatment. In another aspect, the dose of insulin administered is reduced by about 5% to about 15% compared to the patient's insulin dose prior to starting conjugated C-peptide treatment. In another aspect, the dose of insulin administered is reduced by about 5% to about 10% compared to the patient's insulin dose prior to starting conjugated C-peptide treatment.

In another aspect, the dose of insulin administered is reduced by about 2% to about 10% compared to the patient's insulin dose prior to starting conjugated C-peptide treatment. In another aspect, the dose of insulin administered is reduced by about 2% to about 15% compared to the patient's insulin dose prior to starting conjugated C-peptide treatment. In another aspect, the dose of insulin administered is reduced by about 2% to about 20% compared to the patient's insulin dose prior to starting conjugated C-peptide treatment.

In another aspect, the dose of insulin administered is reduced by about 10% to about 50% compared to the patient's insulin dose prior to starting conjugated C-peptide treatment. In another aspect, the dose of insulin administered is reduced by about 10% to about 45% compared to the patient's insulin dose prior to starting conjugated C-peptide treatment. In another aspect, the dose of insulin administered is reduced by about 10% to about 40% compared to the patient's insulin dose prior to starting conjugated C-peptide treatment. In another aspect, the dose of insulin administered is reduced by about 10% to about 35% compared to the patient's insulin dose prior to starting C-peptide treatment. In another aspect, the dose of insulin administered is reduced by about 10% to about 30% compared to the patient's insulin dose prior to starting conjugated C-peptide treatment. In another aspect, the dose of insulin administered is reduced by about 10% to about 25% compared to the patient's insulin dose prior to starting conjugated C-peptide treatment. In another aspect, the dose of insulin administered is reduced by about 10% to about 20% compared to the patient's insulin dose prior to starting conjugated C-peptide treatment. In another aspect, the dose of insulin administered is reduced by at least 10% compared to the patient's insulin dose prior to starting conjugated C-peptide treatment.

In one aspect of any of these methods, the dose of short-acting insulin administered is selectively reduced by any of the prescribed ranges listed above. In another aspect of any of these methods, the dose of intermediate-acting insulin administered is selectively reduced by any of the prescribed ranges. In one aspect of any of these methods, the dose of long-acting insulin administered is selectively reduced by any of the prescribed ranges listed above.

In another aspect of any of these methods, the dose of intermediate- and long-acting insulin administered is independently reduced by any of the prescribed ranges listed above, while the dose of short-acting insulin remains substantially unchanged.

In one aspect of these methods, the dose of short-acting insulin administered is reduced by about 5% to about 50% compared to the patient's insulin dose prior to starting conjugated C-peptide treatment. In another embodiment, the dose of short-acting insulin administered is reduced by about 5% to about 35% compared to the patient's insulin dose prior to starting conjugated C-peptide treatment. In another embodiment, the dose of short-acting insulin administered is reduced by about 10% to about 20% compared to the patient's insulin dose prior to starting conjugated C-peptide treatment. In one aspect of these methods, the dose of short-acting insulin administered preprandially for a meal is reduced. In another aspect of these methods, the dose of short-acting insulin administered in the morning or at nighttime is reduced. In another aspect of any of these methods, the dose of short-acting insulin administered is reduced while the dose of long-acting and/or intermediate-acting insulin administered to the patient is substantially unchanged.

In another aspect of any of the methods disclosed herein, the present invention includes a method for reducing the risk of the patient developing hypoglycemia by reducing the average daily dose of intermediate-acting insulin administered to the patient by about 5% to about 35% after starting conjugated C-peptide therapy. In one aspect of these methods, the dose of intermediate-acting insulin administered is reduced by about 5% to about 50% compared to the patient's insulin dose prior to starting conjugated C-peptide treatment. In another embodiment, the dose of intermediate-acting insulin administration is reduced by about 5% to about 35% compared to the patient's insulin dose prior to starting conjugated C-peptide treatment. In another embodiment, the dose of intermediate-acting insulin administered is reduced by about 10% to about 20% compared to the patient's insulin dose prior to starting conjugated C-peptide treatment. In another aspect of these methods, the dose of intermediate-acting insulin administered in the morning or at nighttime is reduced. In another aspect of any of these methods, the dose of intermediate-acting insulin administered is reduced while the dose of short-acting insulin administered to the patient is substantially unchanged.

In another aspect of any of the methods disclosed herein, the present invention includes a method for reducing the risk of the patient developing hypoglycemia by reducing the average daily dose of long-acting insulin administered to the patient by about 5% to about 50% after starting conjugated C-peptide therapy. In one embodiment, the dose of long-acting insulin administered is reduced by about 5% to about 35% compared to the patient's insulin dose prior to starting conjugated C-peptide treatment. In another embodiment, the dose of long-acting insulin administered is reduced by about 10% to about 20% compared to the patient's insulin dose prior to starting conjugated C-peptide treatment. In another aspect of these methods, the dose of long-acting insulin administered in the morning or at nighttime is reduced. In another aspect of any of these methods, the dose of long-acting insulin administered is reduced while the dose of short-acting insulin administered to the patient is substantially unchanged.

In certain preferred embodiments, the patient achieves improved insulin utilization and insulin sensitivity while experiencing a reduced risk of developing hypoglycemia after treatment with conjugated C-peptide as compared with baseline levels prior to treatment. Preferably, the improved insulin utilization and insulin sensitivity are measured by a statistically significant decline in HOMA (Homeostasis Model Assessment) (Turner et al.: Metabolism 28(11): 1086-1096, (1979)).

Subcutaneous administration of the conjugated C-peptide will typically not be into the same site as that most recently used for insulin administration, i.e. conjugated C-peptide and insulin will be injected into different sites. Specifically in one aspect, the site of PEGylated administration will typically be at least about 10 cm way from the site most recently used for insulin administration. In another aspect, the site of conjugated C-peptide administration will typically be at least about 15 cm away from the site most recently used for insulin administration. In another aspect, the site of conjugated C-peptide administration will typically be at least about 20 cm away from the site most recently used for insulin administration.

Examples of different sites include for example, and without limitation, injections into the left and right arm, or injections into the left and right thigh, or injections into the left or right buttock, or injections into the opposite sides of the abdomen. Other obvious variants of different sites include injections in an arm and thigh, or injections in an arm and buttock, or injections into an arm and abdomen, etc.

Moreover one of ordinary skill in the art, i.e. a physician, or diabetic patient, will recognize and understand how to inject conjugated C-peptide and insulin into any other combination of different sites, based on the prior art teaching, and numerous text books and guides on insulin administration that provide disclosure on how to select different insulin injection sites. See for example, the following representative text books (Learning to live well with diabetes, Ed. Cheryl Weiler, (1991) DCI Publishing, Minneapolis, Minn.; American Diabetes Association Complete Guide to Diabetes, ISBN 0-945448-64-3 (1996)).

In one aspect of any of the claimed methods, conjugated C-peptide is administered to the opposite side of the abdomen to the site most recently used for insulin administration, approximately 15 to 20 cm apart.

### X. Pharmaceutical Compositions

In one aspect, the present invention includes a pharmaceutical composition comprising conjugated C-peptide, and a pharmaceutically acceptable carrier, diluent or excipient.

Pharmaceutical compositions suitable for the delivery of conjugated C-peptide and methods for their preparation will be readily apparent to those skilled in the art and may comprise any of the known carriers, diluents, or excipients. Such compositions and methods for their preparation may be found, e.g., in Remington's Pharmaceutical Sciences, 19th Edition (Mack Publishing Company, 1995).

In one aspect, the pharmaceutical compositions may be in the form of sterile aqueous solutions and/or suspensions of the pharmaceutically active ingredients, aerosols, ointments, and the like. Formulations which are aqueous solutions are most preferred. Such formulations typically contain the conjugated C-peptide itself, water, and one or more buffers which act as stabilizers (e.g., phosphate-containing buffers) and optionally one or more preservatives. Such formulations containing, e.g., about 1 to 200 mg, about 3 to 100 mg, about 3 to 80 mg, about 3 to 60 mg, about 3 to 40 mg, about 3 to 30 mg, about 0.3 to 3.3 mg, about 1 to 3.3 mg, about 1 to 2 mg, about 1 to 3.3 mg, about 2 to 3.3 mg or any of the ranges mentioned herein, e.g., about 200 mg, about 150 mg, about 120 mg, about 100 mg, about 80 mg, about 60 mg, about 50 mg, about 40 mg, about 30 mg, about 20 mg, or about 10 mg, or about 8 mg, or about 6 mg, or about 5 mg, or about 4 mg, or about 3 mg, or about 2 mg, or about 1 mg, or about 0.5 mg of the conjugated C-peptide and constitute a further aspect of the invention.

Pharmaceutical compositions may include pharmaceutically acceptable salts of conjugated C-peptide. For a review on suitable salts, see Handbook of Pharmaceutical Salts: Properties, Selection, and Use by Stahl and Wermuth (Wiley-VCH, 2002). Suitable base salts are formed from bases which form non-toxic salts. Representative examples include the aluminium, arginine, benzathine, calcium, choline, diethylamine, diolamine, glycine, lysine, magnesium, meglumine, olamine, potassium, sodium, tromethamine, and zinc salts. Hemisalts of acids and bases may also be formed, e.g., hemisulphate and hemicalcium salts. In one embodiment, conjugated C-peptide may be prepared as a gel with a pharmaceutically acceptable positively charged ion.

In one aspect, the positively charged ion may be a monovalent metal ion. In one aspect, the metal ion is selected from sodium and potassium.

In one aspect, the positively charged ion may be a divalent metal ion. In one aspect, the metal ion is selected from calcium, magnesium, and zinc.

The conjugated C-peptide may be administered at any time during the day. For humans, the dosage used may range from about 0.1 to 200 mg/week of conjugated C-peptide, e.g., from about 0.1 to 0.3 mg/week, about 0.3 to 1.5 mg/week, about 1 mg to about 3.5 mg/week, about 1.5 to 2.25 mg/week, about 2.25 to 3.0 mg/week, about 3.0 to 6.0 mg/week, about 6.0 to 10 mg/week, about 10 to 20 mg/week, about 20 to 40 mg/week, about 40 to 60 mg/week, about 60 to 80 mg/week, about 80 to 100 mg/week, about 100 to 120 mg/week, about 120 to 140 mg/week, about 140 to 160 mg/week, about 160 to 180 mg/week, and about 180 to about 200 mg/week.

Preferably the total weekly dose used of conjugated C-peptide is about 1 mg to about 3.5 mg, about 1 mg to about 20 mg, about 20 mg to about 50 mg, about 50 mg to about 100 mg, about 100 mg to about 150 mg, or about 150 mg to about 200 mg.

The total weekly dose of conjugated C-peptide may be about 0.1 mg, about 0.5 mg, about 1 mg, about 1.5 mg, about 2 mg, about 2.5 mg, about 3 mg, about 3.5 mg, about 4 mg, about 4.5 mg, about 5 mg, about 5.5 mg, about 6 mg, about 7 mg, about 8 mg, about 9 mg, about 10 mg, about 12 mg, about 15 mg, about 18 mg, about 21 mg, about 24 mg, about 27 mg, about 30 mg, about 33 mg, about 36 mg, about 39 mg, about 42 mg, about 45 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, or about 200 mg. (It will be appreciated that masses of conjugated C-peptide referred to above are dependent on the bioavailability of the delivery system and based on the use of conjugated C-peptide with a molecular mass of approximately 40,000 Da.)

In another aspect of any of these methods and pharmaceutical compositions, the therapeutic dose of conjugated C-peptide comprises a weekly dose ranging from about 1 mg to about 45 mg. In another aspect of any of these methods and pharmaceutical compositions, the therapeutic dose of conjugated C-peptide comprises a weekly dose ranging from about 3 mg to about 15 mg. In another aspect of any of these methods and pharmaceutical compositions, the therapeutic dose of conjugated C-peptide comprises a weekly dose ranging from about 30 mg to about 60 mg. In another aspect of any of these methods and pharmaceutical compositions, the therapeutic dose of conjugated C-peptide comprises a weekly dose ranging from about 60 mg to about 120 mg.

In another aspect of any of these methods and pharmaceutical compositions, the therapeutic dose of conjugated C-peptide maintains the average steady-state concentration of conjugated C-peptide (Cₛₛ₋ₐᵥₑ) in the patient's plasma of between about 0.2 nM and about 6 nM.

In another aspect of any of these methods and pharmaceutical compositions, the therapeutic dose of conjugated C-peptide is provided to the patient so as to maintain the average steady-state concentration of conjugated C-peptide in the patient's plasma between about 0.2 nM and about 6 nM when using a dosing interval of 3 days or longer. In another aspect of any of these methods and pharmaceutical compositions, the therapeutic dose of conjugated C-peptide is provided to the patient so as to maintain the average steady-state concentration of conjugated C-peptide in the patient's plasma between about 0.2 nM and about 6 nM when using a dosing interval of 4 days or longer. In another aspect of any of these methods and pharmaceutical compositions, the therapeutic dose of conjugated C-peptide is provided to the patient so as to maintain the average steady-state concentration of conjugated C-peptide in the patient's plasma between about 0.2 nM and about 6 nM when using a dosing interval of 5 days or longer. In another aspect of any of these methods and pharmaceutical compositions, the therapeutic dose of conjugated C-peptide is provided to the patient so as to maintain the average steady-state concentration of conjugated C-peptide in the patient's plasma between about 0.2 nM and about 6 nM when using a dosing interval of at least one week. In any of these methods and pharmaceutical compositions, the therapeutic dose is administered by daily subcutaneous injections. In another aspect of any of these methods and pharmaceutical compositions, the therapeutic dose is administered by a sustained release formulation or device.

In another aspect of any of these methods and pharmaceutical compositions, the therapeutic dose of conjugated C-peptide is provided to the patient so as to maintain the average steady-state concentration of conjugated C-peptide in the patient's plasma between about 0.4 nM and about 8 nM when using a dosing interval of 3 days or longer. In another aspect of any of these methods and pharmaceutical compositions, the therapeutic dose of conjugated C-peptide is provided to the patient so as to maintain the average steady-state concentration of conjugated C-peptide in the patient's plasma between about 0.4 nM and about 8 nM when using a dosing interval of 4 days or longer. In another aspect of any of these methods and pharmaceutical compositions, the therapeutic dose of conjugated C-peptide is provided to the patient so as to maintain the average steady-state concentration of conjugated C-peptide in the patient's plasma between about 0.4 nM and about 8 nM when using a dosing interval of 5 days or longer. In another aspect of any of these methods and pharmaceutical compositions, the therapeutic dose of conjugated C-peptide is provided to the patient so as to maintain the average steady-state concentration of conjugated C-peptide in the patient's plasma between about 0.4 nM and about 8 nM when using a dosing interval of 7 days or longer.

In another aspect of any of these methods and pharmaceutical compositions, the therapeutic dose of conjugated C-peptide is provided to the patient so as to maintain the average steady-state concentration of conjugated C-peptide in the patient's plasma between about 0.6 nM and about 8 nM when using a dosing interval of 3 days or longer. In another aspect of any of these methods and pharmaceutical compositions, the therapeutic dose of conjugated C-peptide is provided to the patient so as to maintain the average steady-state concentration of conjugated C-peptide in the patient's plasma between about 0.6 nM and about 8 nM when using a dosing interval of 4 days or longer. In another aspect of any of these methods and pharmaceutical compositions, the therapeutic dose of conjugated C-peptide is provided to the patient so as to maintain the average steady-state concentration of conjugated C-peptide in the patient's plasma between about 0.6 nM and about 8 nM when using a dosing interval of 5 days or longer. In another aspect of any of these methods and pharmaceutical compositions, the therapeutic dose of conjugated C-peptide is provided to the patient so as to maintain the average steady-state concentration of conjugated C-peptide in the patient's plasma between about 0.6 nM and about 8 nM when using a dosing interval of 7 days or longer.

The dose may or may not be in solution. If the dose is administered in solution, it will be appreciated that the volume of the dose may vary, but will typically be 20 μL-2 mL. Preferably the dose for S.C. administration will be given in a volume of 2000 μL, 1500 μL, 1200 μL, 1000 μL, 900 μL, 800 μL, 700 μL, 600 μL, 500 μL, 400 μL, 300 μL, 200 μL, 100 μL, 50 μL, or 20 μL.

PEGylated C-peptide doses in solution can also comprise a preservative and/or a buffer. For example, the preservatives m-cresol, or phenol can be used. Typical concentrations of preservatives include 0.5 mg/mL, 1 mg/mL, 2 mg/mL, 3 mg/mL, 4 mg/mL, or 5 mg/mL. Thus, a range of concentration of preservative may include 0.2 to 10 mg/mL, particularly 0.5 to 6 mg/mL, or 0.5 to 5 mg/mL. Examples of buffers that can be used include histidine (pH 6.0), sodium phosphate buffer (pH 6 to 7.5), or sodium bicarbonate buffer (pH 7 to 7.5). It will be appreciated that the conjugated C-peptide dose may comprise one or more of a native or intact C-peptide, fragments, derivatives, or other functionally equivalent variants of C-peptide.

### XI. Methods for Administration

A dose of conjugated C-peptide may comprise full-length human C-peptide (SEQ. ID. No. 1) and the C-terminal C-peptide fragment EGSLQ (SEQ. ID. No. 31) and/or a C-peptide homolog or C-peptide derivative. Further, the dose may if desired only contain a fragment of C-peptide, e.g., EGSLQ. Thus, the term “C-peptide” may encompass a single C-peptide entity or a mixture of different “C-peptides”. Administration of the conjugated C-peptide may be by any suitable method known in the medicinal arts, including oral, parenteral, topical, or subcutaneous administration, inhalation, or the implantation of a sustained delivery device or composition. In one aspect, administration is by subcutaneous administration.

Pharmaceutical compositions of the invention suitable for oral administration may, e.g., comprise conjugated C-peptide in sterile purified stock powder form preferably covered by an envelope or envelopes (enterocapsules) protecting from degradation of the drug in the stomach and thereby enabling absorption of these substances from the gingiva or in the small intestines. The total amount of active ingredient in the composition may vary from 99.99 to 0.01 percent of weight.

For oral administration a pharmaceutical composition comprising a conjugated C-peptide can take the form of solutions, suspensions, tablets, pills, capsules, powders, and the like. Tablets containing various excipients such as sodium citrate, calcium carbonate and calcium phosphate are employed along with various disintegrants such as starch and preferably potato or tapioca starch and certain complex silicates, together with binding agents such as polyvinylpyrrolidone, sucrose, gelatin and acacia. Additionally, lubricating agents such as magnesium stearate, sodium lauryl sulfate and talc are often very useful for tabletting purposes. Solid compositions of a similar type are also employed as fillers in soft and hard-filled gelatin capsules; preferred materials in this connection also include lactose or milk sugar as well as high molecular weight polyethylene glycols. When aqueous suspensions and/or elixirs are desired for oral administration, the compounds of this invention can be combined with various sweetening agents, flavoring agents, coloring agents, emulsifying agents and/or suspending agents, as well as such diluents as water, ethanol, propylene glycol, glycerin and various like combinations thereof.

Pharmaceutical compositions to be used in the invention suitable for parenteral administration are typically sterile aqueous solutions and/or suspensions of the pharmaceutically active ingredients preferably made isotonic with the blood of the recipient. Such compositions generally comprise excipients, salts, carbohydrates, and buffering agents (preferably to a pH of from 3 to 9), such as sodium chloride, glycerin, glucose, mannitol, sorbitol, and the like.

For some applications, pharmaceutical compositions for parenteral administration may be suitably formulated as a sterile non-aqueous solution or as a dried form to be used in conjunction with a suitable vehicle such as sterile, pyrogen-free water. The preparation of parenteral formulations under sterile conditions, e.g., by lyophilization, may readily be accomplished using standard pharmaceutical techniques well-known to those skilled in the art.

Pharmaceutical compositions comprising conjugated C-peptide for use in the present invention may also be administered topically, (intra)dermally, or transdermally to the skin or mucosa. Pharmaceutical compositions for topical administration may be formulated to be immediate and/or modified release. Modified release formulations include delayed, sustained, pulsed, controlled, targeted and programmed release. Typical formulations for this purpose include gels, hydrogels, lotions, solutions, creams, ointments, dusting powders, dressings, foams, films, skin patches, wafers, implants, sponges, fibers, bandages, and microemulsions. Liposomes may also be used. Typical carriers include alcohol, water, mineral oil, liquid petrolatum, white petrolatum, glycerin, polyethylene glycol, and propylene glycol. Penetration enhancers may be incorporatedsee, e.g., Finnin and Morgan: J. Pharm. ScL 88(10): 955-958, (1999). Other means of topical administration include delivery by electroporation, iontophoresis, phonophoresis, sonophoresis, and microneedle or needle-free (e.g., POWDERJECT™, BOJECT™) injection.

Pharmaceutical compositions of conjugated C-peptide for parenteral administration may be administered directly into the blood stream, into muscle, or into an internal organ. Suitable means for parenteral administration include intravenous, intra-arterial, intraperitoneal, intrathecal, intraventricular, intraurethral, intrasternal, intracranial, intramuscular, intrasynovial, and subcutaneous. Suitable devices for parenteral administration include needle (including microneedle) injectors, needle-free injectors, and infusion techniques.

Subcutaneous administration of conjugated C-peptide will typically not be into the same site as that most recently used for insulin administration. In one aspect of any of the claimed methods and pharmaceutical compositions, conjugated C-peptide is administered to the opposite side of the abdomen to the site most recently used for insulin administration. In another aspect of any of the claimed methods and pharmaceutical compositions, conjugated C-peptide is administered to the upper arm. In another aspect of any of the claimed methods and pharmaceutical compositions, conjugated C-peptide is administered to the abdomen. In another aspect of any of the claimed methods and pharmaceutical compositions, conjugated C-peptide is administered to the upper area of the buttock. In another aspect of any of the claimed methods and pharmaceutical compositions, conjugated C-peptide is administered to the front of the thigh.

Formulations for parenteral administration may be formulated to be immediate and/or sustained release. Sustained release compositions include delayed, modified, pulsed, controlled, targeted and programmed release. Thus conjugated C-peptide may be formulated as a suspension or as a solid, semi-solid, or thixotropic liquid for administration as an implanted depot providing sustained release. Examples of such formulations include without limitation, drug-coated stents and semi-solids and suspensions comprising drug-loaded poly(DL-lactic-co-glycolic)acid (PGLA), poly(DL-lactide-co-glycolide) (PLG) or poly(lactide) (PLA) lamellar vesicles or microparticles, hydrogels (Hoffman A S: Ann. N.Y. Acad. ScL 944: 62-73 (2001)), poly-amino acid nanoparticles systems, such as the Medusa system developed by Flame! Technologies Inc., nonaqueous gel systems such as Atrigel developed by Atrix, Inc., and SABER (Sucrose Acetate Isobutyrate Extended Release) developed by Durect Corporation, and lipid-based systems such as DepoFoam developed by SkyePharma.

Sustained release devices capable of delivering desired doses of conjugated C-peptide over extended periods of time are known in the art. For example, U.S. Pat. Nos. 5,034,229; 5,557,318; 5,110,596; 5,728,396; 5,985,305; 6,113,938; 6,156,331; 6,375,978; and 6,395,292; teach osmotically-driven devices capable of delivering an active agent formulation, such as a solution or a suspension, at a desired rate over an extended period of time (i.e., a period ranging from more than one week up to one year or more). Other exemplary sustained release devices include regulator-type pumps that provide constant flow, adjustable flow, or programmable flow of beneficial agent formulations, which are available from, e.g., OmniPod™ Insulin Management System (Insulet Corporation, Codman of Raynham, Mass., Medtronic of Minneapolis, Minn., Intarcia Therapeutics of Hayward, Calif., and Tricumed Medinzintechnik GmbH of Germany). Further examples of devices are described in U.S. Pat. Nos. 6,283,949; 5,976,109; 5,836,935; and 5,511,355.

Because they can be designed to deliver a desired active agent at therapeutic levels over an extended period of time, implantable delivery systems can advantageously provide long-term therapeutic dosing of a desired active agent without requiring frequent visits to a healthcare provider or repetitive self-medication. Therefore, implantable delivery devices can work to provide increased patient compliance, reduced irritation at the site of administration, fewer occupational hazards for healthcare providers, reduced waste hazards, and increased therapeutic efficacy through enhanced dosing control.

Among other challenges, two problems must be addressed when seeking to deliver biomolecular material over an extended period of time from an implanted delivery device. First, the biomolecular material must be contained within a formulation that substantially maintains the stability of the material at elevated temperatures (i.e., 37° C. and above) over the operational life of the device. Second, the biomolecular material must be formulated in a way that allows delivery of the biomolecular material from an implanted device into a desired environment of operation over an extended period of time. This second challenge has proven particularly difficult where the biomolecular material is included in a flowable composition that is delivered from a device over an extended period of time at low flow rates (i.e., 100 pliday).

Peptide drugs such as C-peptide may degrade via one or more of several different mechanisms, including deamidation, oxidation, hydrolysis, and racemization. Significantly, water is a reactant in many of the relevant degradation pathways. Moreover, water acts as a plasticizer and facilitates the unfolding and irreversible aggregation of biomolecular materials. To work around the stability problems created by aqueous formulations of biomolecular materials, dry powder formulations of biomolecular materials have been created using known particle formation processes, such as by known lyophilization, spray drying, or desiccation techniques. Though dry powder formulations of biomolecular material have been shown to provide suitable stability characteristics, it would be desirable to provide a formulation that is not only stable over extended periods of time, but is also flowable and readily deliverable from an implantable delivery device.

Accordingly in one aspect of any of the claimed methods and pharmaceutical compositions, the conjugated C-peptide is provided in a nonaqueous drug formulation, and is delivered from a sustained release implantable device, wherein the conjugated C-peptide is stable for at least two months of time at 37° C.

Representative nonaqueous formulations for conjugated C-peptide include those disclosed in International Publication Number WO00/45790 that describes nonaqueous vehicle formulations that are formulated using at least two of a polymer, a solvent, and a surfactant.

WO98/27962 discloses an injectable depot gel composition containing a polymer, a solvent that can dissolve the polymer and thereby form a viscous gel, a beneficial agent, and an emulsifying agent in the form of a dispersed droplet phase in the viscous gel.

WO04089335 discloses nonaqueous vehicles that are formed using a combination of polymer and solvent that results in a vehicle that is miscible in water. As it is used herein, the term “miscible in water” refers to a vehicle that, at a temperature range representative of a chosen operational environment, can be mixed with water at all proportions without resulting in a phase separation of the polymer from the solvent such that a highly viscous polymer phase is formed. For the purposes of the present invention, a “highly viscous polymer phase” refers to a polymer containing composition that exhibits a viscosity that is greater than the viscosity of the vehicle before the vehicle is mixed with water.

Accordingly in another aspect of any of the claimed methods, conjugated C-peptide is provided in a sustained release device comprising: a reservoir having at least one drug delivery orifice, and a stable nonaqueous drug formulation. In one aspect of these methods and pharmaceutical compositions, the formulation comprises: at least conjugated C-peptide; and a nonaqueous, single-phase vehicle comprising at least one polymer and at least one solvent, the vehicle being miscible in water, wherein the drug is insoluble in one or more vehicle components and the conjugated C-peptide formulation is stable at 37° C. for at least two months. In one aspect, the solvent is selected from the group consisting of glycofurol, benzyl alcohol, tetraglycol, n-methylpyrrolidone, glycerol formal, propylene glycol, and combinations thereof.

In particular, a nonaqueous formulation is considered chemically stable if no more than about 35% of the conjugated C-peptide is degraded by chemical pathways, such as by oxidation, deamidation, and hydrolysis, after maintenance of the formulation at 37° C. for a period of two months, and a formulation is considered physically stable if, under the same conditions, no more than about 15% of the C-peptide contained in the formulation is degraded through aggregation. A drug formulation is stable according to the present invention if at least about 65% of the conjugated C-peptide remains physically and chemically stable after about two months at 37° C.

The conjugated C-peptide can be administered intranasally or by inhalation, typically in the form of a dry powder (either alone, as a mixture, e.g., in a dry blend with lactose, or as a mixed component particle, e.g., mixed with phospholipids, such as phosphatidylcholine) from a dry powder inhaler, as an aerosol spray from a pressurized container, pump, spray, atomizer (preferably an atomizer using electro hydrodynamics to produce a fine mist), or nebulizer, with or without the use of a suitable propellant, such as 1,1,1,2-tetrafluoroethane or 1,1,1,2,3,3,3-heptafluoropropane, or as nasal drops. For intranasal use, the powder may comprise a bioadhesive agent, e.g., chitosan or cyclodextrin. The pressurized container, pump, spray, atomizer, or nebulizer contains a solution or suspension of the compound(s) of the invention comprising, e.g., ethanol, aqueous ethanol, or a suitable alternative agent for dispersing, solubilizing, or extending release of the active, a propellant(s) as solvent and an optional surfactant, such as sorbitan trioleate, oleic acid, or an oligolactic acid.

Prior to use in a dry powder or suspension formulation, the drug product is micronized to a size suitable for delivery by inhalation (typically less than 5 μm). This may be achieved by any appropriate method, such as spiral jet milling, fluid bed jet milling, supercritical fluid processing to form nanoparticles, high pressure homogenization, or spray drying.

The particle size of conjugated C-peptide of this invention in the formulation delivered by the inhalation device is important with respect to the ability of C-peptide to make it into the lungs, and preferably into the lower airways or alveoli. Preferably, the conjugated C-peptide of this invention is formulated so that at least about 10% of the conjugated C-peptide delivered is deposited in the lung, preferably about 10% to about 20%, or more. It is known that the maximum efficiency of pulmonary deposition for mouth breathing humans is obtained with particle sizes of about 2 pm to about 3 pm. When particle sizes are above about 5 pm, pulmonary deposition decreases substantially. Particle sizes below about 1 pm cause pulmonary deposition to decrease, and it becomes difficult to deliver particles with sufficient mass to be therapeutically effective. Thus, particles of the conjugated C-peptide delivered by inhalation have a particle size preferably less than about 10 pm, more preferably in the range of about 1 pm to about 5 pm. The formulation of the conjugated C-peptide is selected to yield the desired particle size in the chosen inhalation device.

Advantageously for administration as a dry powder, a conjugated C-peptide of this invention is prepared in a particulate form with a particle size of less than about 10 pm, preferably about 1 to about 5 pm. The preferred particle size is effective for delivery to the alveoli of the patient's lung. Preferably, the dry powder is largely composed of particles produced so that a majority of the particles have a size in the desired range. Advantageously, at least about 50% of the dry powder is made of particles having a diameter less than about 10 pm. Such formulations can be achieved by spray drying, milling, or critical point condensation of a solution containing the conjugated C-peptide of this invention and other desired ingredients. Other methods also suitable for generating particles useful in the current invention are known in the art.

The particles are usually separated from a dry powder formulation in a container and then transported into the lung of a patient via a carrier air stream. Typically, in current dry powder inhalers, the force for breaking up the solid is provided solely by the patient's inhalation. In another type of inhaler, air flow generated by the patient's inhalation activates an impeller motor which deagglomerates the particles.

Capsules (made, e.g., from gelatin or hydroxypropylmethylcellulose), blisters and cartridges for use in an inhaler or insufflator may be formulated to contain a powder mix of the compound of the invention, a suitable powder base such as lactose or starch and a performance modifier such as 1-leucine, mannitol, or magnesium stearate. The lactose may be anhydrous or in the form of the monohydrate, preferably the latter. Other suitable excipients include dextran, glucose, maltose, sorbitol, xylitol, fructose, sucrose, and trehalose.

A suitable solution formulation for use in an atomizer using electro hydrodynamics to produce a fine mist may contain from 100 μg to 200 mg of conjugated C-peptide per actuation and the actuation volume may vary from 1 μL to 100 μL. A typical formulation may comprise conjugated C-peptide propylene glycol, sterile water, ethanol, and sodium chloride. Alternative solvents that may be used instead of propylene glycol include glycerol and polyethylene glycol. Suitable flavors, such as menthol and levomenthol, or sweeteners, such as saccharin or saccharin sodium, may be added to those formulations of the invention intended for inhaled/intranasal administration. Formulations for inhaled/intranasal administration may be formulated to be immediate and/or modified release using, e.g., PGLA. Modified release formulations include delayed, sustained, pulsed, controlled, targeted, and programmed release.

In the case of dry powder inhalers and aerosols, the dosage unit is determined by means of a valve that delivers a metered amount. Units in accordance with the invention are typically arranged to administer a metered dose or “puff” containing from 1 mg to 200 mg of conjugated C-peptide. The overall daily dose will typically be in the range 1 mg to 200 mg that may be administered in a single dose or, more usually, as divided doses throughout the day.

Examples of commercially available inhalation devices suitable for the practice of the invention are sold under the trademarks TURBHALER™ (Astra), ROTAHALER® (Glaxo), DISKUS®, SPIROS™ inhaler (Dura), devices marketed by Inhale Therapeutics under the trademarks AERX™ (Aradigm), and ULTRAVENT® nebulizer (Mallinckrodt), ACORN II® nebulizer (Marquest Medical Products), VENTOLIN® metered dose inhaler (Glaxo), and the SPINHALER® powder inhaler (Fisons), and the like.

Kits are also contemplated for this invention. A typical kit would comprise a container, preferably a vial, for the conjugated C-peptide formulation comprising conjugated C-peptide in a pharmaceutically acceptable formulation, and instructions, and/or a product insert or label. In one aspect, the instructions include a dosing regimen for administration of said conjugated C-peptide to an insulin-dependent patient to reduce the risk, incidence, or severity of hypoglycemia. In one aspect, the kit includes instructions to reduce the administration of insulin by about 5% to about 35% when starting conjugated C-peptide therapy. In another aspect, the instructions include directions for the patient to closely monitor their blood glucose levels when starting conjugated C-peptide therapy. In another aspect, the instructions include directions for the patient to avoid situations or circumstances that might predispose the patient to hypoglycemia when starting conjugated C-peptide therapy.

### EXAMPLES

Abbreviations. The following abbreviations have been used in the specification and examples: ACN=acetonitrile; Bzl=Bn=benzyl; DIEA=N,N-diisopropylethylamine; DMF=N,N-dimethylformamide; tBu=tert-butyl; TSTU=O—(N-succinimidyI)-1,1,3,3-tetramethyluronium tetrafluoroborate; THF=tetrahydrofuran; EtOAc=ethyl acetate; DIPEA=DIEA=diisopropylethylamine; HOAt=1-hydroxy-7-azabenzotriazole; NMP=N-methylpyrrolidin-2-one; TEA=triethyl amine; SA=sinapinic acid; Su=1-succinimidyl=2,5-dioxo-pyrrolidin-1-yl; TFA=trifluoracetic acid; DCM=dichioromethane; DMSO=dimethyl sulphoxide; RT=room temperature; General Procedures: The following examples and general procedures refer to intermediate compounds and final products identified in the specification. Alternatively, other reactions disclosed herein or otherwise conventional will be applicable to the preparation of the corresponding compounds of the invention. In all preparative methods, all starting materials are known or may easily be prepared from known starting materials. All temperatures are set forth in degrees Celsius (° C.) and unless otherwise indicated, all parts and percentages are by weight (i.e., w/w) when referring to yields and all parts are by volume (i.e., v/v) when referring to solvents and eluents.

### Example 1

### Preparation of PEGylated C-Peptides

Human C-peptide (SEQ. ID. No. 1) is dissolved in ACN/water (4:1). The pH was adjusted to 7.8 with N-methylmorpholine (NMM). A solution of N-hydroxysuccinimide activated ester PEG derivative in DMF/water/ACN (5:1:2) is added and the reaction is stirred overnight at room temperature.

The solution is diluted with purified water to a DMF concentration of 6% v/v. The pH is adjusted to 4 to 4.5 with acetic acid and filtered. The solution is purified by HPLC using a YMC-ODS column using an 0.5% acetic acid (mobile phase A)/100 mM sodium acetate (mobile phase B)/ACN (mobile phase C) gradient. Separations are completed by equilibrating the column with three column volumes of 90% A/10% C. PEGylated C-peptide is loaded on to, and washed with, 90% B/10% C (three column volumes), followed by isocratic washing with one column volume of 90% A/10% C. Elution is achieved via a multi-linear gradient starting with 90% A/10% C to 70% A/30% C, followed by a linear gradient consisting of 70% A/30% C and rising to 50% A/50% C over five column volumes.

The pool from the HPLC is diluted with purified water, evaporated, and lyophilized to yield PEGylated C-peptide.

Size exclusion chromatography is conducted using a Superdex 75, 10/300GL column using an isocratic elution with a mobile phase of 0.1 M phosphate buffer pH 7.4 containing 2.7 mM KCl and 0.137 M NaCl at a flow rate of 0.5 mL/min.

### Example 2

### Preparation of Human Serum Albumin Conjugates of C-Peptide

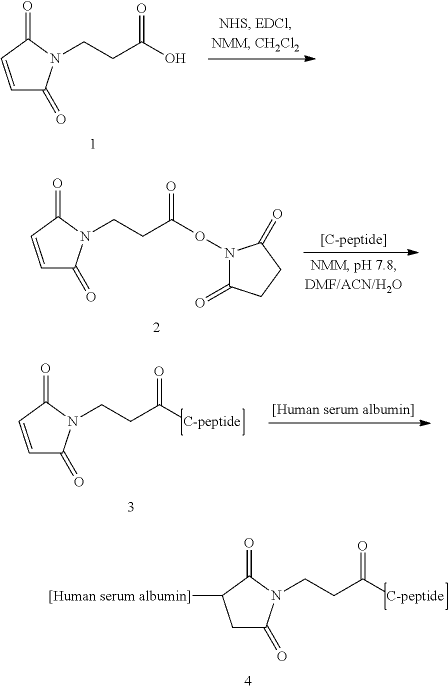

Compound 1 is treated with N-hydroxy succinimide (NHS), N-(3-dimethylamino-propyl-)-N′-ethylcarbodiimide (EDC), and N-methylmorpholine (NMM) in dichloromethane at room temperature to give compound 2. Compound 2 is reacted with C-peptide in DMF/water/ACN (5:1:2) which is adjusted to pH 7.8 with NMM, at room temperature, to give compound 3. Compound 3 is reacted with human serum albumin in aqueous solution at room temperature to give compound 4, which can be purified by any method known in the art.

### Example 3

### Preparation of Human Serum Albumin Conjugates of C-Peptide

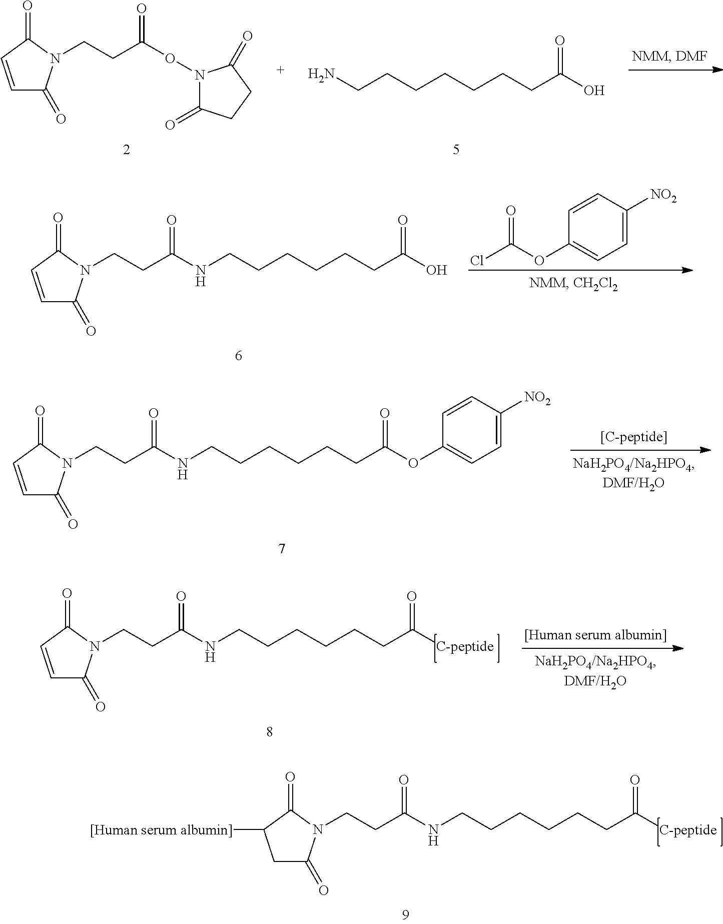

Compound 2 is reacted with compound 5 in the presence of NMM in DMF at room temperature to give compound 6. Compound 6 is reacted with para-nitrophenyl chloroformate in the presence of NMM in DMF at room temperature to give compound 7. Compound 7 is reacted with C-peptide in pH 7 phosphate buffered DMF/water, at room temperature, to give compound 8. Compound 8 is reacted with human serum albumin in pH 7 phosphate buffered DMF/water, at room temperature to give compound 9, which can be purified by any method known in the art.

### Example 4

### Preparation of Human Serum Albumin Conjugates of C-Peptide

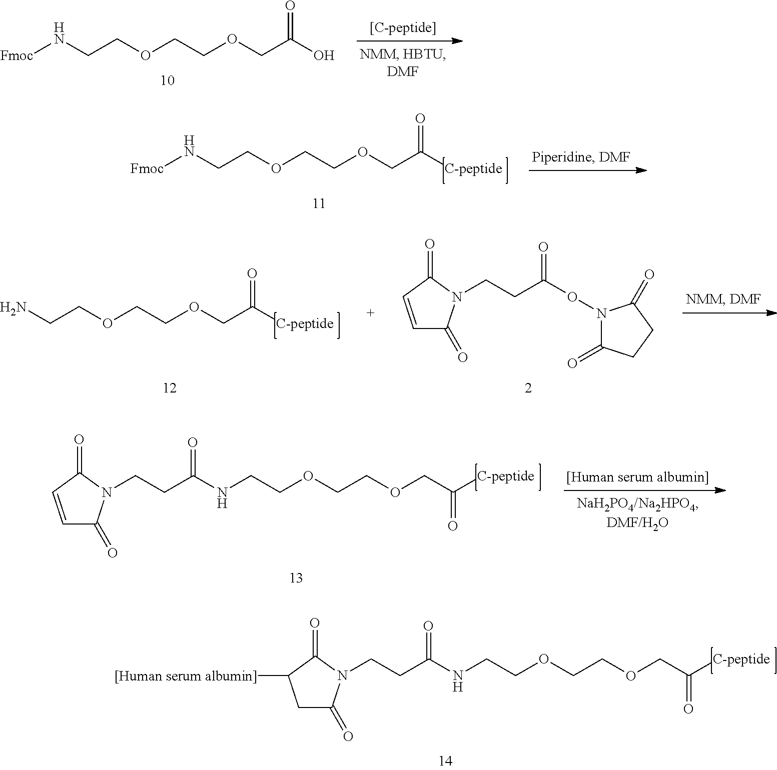

Compound 10 is treated with O-(benzotriazol-1-yl)-N,N,N′,N′-tetramethyluronium hexafluorophosphate, (H BTU) and N-methylmorpholine (NMM) in DMF, and then reacted with C-peptide at room temperature to give compound 11. Compound 11 is treated with 20% piperidine/DMF at room temperature to give compound 12. Compound 12 is reacted with compound 2 in the presence of NMM in DMF at room temperature to give compound 13. Compound 13 is reacted with human serum albumin in pH 7 phosphate buffered DMF/water, at room temperature to give compound 14, which can be purified by any method known in the art.

### Example 5

### Preparation of Human Serum Albumin Conjugates of C-Peptide

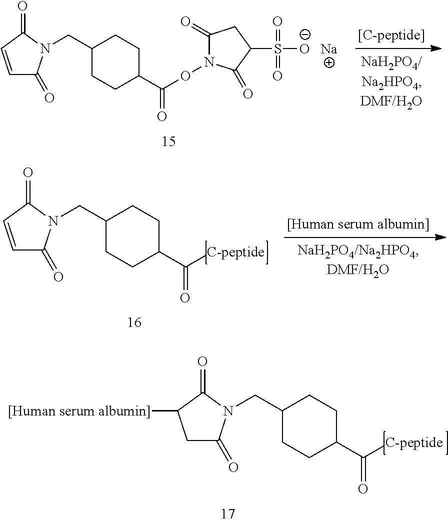

Compound 15 (sulfosuccinimidyl-4-(N-maleimidomethyl)cyclohexane-1-carboxylate (Sulfo-SMCC)) is reacted with C-peptide in pH 7 phosphate buffered DMF/water, at room temperature to give compound 16. Compound 16 is reacted with human serum albumin in pH 7 phosphate buffered DMF/water, at room temperature to give compound 17, which can be purified by any method known in the art.

### Example 6

### Preparation of Hydroxyethyl Starch Conjugates of C-Peptide

Hydroxyethyl starch (HES) conjugates of C-peptide can be prepared according to any of the methods described in US 20110200555 and Besheer et al., Bioconjugation Protocols: Strategies and Methods, Methods in Molecular Biology, vol. 751, chapter 2, 17-27, which are hereby incorporated by reference in their entireties.

### Example 7

### Preparation of Amino-Modified Hydroxyethyl Starch

Hydroxyethyl starch reagents functionalized with an amino group can be prepared as follows:
1. Dry 1 g of HES 70 (5.4 mM of the anhydroglucose units, AGU) at 105° C. for 2 h.
2. Dissolve the dried HES 70 in a solution containing 10 ml of dry DMF and 1 ml of triethylamine at 60° C.
3. Dissolve para-toluenesulfonyl chloride (0.5 g, 2.6 mM) in 1 ml of dry DMF.
4. Cool both solutions on ice to 0° C. and protect from light.
5. Add the para-toluenesulfonyl chloride solution gradually to the HES 70 solution and stir at 0° C. for 1 h.
6. Precipitate the polymer solution in 100 ml of cold acetone, filter, and wash with another 100 ml of acetone.
7. Dissolve the precipitate in water and dialyze against distilled water for 3 days (6-8 kDa MWCO), then lyophilize.
8. From the prepared HES tosylate, dissolve 200 mg in 30 ml DMF/borate buffer, pH 10 (1:2, v/v).
9. Add an excess of HMDA (hexamethylenediamine) (500 mg, 4.3 mM) dissolved in 10 ml of DMF/borate buffer, pH 10 (1:2, v/v), and stir overnight.
10. Precipitate the polymer in 200 ml of isopropanol/methanol (1:1, v/v), filter, and wash with 100 ml of the precipitating solvent.
11. Dry the precipitate at room temperature for 2 days.
12. Characterize the HES 70-amine product by 1H NMR (D20): d=1.27 (broad, 4H, NH(CH2)2-(CH2)2-(CH2)2-NH2), 1.5 (broad, 4H, NH—CH2-CH2-(CH2)2-CH2-CH2-NH2), 5.1-5.7 (broad, 1H, HC—anomeric carbon of AGU).

The prepared hydroxyethyl starch reagent can be further functionalized by peptide coupling with a bifunctional carboxylic acid (or monoprotected equivalent thereof) and further deprotection and/or coupling to C-peptide using conditions similar to those described thereof.

### Example 8

### Preparation of Oleic Acid Conjugates of C-Peptide

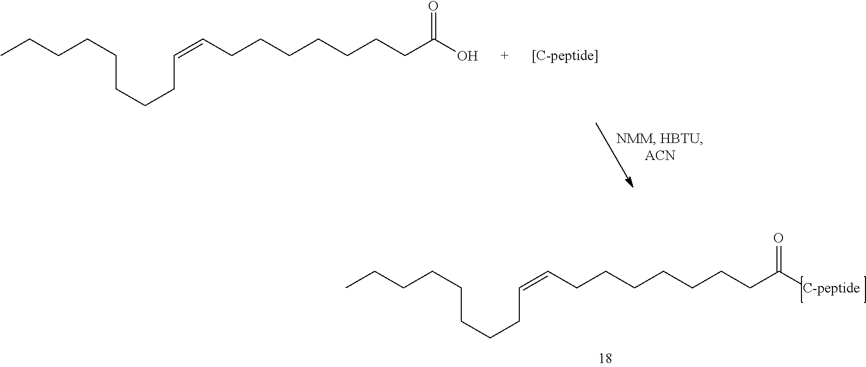

Oleic acid is treated with O-(benzotriazol-1-yl)-N,N,N′,N′-tetramethyluronium hexafluorophosphate, (HBTU) and N-methylmorpholine (NMM) in ACN, and then reacted with C-peptide at room temperature to give compound 11, which can be purified by any method known in the art.

### Example 9

### Measurement of Pharmacokinetic Characteristics in Dogs

A pharmacokinetic (PK) study was conducted to determine the C-peptide PK profile with unmodified C-peptide in beagle dogs.

Methods:

Two male and one female dog received S.C. the unmodified synthetic human C-peptide (0.5 mg/kg; 0.025 mL/kg) formulated in phosphate buffered saline (20 mg/mL). Dogs were bled by venipuncture and blood samples were collected at predetermined time points over 14 days. Plasma samples were obtained after centrifugation of the blood (3,000 rpm for 10 minutes) and stored at −80° C. until analysis. A CRO with Good Laboratory Practice (GLP) capabilities (MicroConstants, Inc.; San Diego, Calif.) performed the bioanalytical work. Plasma levels of C-peptide were measured by an enzyme-linked immunosorbant assay (ELISA) technique based on a commercial kit for human C-peptide determination (Mercodia; catalog number 10-1136-01) using the manufacturer's instructions. Results were expressed as C-peptide concentrations. For the PK analysis, the below quantitation level (BQL) was treated as zero and nominal time points were used for all calculations. PK parameters were determined by standard model independent methods based on the individual plasma concentration-time data for each animal using model 200 in WinNonlin Professional 5.2.1 (Pharsight Corp., Mountain View, Calif.).

Results:

All animals survived the duration of the study. Each treatment was well tolerated based on the absence of clinical abnormalities. The mean±standard deviation (SD) for C-peptide maximum concentration (Cₘₐₓ) and area under the curve (AUC₍₀₋ₜ₎) values following S.C. dosing of the unmodified C-peptide in dogs are presented in Table 3 below. Single-dose administration of unmodified C-peptide resulted in a rapid peak accumulation, and then rapid loss of C-peptide from the circulation in dogs. The use of unmodified C-peptide resulted in circulating levels of C-peptide that were BQL within half a day.

TABLE 3
Mean PK Parameters of C-Peptide in Dogs Following a Single
S.C. Dose of Unmodified Aqueous C-peptide (CP-AQ)

| | |
|---|---|
| Cₘₐₓ | AUC₍₀₋ₜ₎ |
| (ng/mL) | (ng · day/mL)^{a} |

| | Mean | SD | Mean | SD |
|---|---|---|---|---|
| CP-AQ | 757 | 192 | 77.4 | 6.82 |

^{a}AUC₍₀₋ₜ₎ is the area under the plasma concentration-time curve from immediate post dose to the last measurable sampling time and is calculated by the linear trapezoidal rule.

Additional PK studies conducted to determine the C-peptide PK profiles using representative conjugated C-peptides in beagle dogs using the techniques disclosed in PCT Application WO 2011146518, which is hereby incorporated by reference in its entirety. Results are then compared to those for unmodified C-peptide.

### Example 10

### Pharmacokinetics in Sprague Dawley Rats Following Single Dose s.c. Administration

The PK of the conjugated C-peptides can be assessed in Sprague Dawley rats using the techniques disclosed in PCT Application WO 2011146518, which is hereby incorporated by reference in its entirety.

### Example 11

### Pharmacokinetics in Cynomolgus Monkeys Following Single Dose s.c. Administration

The PK of the conjugated C-peptides can be assessed in Cynomolgus monkeys following single-dose s.c. administration using the techniques disclosed in PCT Application WO 2011146518, which is hereby incorporated by reference in its entirety.

### Example 12

### Repeat-Dose Pharmacokinetic Studies with Unmodified C-Peptide

GLP toxicology studies can be conducted with conjugated C-peptides in Cynomolgus monkeys using the techniques disclosed in PCT Application WO 2011146518, which is hereby incorporated by reference in its entirety.

### Example 13

### Repeat-Dose Pharmacokinetic Studies

The PK of the conjugated C-peptides can be assessed following multiple dose administration in rats and monkeys using the techniques disclosed in PCT Application WO 2011146518, which is hereby incorporated by reference in its entirety.

### Example 14

### Effect on Nerve Conduction Velocity (NCV) in STZ Induced Diabetic Rats

To assess the effect of the conjugated C-peptides on nerve conduction velocity in diabetic rats, the conjugated C-peptides are administered to STZ induced diabetic rats for 8 weeks using the techniques disclosed in PCT Application WO 2011146518, which is hereby incorporated by reference in its entirety. Results are also compared to those for unmodified human C-peptide. PEGylated rat C-peptide, which was coupled to the same 40 kDa branched PEG as described in Example 1, and unmodified rat C-peptide.

### Example 15

### Biophysical Characterization of Modified C-Peptide

Modified C-peptide prepared as described herein, is used in the analytical investigations described in Table 4.

TABLE 4
Structural testing

| Test | Analytical Technique |
|---|---|
| Molecular mass | MALDI-TOF MS |
| Identity | FT-IR |
| Identity and ratios of individual | Amino acid analysis for DS |
| amino acids | |
| Identity and chirality of individual | Chiral amino acid analysis |
| amino acids | |
| Molecular mass and sequence of amino | CID-MS/MS |
| acids (performed at the FI stage) | |
| Peptide Mapping (to confirm sequence | Chymotrysin digest followed by |
| on PEGylated peptide) | HPLC and MS/MS analysis of |
| | fragments |
| Absence of Counter ion | Ion chromatography, RP-HPLC, |
| | ICP-MS |

The structural tests listed above can be performed using the techniques disclosed in PCT Application WO 2011146518, which is hereby incorporated by reference in its entirety.

We claim:

1. A PEGylated C-peptide having a structure selected from the group consisting of structural Formula I and structural Formula II: 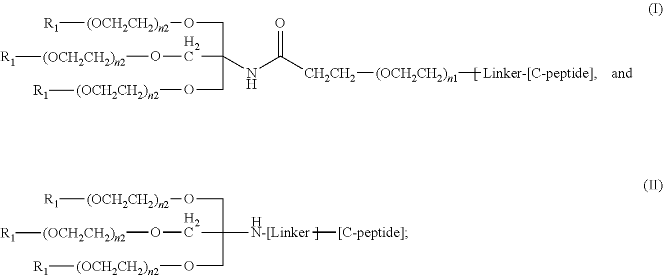
wherein:
R₁=alkyl;
n₁ is 1 to 12;
n₂ is 1 to 800;
the linker is selected from the group consisting of:
—X—, —CO—, —(CH₂)ₘ₂—,
—(CH₂)ₘ₁—CO—, —CO—(CH₂)ₘ₁—,
—CO—X—CO—, —(CH₂)ₘ₁—X—(CH₂)ₘ₁—,
—(CH₂)ₘ₁—CO—(CH₂)ₘ₁—, —X—CO—X—,
—X—(CH₂)ₘ₁—X—, —CO—(CH₂)ₘ₁—CO—,
—X—CO—(CH₂)ₘ₁—, —(CH₂)ₘ₁—CO—X—,
—X—(CH₂)ₘ₁—CO—X—, —X—CO—(CH₂)ₘ₁X—,
—X—CO—(CH₂)ₘ₁—CO—X—(CH₂)ₘ₁—X—CO—, —X—(CH₂)ₘ₁—X—CO—(CH₂)ₘ₂—,
—X—(CH₂)ₘ₁—CO—X—(CH₂)ₘ₂—, —X—(CH₂)ₘ₁—X—CO—(CH₂)ₘ₂—X—,
—X—(CH₂)ₘ₁—X—CO—(CH₂)ₘ₂—CO—,
—X—(CH₂)ₘ₁—CO—X—(CH₂)ₘ₂—X—, and
—X—(CH₂)ₘ₁—CO—X—(CH₂)ₘ₂—CO—;
wherein each X is independently selected from —O—, —S—, or NH— or is missing;
each m₁ is independently 0 to 5;
each m₂ is independently 1 to 5; and wherein the linker is attached to the N-terminal amino group of C-peptide.

2. A PEGylated C-peptide having structural Formula III: 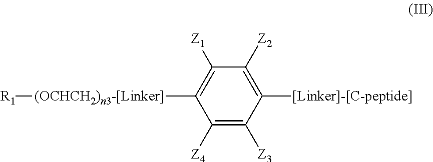
wherein:
R₁=alkyl;
n₁ is 1 to 12;
n₃ is 20 to 800;
Z₁, Z₂, Z₃, and Z₄ are independently selected from the group consisting of hydrogen and alkyl;
the linker is selected from the group consisting of:
—X—, —CO—, —(CH₂)ₘ₂—,
—(CH₂)ₘ₁—CO—, —CO—(CH₂)ₘ₁—,
—CO—X—CO—, —(CH₂)ₘ₁—X—(CH₂)ₘ₁—,
—(CH₂)ₘ₁—CO—(CH₂)ₘ₁—, —X—CO—X—,
—X—(CH₂)ₘ₁—X—, —CO—(CH₂)ₘ₁—CO—,
—X—CO—(CH₂)ₘ₁—, —(CH₂)ₘ₁—CO—X—,
—X—(CH₂)ₘ₁—CO—X—, —X—CO—(CH₂)ₘ₁X—,
—X—CO—(CH₂)ₘ₁—CO—X—(CH₂)ₘ₁—X—CO—, —X—(CH₂)ₘ₁—X—CO—(CH₂)ₘ₂—,
—X—(CH₂)ₘ₁—CO—X—(CH₂)ₘ₂—, —X—(CH₂)ₘ₁—X—CO—(CH₂)ₘ₂—X—,
—X—(CH₂)ₘ₁—X—CO—(CH₂)ₘ₂—CO—,
—X—(CH₂)ₘ₁—CO—X—(CH₂)ₘ₂—X—, and
—X—(CH₂)ₘ₁—CO—X—(CH₂)ₘ₂—CO—;
wherein each X is independently selected from —O—, —S—, or NH— or is missing;
each m₁ is independently 0 to 5;
each m₂ is independently 1 to 5; and wherein the linker is attached to the N-terminal amino group of C-peptide.

3. The PEGylated C-peptide of claim 2, wherein the PEGylated C-peptide has structural Formula IV:

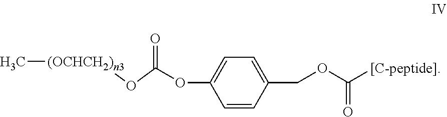

4. The PEGylated C-peptide of claim 2, wherein the PEGylated C-peptide has structural Formula V:

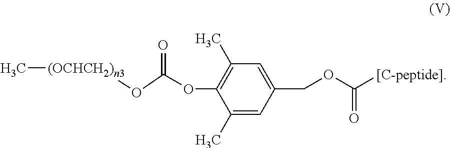

5. A PEGylated C-peptide having structural Formula VI:
CH₃(CH₂)₇CH═CH(CH₂)₈—O—(CH₂CH₂O)ₙ₁-[Linker]-[C-peptide] (VI)
wherein: n₁ is 20 to 800;
the linker is selected from the group consisting of:
—X—, —CO—, —(CH₂)ₘ₂—,
—CO—(CH₂)ₘ₁—,
—CO—X—CO—, —(CH₂)ₘ₁—X—(CH₂)ₘ₁—,
—(CH₂)ₘ₁—CO—(CH₂)ₘ₁—, —X—CO—X—,
—X—(CH₂)ₘ₁—X—, —CO—(CH₂)ₘ₁—CO—,
—X—CO—(CH₂)ₘ₁—, —(CH₂)ₘ₁—CO—X—,
—X—(CH₂)ₘ₁—CO—X—, —X—CO—(CH₂)ₘ₁X—,
—X—(CH₂)ₘ₁—X—CO—(CH₂)ₘ₂—,
—X—(CH₂)ₘ₁—CO—X—(CH₂)ₘ₂—,
—X—(CH₂)ₘ₁—X—CO—(CH₂)ₘ₂—CO—,
—X—(CH₂)ₘ₁—CO—X—(CH₂)ₘ₂—X—, and
—X—(CH₂)ₘ₁—CO—X—(CH₂)ₘ₂—CO—;
wherein each X is independently selected from —O—, —S—, or NH— or is missing;
each m₁ is independently 0 to 5;
each m₂ is independently 1 to 5; and wherein the linker is attached to the N-terminal amino group of C-peptide.

6. A PEGylated C-peptide having structural Formula VII: 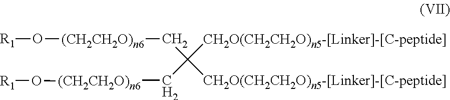
wherein:
R₁=alkyl;
n₅ is 1 to 12;
n₆ is 20 to 800;
the linker is selected from the group consisting of:
—X—, —CO—, —(CH₂)ₘ₂—,
—(CH₂)ₘ₁—CO—, —CO—(CH₂)ₘ₁—,
—CO—X—CO—, —(CH₂)ₘ₁—X—(CH₂)ₘ₁—,
—(CH₂)ₘ₁—CO—(CH₂)ₘ₁—, —X—CO—X—,
—X—(CH₂)ₘ₁—X—, —CO—(CH₂)ₘ₁—CO—,
—X—CO—(CH₂)ₘ₁—, —(CH₂)ₘ₁—CO—X—,
—X—(CH₂)ₘ₁—CO—X—, —X—CO—(CH₂)ₘ₁X—,
—X—CO—(CH₂)ₘ₁—CO—X—(CH₂)ₘ₁—X—CO—, —X—(CH₂)ₘ₁—X—CO—(CH₂)ₘ₂—,
—X—(CH₂)ₘ₁—CO—X—(CH₂)ₘ₂—, —X—(CH₂)ₘ₁—X—CO—(CH₂)ₘ₂—X—,
—X—(CH₂)ₘ₁—X—CO—(CH₂)ₘ₂—CO—,
—X—(CH₂)ₘ₁—CO—X—(CH₂)ₘ₂—X—, and
—X—(CH₂)ₘ₁—CO—X—(CH₂)ₘ₂—CO—;
wherein each X is independently selected from —O—, —S—, or NH— or is missing;
each m₁ is independently 0 to 5;
each m₂ is independently 1 to 5; and wherein the linker is attached to the N-terminal amino group of C-peptide.

7. A PEGylated C-peptide having structural Formula VIII: 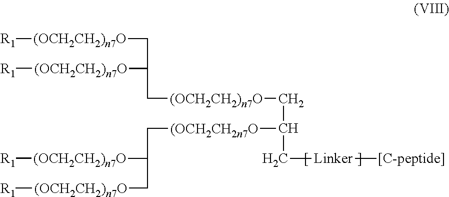
wherein:
R₁=alkyl;
n₇ is 1 to 800;
the linker is selected from the group consisting of:
—X—, —CO—, —(CH₂)ₘ₂—,
—(CH₂)ₘ₁—CO—, —CO—(CH₂)ₘ₁—,
—CO—X—CO—, —(CH₂)ₘ₁—X—(CH₂)ₘ₁—,
—(CH₂)ₘ₁—CO—(CH₂)ₘ₁—, —X—CO—X—,
—X—(CH₂)ₘ₁—X—, —CO—(CH₂)ₘ₁—CO—,
—X—CO—(CH₂)ₘ₁—, —(CH₂)ₘ₁—CO—X—,
—X—(CH₂)ₘ₁—CO—X—, —X—CO—(CH₂)ₘ₁X—,
—X—CO—(CH₂)ₘ₁—CO—X—(CH₂)ₘ₁—X—CO—, —X—(CH₂)ₘ₁—X—CO—(CH₂)ₘ₂—,
—X—(CH₂)ₘ₁—CO—X—(CH₂)ₘ₂—, —X—(CH₂)ₘ₁—X—CO—(CH₂)ₘ₂—X—,
—X—(CH₂)ₘ₁—X—CO—(CH₂)ₘ₂—CO—,
—X—(CH₂)ₘ₁—CO—X—(CH₂)ₘ₂—X—, and
—X—(CH₂)ₘ₁—CO—X—(CH₂)ₘ₂—CO—;
wherein each X is independently selected from —O—, —S—, or NH— or is missing;
each m₁ is independently 0 to 5;
each m₂ is independently 1 to 5; and wherein the linker is attached to the N-terminal amino group of C-peptide.

8. A PEGylated C-peptide having structural Formula IX: 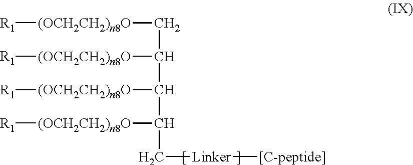
wherein:
R₁=alkyl;
n₇ is 1 to 800;
the linker is selected from the group consisting of:
—X—, —CO—, —(CH₂)ₘ₂—,
—(CH₂)ₘ₁—CO—, —CO—(CH₂)ₘ₁—,
—CO—X—CO—, —(CH₂)ₘ₁—X—(CH₂)ₘ₁—,
—(CH₂)ₘ₁—CO—(CH₂)ₘ₁—, —X—CO—X—,
—X—(CH₂)ₘ₁—X—, —CO—(CH₂)ₘ₁—CO—,
—X—CO—(CH₂)ₘ₁—, —(CH₂)ₘ₁—CO—X—,
—X—(CH₂)ₘ₁—CO—X—, —X—CO—(CH₂)ₘ₁X—,
—X—CO—(CH₂)ₘ₁—CO—X—(CH₂)ₘ₁—X—CO—, —X—(CH₂)ₘ₁—X—CO—(CH₂)ₘ₂—,
—X—(CH₂)ₘ₁—CO—X—(CH₂)ₘ₂—, —X—(CH₂)ₘ₁—X—CO—(CH₂)ₘ₂—X—,
—X—(CH₂)ₘ₁—X—CO—(CH₂)ₘ₂—CO—,
—X—(CH₂)ₘ₁—CO—X—(CH₂)ₘ₂—X—, and
—X—(CH₂)ₘ₁—CO—X—(CH₂)ₘ₂—CO—;
wherein each X is independently selected from —O—, —S—, or NH— or is missing;
each m₁ is independently 0 to 5;
each m₂ is independently 1 to 5; and wherein the linker is attached to the N-terminal amino group of C-peptide.

9. A PEGylated C-peptide having a structure selected from the group consisting of structural Formula X and structural Formula XI: 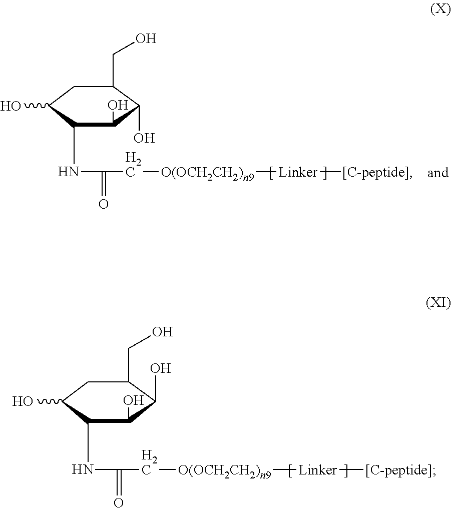
wherein: n₉ is 20 to 800;
the linker is selected from the group consisting of:
—X—, —CO—, —(CH₂)ₘ₂—,
—(CH₂)ₘ₁—CO—, —CO—(CH₂)ₘ₁—,
—CO—X—CO—, —(CH₂)ₘ₁—X—(CH₂)ₘ₁—,
—(CH₂)ₘ₁—CO—(CH₂)ₘ₁—, —X—CO—X—,
—X—(CH₂)ₘ₁—X—, —CO—(CH₂)ₘ₁—CO—,
—X—CO—(CH₂)ₘ₁—, —(CHOₘ₁—CO—X—,
—X—(CHOₘ₁—CO—X—, —X—CO—(CH₂)ₘ₁X—,
—X—CO—(CH₂)ₘ₁—CO—X—(CH₂)ₘ₁—X—CO—, —X—(CH₂)ₘ₁—X—CO—(CH₂)ₘ₂—,
—X—(CH₂)ₘ₁—CO—X—(CH₂)ₘ₂—, —X—(CH₂)ₘ₁—X—CO—(CH₂)ₘ₂—X—,
—X—(CH₂)ₘ₁—X—CO—(CH₂)ₘ₂—CO—,
—X—(CH₂)ₘ₁—CO—X—(CH₂)ₘ₂—X—, and
—X—(CH₂)ₘ₁—CO—X—(CH₂)ₘ₂—CO—;
wherein each X is independently selected from —O—, —S—, or NH— or is missing;
each m₁ is independently 0 to 5;
each m₂ is independently 1 to 5; and wherein the linker is attached to the N-terminal amino group of C-peptide.

10. A PEGylated C-peptide having structural Formula XII: 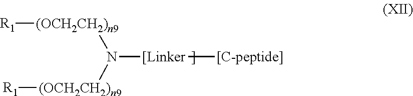
wherein:
R₁=alkyl;
n₉ is 20 to 800;
the linker is selected from the group consisting of:
—X—, —CO—, —(CH₂)ₘ₂—,
—(CH₂)ₘ₁—CO—, —CO—(CH₂)ₘ₁—,
—CO—X—CO—, —(CH₂)ₘ₁—X—(CH₂)ₘ₁—,
—(CH₂)ₘ₁—CO—(CH₂)ₘ₁—, —X—CO—X—,
—X—(CH₂)ₘ₁—X—, —CO—(CH₂)ₘ₁—CO—,
—X—CO—(CH₂)ₘ₁—, —(CH₂)ₘ₁—CO—X—,
—X—(CH₂)ₘ₁—CO—X—, —X—CO—(CH₂)ₘ₁X—,
—X—CO—(CH₂)ₘ₁—CO—X—(CH₂)ₘ₁—X—CO—, —X—(CH₂)ₘ₁—X—CO—(CH₂)ₘ₂—,
—X—(CH₂)ₘ₁—CO—X—(CH₂)ₘ₂—, —X—(CH₂)ₘ₁—X—CO—(CH₂)ₘ₂—X—,
—X—(CH₂)ₘ₁—X—CO—(CH₂)ₘ₂—CO—,
—X—(CH₂)ₘ₁—CO—X—(CH₂)ₘ₂—X—, and
—X—(CH₂)ₘ₁—CO—X—(CH₂)ₘ₂—CO—;
wherein each X is independently selected from —O—, —S—, or NH— or is missing;
each m₁ is independently 0 to 5;
each m₂ is independently 1 to 5; and wherein the linker is attached to the N-terminal amino group of C-peptide.

11. A modified C-peptide having the structural Formula XIII: 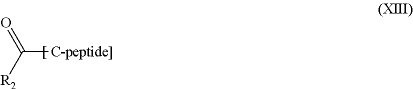
Wherein:
The C-peptide is modified at the N-terminus; and
R₂ is selected from the group consisting of alkyl, haloalkyl, perhaloalkyl, heteroalkyl, hydroxyalkyl, alkoxyalkyl, aminoalkyl, alkylaminoalkyl, thioalkyl, arylalkyl, cycloalkylalkyl, heteroarylalkyl, heterocycloalkylalkyl, alkenyl, arylalkenyl, heteroarylalkenyl, heterocycloalkylalkenyl, alkynyl, arylalkynyl, heteroarylalkynyl, heterocycloalkylalkynyl, alkoxy, haloalkoxy, perhaloalkoxy, arylalkoxy, aryloxy, heteroaryloxy, alkylamino, alkylthio, arylthio, heteroarylthio, aryl, heteroaryl, cycloalkyl, and heterocycloalkyl, any of which may be optionally substituted with a substituent selected from the group consisting of hydrogen, halogen, hydroxy, cyano, nitro, alkyl, haloalkyl, perhaloalkyl, heteroalkyl, hydroxyalkyl, alkoxyalkyl, aminoalkyl, alkylaminoalkyl, thioalkyl, arylalkyl, cycloalkylalkyl, heteroarylalkyl, heterocycloalkylalkyl, alkenyl, arylalkenyl, heteroarylalkenyl, heterocycloalkylalkenyl, alkynyl, arylalkynyl, heteroarylalkynyl, heterocycloalkylalkynyl, alkoxy, haloalkoxy, perhaloalkoxy, acyloxy, arylalkoxy, aryloxy, heteroaryloxy, acyl, arylalkanoyl, alkylcarbonyl, alkoxycarbonyl, carboxyl, amino, alkylamino, arylamino, C-amido, N-amido, carbamate, urea, N-sulfonamido, S-sulfonamido, alkylsulfonyl, thiol, alkylthio, arylthio, heteroarylthio, aryl, heteroaryl, cycloalkyl, and heterocycloalkyl.

12. The modified C-peptide of claim 11, wherein R₂ is alkyl.

13. The modified C-peptide of claim 12, wherein R₂ is unsubstituted C₈-C₂₀ alkyl.

14. The modified C-peptide of claim 13, wherein R₂ is selected from the group consisting of cis-CH₃(CH₂)₃CH═CH(CH₂)₇—, cis-CH₃(CH₂)₅CH═CH(CH₂)₇—, cis-CH₃(CH₂)₇CH═CH(CH₂)₇—, cis,cis-CH₃(CH₂)₄CH═CHCH₂CH═CH(CH₂)₇, CH₃(CH₂)₆—, CH₃(CH₂)₈—, CH₃(CH₂)₁₀—, CH₃(CH₂)₁₂—, CH₃(CH₂)₁₄—, CH₃(CH₂)₁₆—, CH₃(CH₂)₁₈—, and CH₃(CH₂)₂₀—.

15. The modified C-peptide of claim 13, wherein R₂ is selected from the group consisting of CH₃(CH₂)₁₆—.

16. The modified C-peptide of claim 13, having the structure:

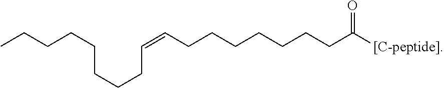

17. A modified C-peptide having the structural Formula XIV:
[Human Serum Albumin]-[Linker]-[C-peptide] (XIV)
wherein:
the linker is selected from the group consisting of:
-Q₁-(CH₂)ₘ₁—CO—,
—CO—X—CO—, -Q₁-(CH₂)ₘ₁—X—(CH₂)ₘ₁—,
-Q₁-(CH₂)ₘ₁—CO—(CH₂)ₘ₁—, -Q₁-X—CO—X—,
-Q₁-X—(CH₂)ₘ₁—X—, —CO—(CH₂)ₘ₁—CO—,
-Q₁-X—CO—(CH₂)ₘ₁—, -Q₁-(CH₂)ₘ₁—CO—X—,
-Q₁-X—(CH₂)ₘ₁—CO—X—, -Q₁-X—CO—(CH₂)ₘ₁X—,
-Q₁-X—CO—(CH₂)ₘ₁—CO—X—(CH₂)ₘ₁—X—CO—, -Q₁-X—(CH₂)ₘ₁—X—CO—(CH₂)ₘ₂—,
-Q₁-X—(CH₂)ₘ₁—CO—X—(CH₂)ₘ₂—, -Q₁-X—(CH₂)ₘ₁—X—CO—(CH₂)ₘ₂—X—,
-Q₁-X—(CH₂)ₘ₁—X—CO—(CH₂)ₘ₂—CO—,
-Q₁-X—(CH₂)ₘ₁—CO—X—(CH₂)ₘ₂—X—, and
-Q₁-X—(CH₂)ₘ₁—CO—X—(CH₂)ₘ₂—CO—;
wherein Q₁ is absent or selected from the group consisting of —CO— or 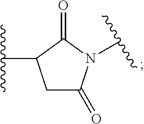 [SMILES: *C1CC(=O)N(*)C1=O]
each X is independently selected from —O—, —S—, or NH— or is missing;
each m₁ is independently 0 to 5;
each m₂ is independently 1 to 5; and
wherein the linker is attached to the N-terminal amino group of C-peptide.

18. The modified C-peptide of claim 17, wherein the human serum albumin is modified at the 34-cysteine.

19. The modified C-peptide of claim 17, having the structure:

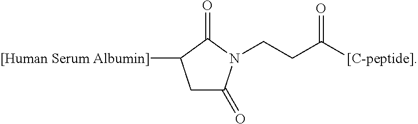

20. The modified C-peptide of claim 17, having the structure:

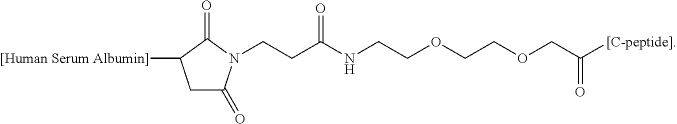

21. The modified C-peptide of claim 17, having the structure:

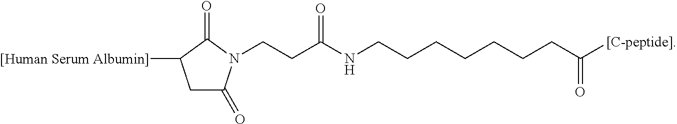

22. The modified C-peptide of claim 17, having the structure:

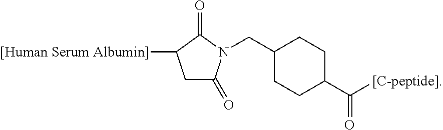

23. A modified C-peptide having the structural Formula XIII:
[Hydroxyethyl Starch]-[Linker]-[C-peptide] (XV)
the linker is selected from the group consisting of:
—X—, —CO—, —(CH₂)ₘ₂—,
—(CH₂)ₘ₁—CO—, —CO—(CH₂)ₘ₁—,
—CO—X—CO—, —(CH₂)ₘ₁—X—(CH₂)ₘ₁—,
—(CH₂)ₘ₁—CO—(CH₂)ₘ₁—, —X—CO—X—,
—X—(CH₂)ₘ₁—X—, —CO—(CH₂)ₘ₁—CO—,
—X—CO—(CH₂)ₘ₁—, —(CH₂)ₘ₁—CO—X—,
—X—(CH₂)ₘ₁—CO—X—, —X—CO—(CH₂)ₘ₁ X—,
—X—CO—(CH₂)ₘ₁—CO—X—(CH₂)ₘ₁—X—CO—, —X—(CH₂)ₘ₁—X—CO—(CH₂)ₘ₂—,
—X—(CH₂)ₘ₁—CO—X—(CH₂)ₘ₂—, —X—(CH₂)ₘ₁—X—CO—(CH₂)ₘ₂—X—,
—X—(CH₂)ₘ₁—X—CO—(CH₂)ₘ₂—CO—,
—X—(CH₂)ₘ₁—CO—X—(CH₂)ₘ₂—X—, and
—X—(CH₂)ₘ₁—CO—X—(CH₂)ₘ₂—CO—;
wherein each X is independently selected from —O—NH—, —NH—NH—, —O—, —S—, or NH— or is missing;
each m₁ is independently 0 to 5;
each m₂ is independently 1 to 5; and wherein the linker is attached to the N-terminal amino group of C-peptide.

24. The modified C-peptide of claim 23, wherein said hydroxyethyl starch has a molecular weight of about 70 kDa to 200 kDa.

25. The modified C-peptide of claim 24, wherein said hydroxyethyl starch has a molecular weight of about 70 kDa.

26. The modified C-peptide of claim 24, wherein said hydroxyethyl starch has a molecular weight of about 130 kDa.

27. The modified C-peptide of claim 24, wherein said hydroxyethyl starch has a molecular weight of about 200 kDa.

28. A composition comprising hydroxyethyl starch and the modified C-peptide of any of claims 23 to 27, wherein the modified C-peptide comprises a molar percentage of about 0.3% to about 0.5%.

29. The modified C-peptide of any of claims 1 to 28, wherein the C-peptide comprises the pentapeptide sequence (EGSLQ) (SEQ. ID. No. 31).

30. The modified C-peptide of any of claims 1 to 29, wherein the modified C-peptide has substantially the same secondary structure as unmodified C-peptide, as determined via UV circular dichroism analysis.

31. The modified C-peptide of any of claims 1 to 29, wherein the modified C-peptide has a plasma or sera pharmacokinetic AUC profile at least 10-fold greater than unmodified C-peptide when subcutaneously administered to dogs.

32. The modified C-peptide of any of claims 1 to 29, wherein the modified C-peptide retains at least about 50% of the biological activity of the unmodified C-peptide.

33. The modified C-peptide of any of claims 1 to 29, wherein the modified C-peptide retains at least about 75% of the biological activity of the unmodified C-peptide.

34. A dosing regimen which maintains an average steady-state concentration of modified C-peptide in the patient's plasma of between about 0.2 nM and about 6 nM when using a dosing interval of 3 days or longer, comprising administering to the patient a therapeutic dose of modified C-peptide of any of claims 1 to 29.

35. A method for maintaining C-peptide levels above the minimum effective therapeutic level in a patient in need thereof, comprising administering to the patient a therapeutic dose of modified C-peptide of any of claims 1 to 29.

36. A method for treating one or more long-term complications of diabetes in a patient in need thereof, comprising administering to the patient a therapeutic dose of modified C-peptide of any of claims 1 to 29.

37. The method of claim 36, wherein the long-term complications of diabetes are selected from the group consisting of retinopathy, peripheral neuropathy, autonomic neuropathy, and nephropathy.

38. The method of claim 37, wherein the long-term complications of diabetes is peripheral neuropathy.

39. The method of claim 37, wherein the peripheral neuropathy is established peripheral neuropathy.

40. The method of claim 39, wherein treatment results in an improvement of at least 10% in nerve conduction velocity compared to nerve conduction velocity prior to starting modified C-peptide therapy.

41. A method for treating a patient with diabetes comprising administering to the patient a therapeutic dose of modified C-peptide of any of claims 1 to 29 in combination with insulin.

42. A method for treating an insulin-dependent human patient, comprising the steps of;
a) administering insulin to the patient, wherein the patient has neuropathy;
b) administering subcutaneously to the patient a therapeutic dose of modified C-peptide of any of claims 1 to 29 in a different site as that used for the patient's insulin administration;
c) adjusting the dosage amount, type, or frequency of insulin administered based on monitoring the patient's altered insulin requirements resulting from the therapeutic dose of modified C-peptide, wherein the adjusted dose of insulin reduces the risk, incidence, or severity of hypoglycemia, wherein the adjusted dose of insulin is at least 10% less than the patient's insulin dose prior to starting modified C-peptide treatment.

43. The method of claim 41 or 42, wherein the insulin is administered subcutaneously at a different depot site compared to that most recently used for the modified C-peptide.

44. The method of any of claims 36 to 43, wherein the modified C-peptide is administered with a dosing interval of about 3 days or longer.

45. The method of any of claims 36 to 43, wherein the modified C-peptide is administered with a dosing interval of about 5 days or longer.

46. The method of any of claims 36 to 43, wherein the modified C-peptide is administered with a dosing interval of about 7 days or longer.

47. The method of any of claims 36 to 46, wherein the therapeutic dose of modified C-peptide is administered subcutaneously.

48. The modified C-peptide of any of claims 1 to 29, for use as a C-peptide replacement therapy in a patient in need thereof.

49. Use of the modified C-peptide of any of claims 1 to 29 to reduce the risk of hypoglycemia in a human patient with insulin dependent diabetes, in a regimen which additionally comprises the administration of insulin, comprising;
a) administering insulin to the patient;
b) administering a therapeutic dose of the modified C-peptide in a different site as that used for the patient's insulin administration;
c) adjusting the dosage amount, type, or frequency of insulin administered based on the patient's altered insulin requirements resulting from the therapeutic dose of the modified C-peptide.

50. The use of claim 49, wherein the patient has at least one long term complications of diabetes.

51. Use of the modified C-peptide of any of claims 1 to 29 for treating one or more long-term complications of diabetes in a patient in need thereof.

52. The use of any of claims 49 to 51, wherein the long-term complications of diabetes are selected from the group consisting of retinopathy, peripheral neuropathy, autonomic neuropathy, nephropathy and erectile dysfunction.

53. The use of claim 52, wherein the long-term complications of diabetes is peripheral neuropathy.

54. The use of claim 52, wherein the peripheral neuropathy is established peripheral neuropathy.

55. The use of claim 54, wherein treatment results in an improvement of at least 10% in nerve conduction velocity compared to nerve conduction velocity prior to starting modified C-peptide therapy

56. A pharmaceutical composition comprising the modified C-peptide of any of claims 1 to 29 and a pharmaceutically acceptable carrier or excipient.

57. A pharmaceutical composition comprising the modified C-peptide of any of claims 1 to 29 and insulin.

58. A method of reducing insulin usage in an insulin-dependent human patient, comprising the steps of;
a) administering insulin to the patient;
b) administering subcutaneously to the patient a therapeutic dose of the modified C-peptide of any of claims 1 to 29 in a different site as that used for the patient's insulin administration;
c) adjusting the dosage amount, type, or frequency of insulin administered based on monitoring the patient's altered insulin requirements resulting from the therapeutic dose of modified C-peptide, wherein the adjusted dose of insulin does not induce hypoglycemia, wherein the adjusted dose of insulin is at least 10% less than the patient's insulin dose prior to starting the modified C-peptide treatment.


SEQUENCE LISTING (33 sequences)

SEQ ID NO: 1 - protein, 31 aa, Homo sapiens
EAEDLQVGQVELGGGPGAGSLQPLALEGSLQ

SEQ ID NO: 2 - protein, 31 aa, Pan troglodytes; identical to SEQ ID NO: 1

SEQ ID NO: 3 - protein, 31 aa, Gorilla gorilla; identical to SEQ ID NO: 1

SEQ ID NO: 4 - protein, 31 aa, Pongo pygmaeus; identical to SEQ ID NO: 1

SEQ ID NO: 5 - protein, 31 aa, Chlorocebus aethiops
EAEDPQVGQVELGGGPGAGSLQPLALEGSLQ

SEQ ID NO: 6 - protein, 31 aa, Canis lupus familiaris
EVEDLQVRDVELAGAPGEGGLQPLALEGALQ

SEQ ID NO: 7 - protein, 31 aa, Oryctolagus cuniculus
EVEELQVGQAELGGGPDAGGLQPSALELALQ

SEQ ID NO: 8 - protein, 31 aa, Rattus norvegicus
EVEDPQVAQLELGGGPGAGDLQTLALEVARQ

SEQ ID NO: 9 - protein, 31 aa, Apodemus semotus; identical to SEQ ID NO: 8

SEQ ID NO: 10 - protein, 31 aa, Geodia cydonium
EVEDPQVGQVELGAGPGAGSEQTLALEVARQ

SEQ ID NO: 11 - protein, 27 aa, Mus musculus
EVEDPQVAQLELGGGPGAGDLQTLALE

SEQ ID NO: 12 - protein, 27 aa, Mus caroli
EVEDPQVAQLELGGGPGAGDLQTLALE

SEQ ID NO: 13 - protein, 31 aa, Rattus norvegicus
EVEDPQVPQLELGGGPGAGDLQTLALEVARQ

SEQ ID NO: 14 - protein, 31 aa, Rattus losea
EVEDPQVAQQELGGGPGAGDLQTLALEVARQ

SEQ ID NO: 15 - protein, 31 aa, Niviventer coxingi
EVEDPQVPQLELGGGPGTGDLQTLALEVARQ

SEQ ID NO: 16 - protein, 26 aa, Microtus kikuchii
VEDPQVAQLELGGGPGAGDLQTLALE

SEQ ID NO: 17 - protein, 31 aa, Rattus norvegicus
EVEDPQVPQLELGGGPEAGDLQTLALEVARQ

SEQ ID NO: 18 - protein, 31 aa, Felis catus
EAEDLQGKDAELGEAPGAGGLQPSALEAPLQ

SEQ ID NO: 19 - protein, 26 aa, Mesocricetus auratus
VEDPQVAQLELGGGPGADDLQTLALE

SEQ ID NO: 20 - protein, 31 aa, Niviventer coxingi
EVEDPQVAQLELGEGPEAGDLQTLALEVARQ

SEQ ID NO: 21 - protein, 31 aa, Apodemus semotus
EVEDPQVEQLELGGAPGTGDLETLALEVARQ

SEQ ID NO: 22 - protein, 31 aa, Rattus losea
EVEDPQVPQLELGGSPEAGDLQTLALEVARQ

SEQ ID NO: 23 - protein, 30 aa, Meriones unguiculatus
VEDPQMPQLELGGSPGAGDLQALALEVARQ

SEQ ID NO: 24 - protein, 30 aa, Psammomys obesus
VDDPQMPQLELGGSPGAGDLRALALEVARQ

SEQ ID NO: 25 - protein, 26 aa, Sus scrofa
EAENPQAGAVELGGGLGGLQALALEG

SEQ ID NO: 26 - protein, 31 aa, Rhinolophus ferrumequinum
EVEDPQAGQVELGGGPGTGGLQSLALEGPPQ

SEQ ID NO: 27 - protein, 31 aa, Equus przewalskii
EAEDPQVGEVELGGGPGLGGLQPLALAGPQQ

SEQ ID NO: 28 - protein, 26 aa, Bos taurus
EVEGPQVGALELAGGPGAGGLEGPPQ

SEQ ID NO: 29 - protein, 31 aa, Otolemur garnettii
DTEDPQVGQVGLGGSPITGDLQSLALDVPPQ

SEQ ID NO: 30 - protein, 31 aa, Artificial Sequence
  Synthesized
EXEXXQXXXXELXXXXXXXXXXXXALBXXXQ

SEQ ID NO: 31 - protein, 5 aa, Artificial Sequence
  Synthesized
EGSLQ

SEQ ID NO: 32 - protein, 12 aa, Artificial Sequence
  Synthesized
EAEDLQVGQVEL

SEQ ID NO: 33 - protein, 31 aa, Artificial Sequence
  Synthesized
GXEXXQXXXXELXXXXXXXXXXXXALBXXXQ